(19) United States
(12) Patent Application Publication
(10) Pub. No.: US 2025/0295657 A1
(43) Pub. Date: Sep. 25, 2025

(54) Methods and Compositions for Treatment of Inflammation and Inflammatory Conditions

(71) Applicant: University of Pittsburgh - Of the Commonwealth System of Higher Education, Pittsburgh, PA (US)

(72) Inventors: Stephen Yu-Wah Chan, Pittsburgh, PA (US); Ivet Bahar, Setauket, NY (US); Lloyd David Harvey, Pittsburgh, PA (US)

(21) Appl. No.: 18/869,613

(22) PCT Filed: May 26, 2023
(86) PCT No.: PCT/IB2023/055431

§ 371 Date: Nov. 26, 2024

Related U.S. Application Data
(60) Provisional application No. 63/346,453, filed on May 27, 2022.
(60) Provisional application No. 63/502,499, filed on May 16, 2023.

(51) Int. Cl. A61K 31/502; A61P 29/00; C07D 405/12

(52) U.S. Cl. CPC A61K 31/502; A61P 29/00; C07D 405/12

(57) ABSTRACT

Provided herein are methods of treating a patient, such as a human patient, having inflammation or an inflammatory disease or a disease in which inflammation is present, such as cardiovascular or vascular endothelium inflammation, such as pulmonary hypertension, restenosis, essential hypertension, atherosclerosis, stroke, sepsis, or a viral infection, such as a coronavirus infection, SARS-CoV-2. The methods comprise administering to the patient an amount of a compound as described herein effective to treat the patient. NCOA7-activating compounds and compositions also are provided. Also provided is a gene editing method of reducing inflammation or an inflammatory condition in a patient having a C at SNP rs 11154337, comprising editing the C to a G.

### CROSS REFERENCE TO RELATED APPLICATIONS

This application is the United States national phase of International Patent Application No. PCT/IB2023/055431, filed May 26, 2023, and claims priority to U.S. Provisional Patent Application No. 63/346,453 filed May 27, 2022, and U.S. Provisional Patent Application No. 63/502,499 filed May 16, 2023, the disclosures of which are hereby incorporated herein by reference in their entireties.

### STATEMENT REGARDING FEDERAL FUNDING

This invention was made with government support under Grant No. HL138437, awarded by the National Institutes of Health. The government has certain rights in the invention.

The Sequence Listing associated with this application is filed in electronic format via Patent Center and is hereby incorporated by reference into the specification in its entirety. The name of the XML file containing the Sequence Listing is 2302529.xml. The size of the XML file is 31,855 bytes and the XML file was created on May 25, 2023.

Inflammation is a component of a significant part of a vast number of diseases including vascular disease and cardiac disease, but also including non-vascular diseases like sepsis, COVID (coronavirus disease), ARDS (acute respiratory distress syndrome), acute lung injury, stroke, neurodegeneration, cancer, and autoimmune diseases. In the example of vascular inflammation, it regulates key vascular and endothelial pathophenotypes (such as in atherosclerosis, essential hypertension, peripheral vascular disease, and restenosis, among others), but the precise causative mechanisms remain enigmatic particularly in pulmonary vascular diseases such as pulmonary arterial hypertension (PAH), or other forms (groups 2-5) of pulmonary hypertension. In one example, PAH includes as a class: idiopathic PAH, heritable PAH (e.g., BMPR-2, etc.), diseases such as connective tissue disorder-associated PAH, HIV infection, portopulmonary hypertension, congenital heart disease, schistosomiasis, and hemolytic anemia, pulmonary veno-occlusive disease, and persistent pulmonary hypertension of the newborn.

Identification of active pharmaceutical ingredients, e.g. drugs, capable of reducing inflammation, e.g., vascular or vascular endothelial inflammation would provide useful therapeutics for treatment of a variety of diseases, such as in treating inflammation and inflammatory diseases, cardiovascular inflammation, vascular inflammation (e.g., having vascular endothelium inflammation or a disease having vascular endothelium inflammation as a symptom, such as pulmonary hypertension (PH, including Groups 1-5 types of PH, e.g., pulmonary arterial hypertension (PAH), pulmonary hypertension due to left heart disease, pulmonary hypertension due to lung disease, pulmonary hypertension due to chronic blood clots in the lungs, and pulmonary hypertension due to unknown causes), restenosis, essential hypertension, atherosclerosis, and stroke), a disease characterized by vascular inflammation, or a disease of innate and acquired immunity, such as autoimmune diseases, heart disease, lung disease, sepsis, cancer, and neurodegeneration, e.g., inflammation associated with, for example, heart disease, lung disease, sepsis, cancer, and neurodegeneration. Identification of such useful active pharmaceutical ingredients, and related methods of use are desired.

### SUMMARY

A method of treating inflammation in a patient, such as a human patient, is provided. The method comprising:
administering to the patient an amount of a compound, having the structure:

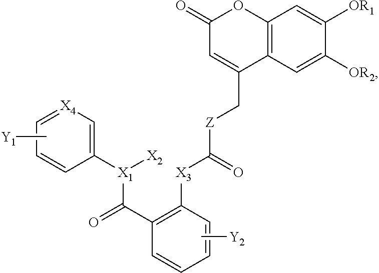

wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl, —NHR₆ where R₆ is —H or —C₁₋₃ alkyl, —NHC(O)—R₇ where R₇ is —H or —C₁₋₃ alkyl, —OR₈ where Rₐ is —H or —C₁₋₃ alkyl, —OC(O)—R₉ where R₉ is —H or —C₁₋₃ alkyl, —C(O)—R₁₀ where R₁₀ is —H or —C₁₋₃ alkyl, or —C(O)—R₁₁—R₁₂ where R₁₁ is O or NH and R₁₂ is —H or —C₁₋₃ alkyl; or
one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, or; ZINC9583892,
or a pharmaceutically acceptable salt thereof, in an amount effective to reduce inflammation in a patient.

A method of treating pulmonary arterial hypertension (PAH) in a patient is provided, comprising administering to the patient a compound having the structure:

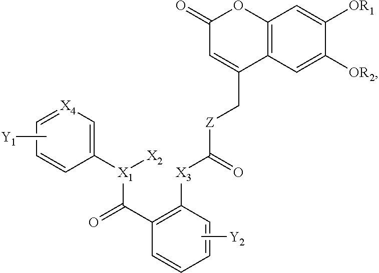

wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H or —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl), —NHR₆ where R₆ is —H or —C₁₋₃ alkyl, —NHC(O)—R₇ where R₇ is —H or —C₁₋₃ alkyl, —OR₈ where Rₐ is —H or —C₁₋₃ alkyl; C₁₋₃ alkoxy), —OC(O)—R₉ where R₉ is —H or —C₁₋₃ alkyl; ester), —C(O)—R₁₀ where R₁₀ is —H or —C₁₋₃ alkyl, or —C(O)—R₁₁—R₁₂ where R₁₁ is O or NH and R₁₂ is —H or —C₁₋₃ alkyl; or
one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, or; ZINC9583892,
or a pharmaceutically acceptable salt thereof, in an amount effective to treat pulmonary arterial hypertension (PAH) in the patient.

A compound is provided, comprising the structure:

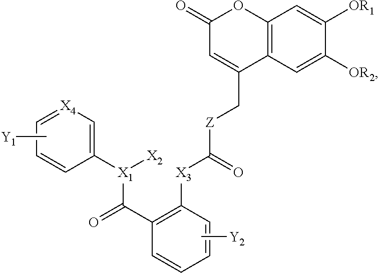

wherein
R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo,, or —NO₂; Y₁ is —H, —C₁₋₃ alkyl, halo, —NO₂, —CN, —CF₃, —SO₂R₄ where R₄ is —OH, —C₁₋₃ alkyl, —NHR₅ where R₅ is H or —C₁₋₃ alkyl, —NHR₆ where R₆ is —H or C₁₋₃ alkyl, —NHC(O)—R₇ where R₇ is —H or —C₁₋₃ alkyl, —OR₈ where R₈ is —H or —C₁₋₃ alkyl), —OC(O)—R₉ where R₉ is —H or —C₁₋₃ alkyl), —C(O)—R₁₀ where R₁₀ is —H or —C₁₋₃ alkyl), or —C(O)—R₁₁—R₁₂ where R₁₁ is O or NH and R₁₂ is —H, —C₁₋₃ alkyl), or a pharmaceutically acceptable salt thereof, excluding MolPort-004-267-958.

A pharmaceutical composition is provided, comprising:
a compound either:
   having the structure:

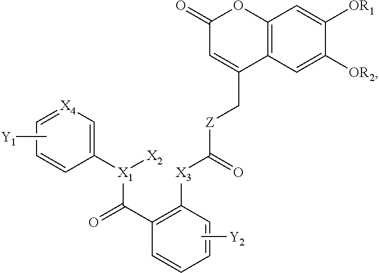

wherein R₁ and R₂ are both or individually (independently) —H or —C₁₋₃ alkyl; Z is O or NH; X₁,X₂,X₃ are, independently N or C; X₄ is ortho, meta or para to X₁ and is N or C; Y₂ is —H, —C₁₋₃ alkyl, halo (—F, —Cl, —Br, or —I), or —NO₂ (nitro); Y₁ is —H, —C₁₋₃ alkyl, halo (—F, —Cl, —Br, or —I), —NO₂, —CN (nitrile), —CF₃ (trifluoromethyl), —SO₂R₄ where R₄ is —OH, —C₁₋₃ alkyl, —NHR₅ where R₅ is H, —C₁₋₃ alkyl; sulfonyl), —NHR₆ (R₆ may be —H, —C₁₋₃ alkyl; amino), —NHC(O)—R₇ (R₇ may be —H, —C₁₋₃ alkyl; amide), —OR₈ (R₈ may be —H, —C₁₋₃ alkyl; C₁₋₃ alkoxy), —OC(O)—R₉ (R₉ may be —H, —C₁₋₃ alkyl; ester), —C(O)—R₁₀ (R₁₀ may be —H, —C₁₋₃ alkyl; aldehyde or keto), or —C(O)—R₁₁-R₁₂ (R₁₁ may be O or NH; R₁₂ may be —H, —C₁₋₃ alkyl; amide or ester), or a pharmaceutically acceptable salt thereof; or
chosen from one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, and ZINC9583892, or a pharmaceutically acceptable salt thereof; and
a pharmaceutically-effective excipient,
wherein the composition comprises an amount of the compound effective to treat or reduce inflammation, cardiovascular inflammation, vascular inflammation (e.g., having vascular endothelium inflammation or a disease having vascular endothelium inflammation as a symptom, such as pulmonary hypertension, restenosis, essential hypertension, atherosclerosis, and stroke), a disease characterized by vascular inflammation, or a disease of innate and acquired immunity, or for treating a coronavirus infection, such as a SARS-CoV-2 infection in a patient, such as a human patient.

Also provided is a method of treating a patient having a C at SNP rs11154337 and having inflammation and/or an inflammatory disease, such as: cardiovascular inflammation or vascular inflammation, or is associated with a disease such as pulmonary hypertension, restenosis, essential hypertension, atherosclerosis, viral infection, bacterial infection, fungal infection, parasite infection, COVID (coronavirus disease), ARDS (acute respiratory distress syndrome), acute lung injury, stroke, neurodegeneration, cancer, an autoimmune disease, or a disease innate and acquired immunity, comprising, using gene editing, such as, for example and without limitation, CRISPR/Cas9- or TALEN-based methods to change one or more C at SNP rs11154337 to a G.

The following provides exemplary, non-limiting aspects, embodiments, or examples of the present invention.

Clause 1. A method of treating inflammation in a patient, such as a human patient, comprising:
administering to the patient an amount of a compound, having the structure:

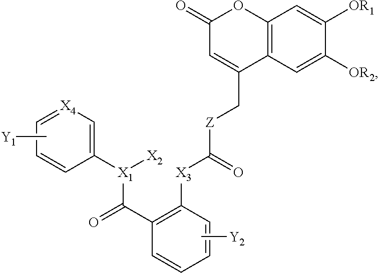

wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl, —NHR₆ where R₆ is —H or —C₁₋₃ alkyl, —NHC(O)—R₇ where R₇ is —H or —C₁₋₃ alkyl, —OR₈ where R₈ is —H or —C₁₋₃ alkyl, —OC(O)—R₉ where R₉ is —H or —C₁₋₃ alkyl, —C(O)—R₁₀ where R₁₀ is —H or —C₁₋₃ alkyl, or —C(O)—R₁₁—R₁₂ where R₁₁ is O or NH and R₁₂ is —H or —C₁₋₃ alkyl; or
one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, or; ZINC9583892,
or a pharmaceutically acceptable salt thereof, in an amount effective to reduce inflammation in a patient.

Clause 2. The method of clause 1, wherein the inflammation is cardiovascular inflammation or vascular inflammation, or is associated with a disease such as pulmonary hypertension, restenosis, essential hypertension, atherosclerosis, viral infection, bacterial infection, fungal infection, parasite infection, COVID (coronavirus disease), ARDS (acute respiratory distress syndrome), acute lung injury, stroke, neurodegeneration, cancer, an autoimmune disease, or a disease innate and acquired immunity.

Clause 3. The method of clause 1, wherein the patient has vascular inflammation.

Clause 4. The method of clause 1, wherein the inflammation is associated with a viral or bacterial infection.

Clause 5. The method of clause 2, wherein the inflammation is associated with a coronavirus infection in the patient, optionally a severe acute respiratory syndrome from a coronavirus infection in the patient.

Clause 6. The method of clause 2, wherein the inflammation is associated with a bacterial infection in the patient.

Clause 7. The method of clause 6, wherein the bacterial infection is a Klebsiella pneumoniae infection in the patient, optionally reducing or preventing lung damage in the patient.

Clause 8. The method of clause 1, wherein the inflammation is associated with one of more of: pulmonary arterial hypertension (PAH), pulmonary hypertension due to left heart disease, pulmonary hypertension due to lung disease, pulmonary hypertension due to chronic blood clots in the lungs, and pulmonary hypertension due to unknown causes.

Clause 9. A method of treating pulmonary arterial hypertension (PAH) in a patient, comprising administering to the patient a compound having the structure:

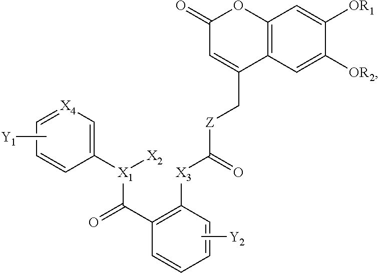

wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H or —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl), —NHR₆ where R₆ is —H or —C₁₋₃ alkyl, —NHC(O)—R₇ where R₇ is —H or —C₁₋₃ alkyl, —OR₈ where Rₐ is —H or —C₁₋₃ alkyl; C₁₋₃ alkoxy), —OC(O)—R₉ where R₉ is —H or —C₁₋₃ alkyl; ester), —C(O)—R₁₀ where R₁₀ is —H or —C₁₋₃ alkyl, or —C(O)—R₁₁—R₁₂ where R₁₁ is O or NH and R₁₂ is —H or —C₁₋₃ alkyl; or
one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, or; ZINC9583892,
or a pharmaceutically acceptable salt thereof, in an amount effective to treat pulmonary arterial hypertension (PAH) in the patient.

Clause 10. The method of any one of clauses 1-9, wherein Z is NH.

Clause 11. The method of any one of clauses 1-10, wherein R₁ and R₂ are, independently, Me or H.

Clause 12. The method of any one of clauses 1-10, wherein R₁ and R₂ are H.

Clause 13. The method of any one of clauses 1-9, wherein the compound is compound 958 (MolPort-004-267-958), or a pharmaceutically acceptable salt thereof.

Clause 14. The method of any one of clauses 1-9, wherein the compound is compound 958ₐₘᵢ, having the exemplary structure:

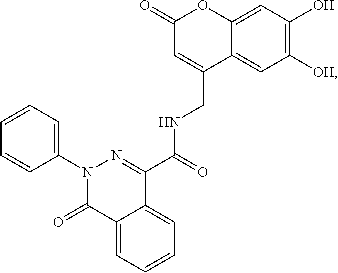 [SMILES: O=C(NCc1cc(=O)oc2cc(O)c(O)cc12)c1nn(-c2ccccc2)c(=O)c2ccccc12]

or a pharmaceutically acceptable salt thereof.

Clause 15. The method of any one of clauses 1-14, wherein the patient is administered an amount of the compound, or a pharmaceutically-acceptable salt thereof, effective to reduce inflammation in the patient or to treat pulmonary hypertension in the patient.

Clause 16. The method of any one of clauses 1-14, comprising administering to the patient from 1 μg to 10 g, or from 1 ng to 100 mg/kg of the compound per day, or to a concentration ranging from 1 to 40 μM in a patient's bodily fluid, e.g. blood, serum, plasma, etc.

Clause 17. The method of any one of clauses 1-16, wherein the patient is heterozygous or homozygous for C at rs11154337.

Clause 18. The method of any one of clauses 1-16, wherein the patient is homozygous for C at rs11154337.

Clause 19. The method 17 or 18, further comprising obtaining genetic data for the patient and determining if the patient has one or two alleles for C at rs11154337.

Clause 20. The method of clause 17 or 18, further comprising determining if the patient has one or two alleles for C at rs11154337.

Clause 21. The method of clause 5, wherein the coronavirus infection is one or more of Middle East Respiratory Syndrome Coronavirus (MERS-CoV), Severe Acute Respiratory Syndrome Coronavirus-2 (SARS-CoV-2), or a disease caused thereby, such as Coronavirus Disease 2019 (COVID-19).

Clause 22. The method of clause 21, wherein the coronavirus is SARS-CoV-2.

Clause 23. The method of clause 1, for reducing infectivity of a coronavirus or a herpesvirus infection in a cell.

Clause 24. A compound, comprising the structure:

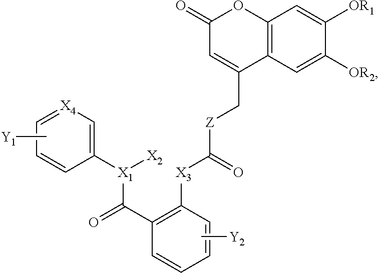

wherein
R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo,, or —NO₂; Y₁ is —H, —C₁₋₃ alkyl, halo, —NO₂, —CN, —CF₃, —SO₂R₄ where R₄ is —OH, —C₁₋₃ alkyl, —NHR₅ where R₅ is H or —C₁₋₃ alkyl, —NHR₆ where R₆ is —H or C₁₋₃ alkyl, —NHC(O)—R₇ where R₇ is —H or —C₁₋₃ alkyl, —OR₈ where R₈ is —H or —C₁₋₃ alkyl), —OC(O)—R₉ where R₉ is —H or —C₁₋₃ alkyl), —C(O)—R₁₀ where R₁₀ is —H or —C₁₋₃ alkyl), or —C(O)—R₁₁—R₁₂ where R₁₁ is O or NH and R₁₂ is —H, —C₁₋₃ alkyl), or a pharmaceutically acceptable salt thereof, excluding MolPort-004-267-958.

Clause 25. The compound of clause 24, wherein Z is NH.

Clause 26. The compound of clause 24 or 25, wherein R₁ and R₂ are, independently, Me or H.

Clause 27. The compound of clause 24 or 25, wherein R₁ and R₂ are H.

Clause 28 The compound of clause 24, having the structure:

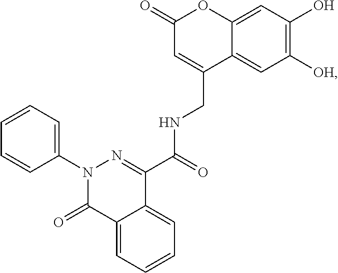 [SMILES: O=C(NCc1cc(=O)oc2cc(O)c(O)cc12)c1nn(-c2ccccc2)c(=O)c2ccccc12]

or a pharmaceutically acceptable salt thereof.

Clause 29. A pharmaceutical composition comprising:
a compound either:
   having the structure:

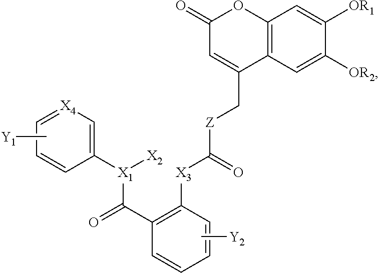

wherein R₁ and R₂ are both or individually (independently) —H or —C₁₋₃ alkyl; Z is O or NH; X₁,X₂,X₃ are, independently N or C; X₄ is ortho, meta or para to X₁ and is N or C; Y₂ is —H, —C₁₋₃ alkyl, halo (—F, —Cl, —Br, or —I), or —NO₂ (nitro); Y₁ is —H, —C₁₋₃ alkyl, halo (—F, —Cl, —Br, or —I), —NO₂, —CN (nitrile), —CF₃ (trifluoromethyl), —SO₂R₄ where R₄ is —OH, —C₁₋₃ alkyl, —NHR₅ where R₅ is H, —C₁₋₃ alkyl; sulfonyl), —NHR₆ (R₆ may be —H, —C₁₋₃ alkyl; amino), —NHC(O)—R₇ (R₇ may be —H, —C₁₋₃ alkyl; amide), —OR₈ (Rₐ may be —H, —C₁₋₃ alkyl; C₁₋₃ alkoxy), —OC(O)—R₉ (R₉ may be —H, —C₁₋₃ alkyl; ester), —C(O)—R₁₀ (R₁₀ may be —H, —C₁₋₃ alkyl; aldehyde or keto), or —C(O)—R₁₁—R₁₂ (R₁₁ may be O or NH; R₁₂ may be —H, —C₁₋₃ alkyl; amide or ester), or a pharmaceutically acceptable salt thereof; or
chosen from one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, and ZINC9583892, or a pharmaceutically acceptable salt thereof; and
a pharmaceutically-effective excipient,
wherein the composition comprises an amount of the compound effective to treat or reduce inflammation, cardiovascular inflammation, vascular inflammation (e.g., having vascular endothelium inflammation or a disease having vascular endothelium inflammation as a symptom, such as pulmonary hypertension, restenosis, essential hypertension, atherosclerosis, and stroke), a disease characterized by vascular inflammation, or a disease of innate and acquired immunity, or for treating a coronavirus infection, such as a SARS-CoV-2 infection in a patient, such as a human patient.

Clause 30. The composition of clause 29, wherein Z is NH.

Clause 31. The composition of clause 29 or 30, wherein R₁ and R₂ are, independently, Me or H.

Clause 32. The composition of clause 29 or 30, wherein R₁ and R₂ are H.

Clause 33. The composition of clause 29, having the structure:

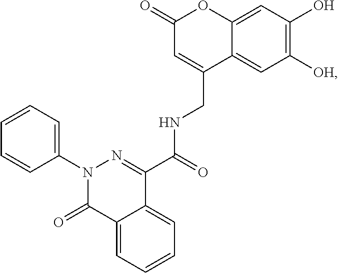 [SMILES: O=C(NCc1cc(=O)oc2cc(O)c(O)cc12)c1nn(-c2ccccc2)c(=O)c2ccccc12]

or a pharmaceutically acceptable salt thereof.

Clause 34. The composition of clause 29, wherein the compound is MolPort-004-267-958.

Clause 35. A method of treating a patient having a C at SNP rs11154337 and having inflammation and/or an inflammatory disease, such as: cardiovascular inflammation or vascular inflammation, or is associated with a disease such as pulmonary hypertension, restenosis, essential hypertension, atherosclerosis, viral infection, bacterial infection, fungal infection, parasite infection, COVID (coronavirus disease), ARDS (acute respiratory distress syndrome), acute lung injury, stroke, neurodegeneration, cancer, an autoimmune disease, or a disease innate and acquired immunity, comprising, using gene editing, such as, for example and without limitation, CRISPR/Cas9- or TALEN-based methods to change one or more C at SNP rs11154337 to a G.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. An exemplary nucleic acid sequence (SEQ ID NO: 1).

FIG. 2. An exemplary nucleic acid sequence of rs11154337 C allele (top strand, SEQ ID NO: 30).

FIG. 3. An exemplary sequence for human NCOA7 mRNA (SEQ ID NO: 2).

FIG. 4. Convergent inflammatory regulation of NCOA7 across cellular, animal, and human instances of PAH. (A) Transcriptomic analysis of human PAECs under control or IL-1β (N=3/group). Z-score presented as positive in blue and negative in gold. Genes listed have an FDR-corrected P-value<0.05. (B and C) NCOA7 isoform expression via RT-qPCR (N=3/group). (D and F) Immunofluorescent (IF) staining for and (E and G) quantification of NCOA7 (red), CD31⁺ ECs (green), α-SMA⁺ smooth muscle cells (white), and DAPI-stained nuclei (blue) in pulmonary vessels of mouse (N=8/group) and human PH models (N=6-7/group). (H and I) violin plots of ECs expressing NCOA7 identified via single-cell RNA sequencing from lungs of control or PAH patients (N>3/group). Cells were identified as expressing NCOA7if the transformed expression value was >0.2. (J and K) NCOA7 isoform expression under RNAi against RELA (N=3/group). Two-way ANOVA. (L) ChIP-qPCR against p65/ReIA binding to full- and short-length promoter regions (N=3/group). All data are analyzed by Student's t-test unless otherwise specified and presented as mean±standard deviation.

FIGS. 5A and 5B. Inflammatory regulation of NCOA7 and in vitro tools to modulate its expression. (A) Structure of the full-length and short-length isoforms of NCOA7 located at 6q22.33. Exons are denoted by rectangles. The black rectangle denotes the first exon of the short-length isoform. (B to E) NCOA7 isoform expression via RT-qPCR in human PAECs (N=3/group). (F and G) Ncoa7 isoform expression via RT-qPCR in CD31⁺ isolated cells from total lung of IL-6 transgenic mice under chronic hypoxia. (N=5-7/group). (H to K) Immunofluorescent staining for and quantification of NCOA7 (red), CD31⁺ ECs (green), α-SMA⁺ smooth muscle cells (white), and DAPI-stained nuclei (blue) in pulmonary vessels of mouse (N=5/group) and rat PH models (N=5/group). (L to N) NCOA7 isoform expression via RT-qPCR in HPAECs (N=3/group). (O and P) Immunoblot and densitometry of Myc-tagged NCOA7 isoforms (N=3/group). All data are analyzed by Student's t-test unless otherwise specified and presented as mean±standard deviation.

FIGS. 6A-6C. NCOA7 deficiency results in lysosomal dysfunction and lipid accumulation under proinflammatory conditions. A) Transcriptomic analysis of PAECs under IL-1β subjected to RNAi against control or NCOA7 (N=3/group). Z-score presented as positive in blue and negative in gold. Identified lysosomal genes have an FDR-corrected P-value<0.05. (B) Expression of ATP6V1B2 under siNC or siNCOA7 via RT-qPCR (N=3/group). (C) Expression of ATP6V1B2 with lentiviral delivery of control (LV-GFP), NCOA7ₛₕₒᵣₜ, or NCOA7_{full} isoforms (N=3/group). Data analyzed by one-way ANOVA. (D and E) Association of the V-ATPase subunit ATP6V1B2 with NCOA7 measured by proximity ligation assay (orange). Bottom panel demonstrates control images of ATP6V1B2, NCOA7, or neither antibody. Top panel demonstrates dual incubation of ATP6V1B2 and NCOA7 antibodies with lentiviral transduction of GFP control, NCOA7ₛₕₒᵣₜ, or NCOA7_{full}. Quantification of amplified signal per cell (N=5 cells/group). Data analyzed by one-way ANOVA. (F and G) LysoLive probe (green) reflecting β-galactosidase activity and thus lysosomal acidification. Quantified as fluorescence per cell (N=10 cells/group). (H and I) Silicone rhodamine (SiR)-Lysosome dye targeting active cathepsin D (purple) and indicating low lysosomal pH. Quantified as fluorescence per cell (N=10 cells/group). (J) Live-cell confocal microscopy of lysosomes (LysoTracker Deep Red) and lysosomal acidification (LysoSensor Green DND-189). (K) Median fluorescence intensity (MFI) ratio of LysoSensor Yellow/Blue DND-160 dye via flow cytometry representing lysosomal acidification (N=3/group). (L and M) Representative images and quantification of lysosomal area in transmission electron micrographs (N=20/group). Yellow arrows indicate lamellar-like inclusions reflecting lipid accumulation within lysosomes. (N and O) IF staining of colocalization (yellow) of neutral sterols (BOPIDY in green) and acidic lysosomes (LysoTracker in red). Colocalization was measured using EzColocalization and plotted as Pearson's Correlation Coefficient (N=15 cells/group). All data are analyzed by Two-way ANOVA unless otherwise specified and presented as mean±standard deviation.

FIG. 7. NCOA7 binding partner ATP6V1B2 is similarly upregulated in inflammatory models of PH. (A to H) Immunofluorescent staining for and quantification of ATP6V1B2 (red), CD31⁺ ECs (green), α-SMA⁺ smooth muscle cells (white), and DAPI-stained nuclei (blue) in pulmonary vessels of rodent PH models (N=5-7/group) and humans with PAH (N=6-7/group). All data are analyzed by Student's t-test and presented as mean±standard deviation.

FIG. 8A-8B. NCOA7 deficiency reprograms sterol metabolism to upregulate oxysterols and bile acids. (A) Transcriptomic analysis of PAECs under IL-1β subjected to RNAi against control or NCOA7 (N=3/group). Z-score presented as positive in blue and negative in gold. Identified cholesterol metabolism genes have an FDR-corrected P-value<0.05. (B) Gene set enrichment analysis of top 15 pathways by FDR-adjusted p-value with a majority related to sterol metabolism and homeostasis (highlighted with red arrows). (C) Expression of LDLR via RT-qPCR (N=3/group). (D and E) Flow cytometric analysis of fluorescently tagged NBD-cholesterol uptake (N=3/group). (F) Total cholesterol content as measured by relative luminescence under siNC or siNCOA7 or (G) with overexpression using lentiviral GFP, NCOA7ₛₕₒᵣₜ, or NCOA7_{full}. Data analyzed by one-way ANOVA (N=6/group). (H) Expression of CH25H via RT-qPCR (N=3/group). (I to N) IF staining for and quantification of CH25H (red), CD31⁺ ECs (green), α-SMA⁺ smooth muscle cells (white), and DAPI-stained nuclei (blue) in the pulmonary vessels of mouse (N=8/group), rats (N=5/group), and human (N=6-7/group) PH models. Student's t-test. (O to Q) Targeted oxysterol quantification via LC-MS (N=5/group) and (R to W) unbiased bile acid metabolite quantification via LC-MS organized by proposed pathways in shaded boxes (N=4-6/group). The metabolite 7HOCA is depicted in panel (W). All data are analyzed by Two-way ANOVA unless otherwise specified and presented as mean±standard deviation.

FIGS. 9A and 9B. NCOA7 modulation of oxysterol and bile acid metabolism is not dependent on sterol flux in the de novo synthesis of cholesterol. (A to N) Direct measurement of post-squalene intermediates via liquid chromatography-mass spectrometry (N=5/group). (O and P) Immunofluorescent staining for and quantification of CH25H (red), CD31⁺ ECs (green), α-SMA⁺ smooth muscle cells (white), and DAPI-stained nuclei (blue) in pulmonary vessels of mouse (N=5/group; Student's t-test.). All data are analyzed by two-way ANOVA unless otherwise specified and presented as mean±standard deviation.

FIGS. 10A-10C. The NCOA7—CH25H axis drives pulmonary endothelial immunoactivation. (A to F) VCAM1 expression via RT-qPCR and immunoblot under (A and B) RNAi against NCOA7, (C and D) lentiviral delivery of NCOA7ₛₕₒᵣₜ or NCOA7_{full}, or (E and F) RNAi against NCOA7 and CH25H (N=3/group). Immune cell adhesion of leukocytes (G to 1) or monocytes (J to L) to an endothelial monolayer (N=6/group). (M and N) Expression of VCAM1 by RT-qPCR and immunoblot in PAECs treated with ethanol control versus 7HOCA (50 μM) for 24 hours (N=3/group; Student's t-test). (O and P) Leukocyte and monocyte adhesion in 7HOCA-treated PAECs compared to ethanol controls (N=6/group; Student's t-test). All data are analyzed by Two-way ANOVA unless otherwise specified and presented as mean±standard deviation.

FIGS. 11A-11C. NCOA7 modulates endothelial cell apoptosis and proliferation and oxysterols immunoactivated the endothelium. (A to C) Apoptosis via caspase-3/7 activity (N=6/group; Two-way ANOVA). (D to E) Proliferation via BrdU incorporation (N=6/group; Two-way ANOVA). (G to O) VCAM1 expression via RT-qPCR (N=3/group) and leukocyte or monocyte adhesion to a monolayer treated with ethanol versus 25HC (25 μM) or triol (5 μM) or tetrol (50 μM) for 24 hours (N=6/group). (P to S) Immunoblot of VCAM1 in PAECs in triplicate under various conditions (red box denotes presentation in main figures). All data are analyzed by Student's t-test unless otherwise specified and presented as mean±standard deviation.

FIGS. 12A and 12B. The G allele of SNP rs11154337 prevents lysosomal lipid accumulation and attenuates oxysterol-mediated immunoactivation in iPSC-ECs. (A to C) Allelic variants of SNP rs11154337 and their clinical readouts of 6MWD and survival in PAH patients in the UPMC cohort (N=93) and STRIDE cohort (N=630). (D) Schematic of iPSC-EC production. (E and F) NCOA7 isoform expression via RT-qPCR (N=3/group). (G and H) SiR-Lysosome dye against active cathepsin D (purple; N=10 cells/group). (I) ATP6V1B2 expression via RT-qPCR (N=3/group). (J) Transmission electron micrograph quantification of lysosomal area. (K) Total cholesterol content as measured by relative luminescence (N=6/group). (L and M) BODIPY dye against neutral lipids (green; N=10 cells/group). (N) CH25H expression via RT-qPCR (N=3/group). (O) Targeted 25HC quantification via LC-MS (N=5/group). (P and Q) VCAM1 expression via RT-qPCR or immunoblot (N=3/group). (R and S) Leukocyte and monocyte adhesion to iPSC-EC monolayer (N=6/group). All data are analyzed by Two-way ANOVA unless otherwise specified and presented as mean±standard deviation.

FIG. 13. Genetic loss of Ncoa7 does not alter left ventricular function and upregulates plasma oxysterols and bile acids. (A to E) Echocardiographic measurements of heart rate, left ventricular fractional shortening (LVFS), left ventricular ejection fraction (LVEF), and left ventricular posterior wall distance during diastole and systole (LVPW;d and LVPW;s) in (A to E) Ncoa7^{-/-}×Il6 Tg⁺ mice (N=3-5/group) and (J to N) PBS or 7HOCA (10 mg/kg) mice under four weeks of hypoxia (N=3-6/group). (F to 1) Measurement of 7HOCA using LC-MS in the serum of Il6 Tg⁺ versus Ncoa7^{-/-×Il}6 Tg⁺ mice (N=5-8/group). All data are analyzed by Student's t-test unless otherwise specified and presented as mean±standard deviation.

FIG. 14. Genetic loss of NCOA7 and the orotracheal delivery of 7HOCA worsens PAH in vivo. (A) Ncoa7-null mice crossed onto the Il6 Tg+ PAH model. (B to F) Pulmonary vessels from Il6 Tg+ versus Ncoa7−/−×Il6 Tg+ mice stained for a target protein (i.e., CH25H, VCAM1, or CD11 b; red), the endothelial layer (CD31; green), the smooth muscle layer (α-SMA; white), and nuclear counterstain (DAPI; blue). Quantification of the relative intensity of CH25H or VCAM1, the number of CD11b+ cells per vessel, or the degree of vessel muscularization defined by α-SMA layer thickness to total vessel diameter. N=8/group. (G) Measurement of 7HOCA using LC-MS in the serum of Il6 Tg+ versus Ncoa7−/−×Il6 Tg+ mice. N=5−8/group. (H) Fulton's Index and (I) RVSP of Il6 Tg+ versus Ncoa7−/−×Il6 Tg+ mice. N=6−10/group. (J) Mice received orotracheal delivery of either PBS or 7HOCA (10 mg/kg) for four weeks under hypoxic (10% O₂) conditions and sacrificed at 16 weeks. (K to N) Pulmonary vessels from PBS versus 7HOCA mice stained for a target protein (i.e., VCAM1, or CD11b; red), the endothelial layer (CD31; green), the smooth muscle layer (α-SMA; white), and nuclear counterstain (DAPI; blue). Quantification of the relative intensity of VCAM, the number of CD11 b+ cells per vessel, or the degree of vessel muscularization defined by α-SMA layer thickness to total vessel diameter. N=4/group. (0) Fulton's Index and (P) RVSP of mice receiving orotracheal PBS or 7HOCA. N=4−7/group. All data are analyzed by Student's t-test unless otherwise specified and presented as mean±standard deviation.

FIGS. 15A and 15B. Genomic architecture of NCOA7 and the creation of SNP-edited iPSC-derived ECs. (A) High-throughput chromatin conformation capture on human umbilical vein endothelial cells (GEO IDs GSM3438650 and GSM3438651). Blue bars represent the bias-removed chromatin interaction frequency, and the purple dots represent the distance-normalized interaction frequency. The arcs, depicted in indigo, represent the identified interactions of SNP rs11154337 in red defined by the threshold line in green. Data were obtained from the 3D-genome Interaction Viewer & database (3 DIV; 3 div.kr). Gene map of NCOA7 isoforms depicted below. (B) 3C assay in human PAECs predicting an interaction between the 3′ end of restriction enzyme (BspHI) digested genomic DNA fragment containing the promoter of NCOA7 (N3) and the 5′ end of genomic DNA fragment containing SNP rs11154337 (S5) that produces a fusion sequence ligated at the BspHI cutting site (N3S5). DNA gel confirming the existence of a 107 bp PCR product with primers targeting the fusion sequence across the BspHI cutting site. The non-ligated genomic DNA was used as control for PCR. (C) DNA sequencing of PCR product to confirm the N3S5 fusion sequence. (D) ChIP-qPCR of p65/ReIA binding to SNP rs11154337 region (N=3/group). (E) Schematic of cohorts utilized. (F) DNA sequencing of SNP rs11154337 in CRISPR-Cas9, SNP-edited iPSCs. (G) Flow cytometry on sorting efficiency of iPSC-derived ECs via MACS. (H) iPSC-EC morphology by brightfield, functional capacity by vessel formation, and immunofluorescent staining of EC-specific markers CD144 (green) and CD31 (red). (I) Transmission electron microscopy of iPSC-ECs. (J) Immunoblot of VCAM1 in iPSC-ECs in triplicate (red box denotes presentation in main figures). All data are analyzed by Student's t-test unless otherwise specified and presented as mean±standard deviation.

FIGS. 16A-16C. Computational modeling identifies 958ₐₘᵢ as a novel NCOA7 activator that abrogates endothelial immunoactivation and PAH. (A to C) Computational protocol for identifying small molecule modulators of NCOA7, comprised of three major components: (A) druggability simulations, (B) pharmacophore modeling, and (C) virtual screening. (D and E) Refinement of the identified compound 958 after MD simulations into its analogue 958ₐₘᵢ. Compound atoms and NCOA7 residues interactions are shown in two dimensions. Stronger interactions are shown in orange dashed lines, while weaker interactions are shown in gray dashed lines. Orange solid lines are strong/persistent interactions with more than 0.3 μs cumulative duration per interaction over 0.6 μs total simulations. (F and G) Association of the V-ATPase subunit ATP6V1B2 with NCOA7 measured by proximity ligation assay (orange). Panels demonstrate dual incubation of ATP6V1B2 and NCOA7 antibodies with DMSO or 958ₐₘᵢ. Quantification of amplified signal per cell (N=15 cells/group). (H) CH25H expression via RT-qPCR (N=3/group; Two-way ANOVA). (I and J) Expression of VCAM1 by RT-qPCR and immunoblot in PAECs treated with DMSO versus 958ₐₘᵢ (20 μM) for 24 hours (N=3/group; Two-way ANOVA). (K and L) Leukocyte and monocyte adhesion in 958ₐₘᵢ-treated PAECs compared to DMSO control (N=6/group. (M) Rats were loaded with monocrotaline (80 mg/kg i.p.) one week before initiation of a daily dose of 958ₐₘᵢ (7.5 mg/kg i.p.) for 10 days. (N to R) Pulmonary vessels from DMSO versus 958ₐₘᵢ injected rats stained for a target protein (i.e., CH25H, VCAM1, or CD11b; red), the endothelial layer (CD31; green), the smooth muscle layer (α-SMA; white), and nuclear counterstain (DAPI; blue). Quantification of the relative intensity of CH25H or VCAM1, the number of CD11b⁺ cells per vessel, or the degree of vessel muscularization defined by α-SMA layer thickness to total vessel diameter. N=6-7/group. (S) Fulton's Index and (T) RVSP of monocrotaline rats receiving intraperitoneal DMSO or 958ₐₘᵢ. N=5-15/group. All data are analyzed by Student's t-test unless otherwise specified and presented as mean±standard deviation.

FIG. 17. Gaussian network model analysis of NCOA7 structural dynamics. (A) Mobility profiles of residues in the most collective three modes of motion of NCOA7 catalytic domain. Dominant hinge residues for each mode are indicated by arrows and labeled. (B) Color-coded structures with regions exhibiting largest conformational flexibility are colored in red and minimal flexibility are colored in blue for the three most cooperative modes. Hinge residues for each mode are shown in spheres and labeled.

FIG. 18. Contact duration of the binding of compounds 958 and 958ₐₘᵢ to NCOA7 observed in molecular dynamics simulations. (A) Contact duration for NCOA7 residues from MD simulations (three independent runs, each of 0.2 ms; total 0.6 μs for each compound). 958 is shown in cyan filled-squares and 958ₐₘᵢ in magenta filled-squares. Contact is defined as being at a cutoff distance of 4.0 Å between any heavy atoms of the residue and the compound. (B) 2D structures of 958 and 958ₐₘᵢ with atom ID numbers. (C) Nine graphs for contact duration between the specific residues of NCOA7 (listed in X-axis in A) and the compound atoms.

FIG. 19. Binding affinities observed in MD simulations of NCOA7 complexed with 958 and 958ₐₘᵢ. (A) Time evolution of binding affinities for the two compounds on the left panels, referring to three independent runs for each system. The histograms on the right are obtained by compiling the snapshots from all three runs for each compound. The average binding affinities (in kcal/mol) and corresponding standard deviations (over the complete duration of the runs) are indicated in each case. (B) Binding poses of 958ₐₘᵢ in respective run 2 and run 3 at 200 ns.

FIG. 20. 958ₐₘᵢ does not alter left ventricular function nor induces hepatic or renal toxicity in rats. (A) Immunoblot of VCAM1 in human PAECs treated with DMSO or 958ₐₘᵢ in triplicate (red box denotes presentation in main figures). (B to F) Echocardiographic measurements of heart rate, left ventricular fractional shortening (LVFS), left ventricular ejection fraction (LVEF), and left ventricular posterior wall distance during diastole and systole (LVPW;d and LVPW;s) in monocrotaline rats treated with DMSO or 958ₐₘᵢ (7.5 mg/kg i.p.) (N=3-4/group). (G and H) Heart rate and mean arterial pressure (MAP) during right heart catheterization (N=6-15/group). (I to K) Expression of Gpt and Got1 in hepatic tissue and Cst3 in renal tissue of rats treated with DMSO or 958ₐₘᵢ (N=5-6/group). All data are analyzed by Student's t-test unless otherwise specified and presented as mean±standard deviation.

FIG. 21. SNP rs11154337 and 958 modulate the entry of various pseudotyped envelope viruses. (A to D) iPSCs transfected with human ACE2 receptor demonstrate decreased entry of multiple pseudotyped coronaviruses and a herpesvirus with the presence of the G allele at SNP rs11154337, which confers increased NCOA7 expression. (E) Application of the NCOA7 activator 958 enhances the activity of NCOA7 to prevent pseudotyped SARS-CoV-2 infection (D614G Spike) in HEK293 cells transfected with human ACE2 receptor. (F and G) 958 parental (2 μM) and 958ₐₘᵢ (5 μM) decrease BA.2 spike pseudotyped virus infection in iPSCs transfected with the human ACE2 receptor. (H and 1) 958 parental (2 μM) and 958ₐₘᵢ (5 μM) decrease D614G spike pseudotyped virus infection in iPSCs transfected with the human ACE2 receptor. All data are analyzed by Student's t-test unless otherwise specified and presented as mean±standard deviation.

FIG. 22. Coronavirus-infected human ACE2 transgenic mice treated with the NCOA7 activator 958 have decreased lung inflammation and mortality. (A and B) Human ACE2 transgenic mice infected with two different coronavirus strains have significantly improved mortality when treated with 958. (C to H) Mouse lung tissue demonstrates decreased viral load and attenuation of various proinflammatory markers (IL-1α, IL-1β, IFN-γ, VCAM1, and ICAM1) when treated with 958 via RT-qPCR. All data are analyzed by Student's t-test unless otherwise specified and presented as mean±standard deviation.

FIG. 23. Mice infected with Klebsiella pneumoniae demonstrate improvement of acute lung injury when treated with the NCOA7 activator 958. (A and B) Representative images of lung tissue sections stained with H&E after 48 hours of intratracheal infection with Klebsiella pneumoniae in mice treated with DMSO Comp. 958. The black arrow represents neutrophils, while the red arrowhead represents alveolar edema (400+ original magnification, 50 μm scale bar). Mice treated with 958 have decreased acute lung injury. (C to E) ELISA on lung homogenate of proinflammatory cytokines like IL-1β, TNF-α, and IL-6. All data are analyzed by Student's t-test unless otherwise specified and presented as mean±standard deviation.

FIG. 24. Loss of NCOA7worsens survival in a mouse model of ischemic stroke. A. Wildtype versus Ncoa7-deficient mice were subjected to an experimental model of ischemic stroke. Mice received either a sham surgery or transient middle cerebral artery occlusion (tMCAO) for 60 minutes before restoration of cerebral blood flow. Mice were then collected at 24 and 48 hours for further study. B. NCOA7 knockout mice have significantly decreased survival at 24 and 48 hours post-tMCAO compared to wildtype controls. There was no appreciable change in body weight at 24 or 48 hours post-tMCAO. C. Using Laser Doppler Flowmetry, regional cerebral blood flow (rCBF) was quantified in the contralateral (CL) and ipsilateral (IL) cerebral cortices of mice. Mice deficient for NCOA7 had increased rCBF 24 hours post-tMCAO in the IL cerebral cortex compared to wildtype controls. All data are analyzed as Student's t-test (*P<0.05, **P<0.01) and presented as mean+/−standard deviation.

FIG. 25. Loss of NCOA7increases infarct volume and capillary leak in mice post-ischemic stroke. A. Mouse brain tissue was sectioned in 30 microns before immunofluorescent staining. Brain sections were stained with the neuronal marker microtubule-associated protein 2 (MAP2, green) to assess for infarct volume post-tMCAO. In mice deficient for NCOA7, there is significant increase in infarct volume after ischemic stroke, indicating greater neuronal cell death. There was no appreciable change in tissue swelling compared to wildtype controls. B. To assess capillary leakage, brain sections were stained for the plasma protein albumin (red). Notably, mice deficient for NCOA7 had greater leakage of albumin into brain tissue post-tMCAO, indicating greater damage to the brain microvasculature as compared to wildtype controls. All data are analyzed as Student's t-test (*P<0.05, **P<0.01) and presented as mean+/−standard deviation.

FIG. 26. Loss of NCOA7 results in worsened neuroinflammation after ischemic stroke. A. Mouse brain sections were stained using an immunofluorescent protocol. Brains were stained with the astrocytic marker glial fibrillary acidic protein (GFAP, green), the microglial marker ionized calcium binding adaptor molecule 1 (IBA1, red), and a nuclear marker (DAPI, blue). Images were obtained using confocal microscopy in both the cortex (Ctx) and striatum (Str) of wildtype and NCOA7-knockout mice after tMCAO. Mice deficient for NCOA7 have markedly worse neuroinflammation as noted by swelling of the astrocytic processes (green) in both cortical and striatal tissue. In addition, microglia (red) are hypertrophic and elongated in knockout mice as compared to wildtype controls. These data indicate substantial reactive gliosis and neuroinflammation in brains deficient for Ncoa7 post-tMCAO.

FIG. 27. Loss of NCOA7 results in significant hypermyelination in a mouse model of ischemic stroke. A. Brain sections were stained with myelin basic protein (MBP, green) using an immunofluorescent protocol. NCOA7-deficient mice had significantly enhanced myelination globally, especially at the levels of the corpus callosum (CC) and the external capsule (EC). Notably, there was no appreciable difference in comparison of contralateral (CL) versus ipsilateral (IL) hemispheres post-tMCAO. All data are analyzed as Student's t-test (*P<0.05, **P<0.01) and presented as mean+/−standard deviation.

FIGS. 28A-28E provide exemplary structures for various compounds described herein.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise” or “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of inflammation, cardiovascular inflammation, vascular inflammation (e.g., endothelial inflammation), a disease characterized by vascular inflammation, or a disease of innate and acquired immunity means administration to a patient by any suitable dosage regimen, procedure and/or administration route of a composition, device, or structure with the object of achieving a desirable clinical/medical end-point, including but not limited to, for PAH, a mean pulmonary artery pressure >25 mmHg measured by right heart catheterization supine at rest. Reducing or preventing further development of vascular endothelial inflammation, e.g., PAH. An amount of any reagent or therapeutic agent, administered by any suitable route, effective to treat a patient is an amount capable of preventing, reducing, and/or eliminating endothelial inflammation, such as PAH and/or reducing the severity of one or more symptoms of the endothelial inflammation, such as PAH, for example, a mean pulmonary artery pressure <25 mmHg measured by right heart catheterization supine at rest. The therapeutically-effective amount of each therapeutic may range from 1 pg per dose to 10 g per dose, including any amount there between, such as, without limitation, 1 ng, 1 μg, 1 mg, 10 mg, 100 mg, or 1 g per dose. The therapeutic agent may be administered by any effective route, and, for example, as a single dose or bolus, at regular or irregular intervals, in amounts and intervals as dictated by any clinical parameter of a patient, or continuously.

Cardiovascular inflammation, such as vascular inflammation (e.g., endothelial inflammation or vascular endothelial inflammation) may not only be associated with PAH, but with other diseases, including, without limitation: peripheral artery disease, vasculitis including large-, medium-, and small-vessel vasculitis, infectious disease, chronic vascular inflammatory disease, such as atherosclerosis, inflammatory or inflammation-associated conditions, such as restenosis, essential hypertension, and stroke (see, e.g., McLaughlin V V, McGoon M D. Pulmonary arterial hypertension. Circulation. 2006 Sep. 26; 114(13):1417-31). Other diseases that may be effectively treated methods described herein include diseases of innate and acquired immunity, such as heart failure (HFpEF and HFrEF), myocarditis, and atrial fibrillation where inflammatory myeloid cells appear to also worsen symptoms and severity of disease. Pulmonary hypertension may be effectively treated by the compositions and methods described herein, including PAH, pulmonary hypertension due to left heart disease, pulmonary hypertension due to lung disease, pulmonary hypertension due to chronic blood clots in the lungs, and pulmonary hypertension due to unknown causes. Other diseases of innate and acquired immunity include, for example, heart disease, lung disease, sepsis, cancer, and neurodegeneration, e.g., inflammation associated with, for example, heart disease, lung disease, sepsis, cancer, and neurodegeneration.

As used herein, the “treatment” or “treating” of a coronavirus infection means administration to a patient by any suitable dosage regimen, procedure and/or administration route of a composition, device, or structure with the object of achieving a desirable clinical/medical end-point, including but not limited to, for a coronavirus infection, reducing or preventing further development of the coronavirus infection, e.g., as determined below. An amount of any reagent or therapeutic agent, administered by any suitable route, effective to treat a patient is an amount capable of preventing, reducing, and/or eliminating the coronavirus infection and/or reducing the severity of one or more symptoms of the coronavirus infection, for example, fever or chills, cough, shortness of breath or difficulty breathing, fatigue, muscle or body aches, headache, loss of taste or smell, sore throat, congestion or runny nose, nausea or vomiting, or diarrhea. The therapeutically-effective amount of each therapeutic, e.g., compound (1) described below, exemplified by 958 and 958ₐₘᵢ, may range from 1 μg per dose to 10 g per dose, including any amount there between, such as, without limitation, 1 ng, 1 μg, 1 mg, 10 mg, 100 mg, or 1 g per dose. The therapeutic agent may be administered by any effective route, and, for example, as a single dose or bolus, at regular or irregular intervals, in amounts and intervals as dictated by any clinical parameter of a patient, or continuously.

Active ingredients, such as compound (1) described below, exemplified by 958 and 958ₐₘᵢ, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include, for example and without limitation: intravenous, intramuscular, intraocular, or intraperitoneal solutions, oral tablets or liquids, topical ointments or creams and transdermal devices (e.g., patches). In one embodiment, the compound is a sterile solution comprising the active ingredient (drug or compound), and a solvent, such as water, saline, lactated Ringer's solution, or phosphate-buffered saline (PBS). Additional excipients, such as polyethylene glycol, emulsifiers, salts and buffers may be included in the solution.

Suitable dosage forms may include single-dose, or multiple-dose vials or other containers, such as medical syringes or droppers, containing a composition comprising an active ingredient useful for treatment of a coronavirus infection as described herein.

Pharmaceutical formulations adapted for administration include aqueous and non-aqueous sterile solutions which may contain, in addition to the active pharmaceutical ingredient or drug, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An amount effective to treat inflammation or an inflammatory condition in a patient may be 1 μg to 10 g, or from 1 ng to 100 mg/kg of compound 958 per day, for example an amount to produce 20 μM±99%, 90%, 80%, 70%, 60%, 50%, 40%, 30%, 25%, 20%, 15%, 10%, 0.5%, 1%, e.g., from 1 to 40 μM, or any increment therebetween of the compound in a patient's bodily fluid, e.g. blood, serum, plasma, etc.

Viral-associated inflammation, e.g., respiratory inflammation, also may be treated using a compound as described herein. For example, coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause Middle East Respiratory Syndrome (MERS), Severe Acute Respiratory Syndrome (SARS), or Coronavirus Disease 2019 (COVID-19). In cows and pigs, coronaviruses cause diarrhea, while in mice they cause hepatitis and encephalomyelitis.

SARS-CoV-2 is the virus that causes COVID-19, the respiratory illness responsible for the COVID-19 pandemic. SARS-CoV-2 is a positive-sense single-stranded RNA virus that is contagious in humans. Each SARS-CoV-2 virion is approximately 50-200 nanometers in diameter. SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein is the protein responsible for allowing the virus to attach to and fuse with the membrane of a host cell; specifically, its S1 subunit catalyzes attachment, the S2 subunit fusion. Symptoms of SARS-CoV-2 infection may appear 2-14 days after exposure to the virus. Symptoms of SARS-CoV-2 may include, but not limited to, fever or chills, cough, shortness of breath or difficulty breathing, fatigue, muscle or body aches, headache, loss of taste or smell, sore throat, congestion or runny nose, nausea or vomiting, or diarrhea.

As demonstrated below, compounds 958 and 958ₐₘᵢ are shown to be useful in treating inflammation and inflammatory diseases, cardiovascular inflammation, vascular inflammation (e.g., having vascular endothelium inflammation or a disease having vascular endothelium inflammation as a symptom, such as pulmonary arterial hypertension, restenosis, essential hypertension, atherosclerosis, and stroke), a disease characterized by vascular inflammation, or a disease of innate and acquired immunity, such as heart disease, lung disease, sepsis, cancer, and neurodegeneration, e.g., inflammation associated with, for example, heart disease, lung disease, sepsis, cancer, and neurodegeneration. The compound may be used to treat a viral infection, e.g. inflammation associated with a viral infection, such as SARS-CoV-2 or other SARS or MERS virus infections. Compound 958, for example, and without limitation, MolPort-004-267-958, may be described as 6,7-dihydroxy-2-oxo-2H-chromen-4-yl)methyl 4-oxo-3-phenyl3,4-dihydrophthalazine-1-carboxylate, and may have the structure:

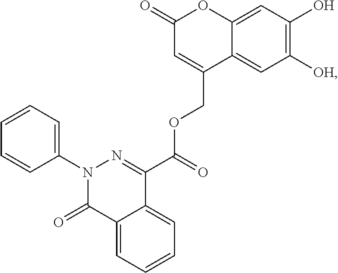 [SMILES: O=C(OCc1cc(=O)oc2cc(O)c(O)cc12)c1nn(-c2ccccc2)c(=O)c2ccccc12]

and includes pharmaceutically acceptable salts thereof, and may include equivalent derivative compounds, such as esters or prodrugs thereof.

Compound 958ₐₘᵢ has the structure:

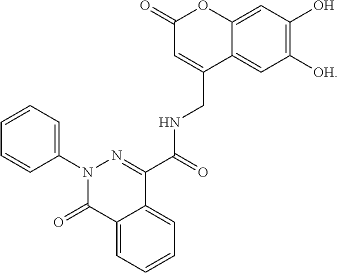 [SMILES: O=C(NCc1cc(=O)oc2cc(O)c(O)cc12)c1nn(-c2ccccc2)c(=O)c2ccccc12]

Compounds 958 and 958ₐₘᵢ are exemplary of a compound (1), provided herein, having the general structure as follows to which similar activity is expected:

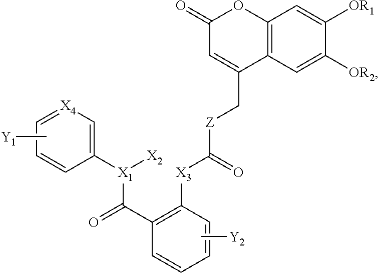

wherein R₁ and R₂ are both or individually (independently) —H or —C₁₋₃ alkyl;
Z is O or NH;
X₁,X₂,X₃ are, independently N or C;
X₄ is ortho, meta or para to X₁ and is N or C;
Y₂ is —H, —C₁₋₃ alkyl, halo (—F, —Cl, —Br, or —I), or —NO₂ (nitro);
Y₁ is —H, —C₁₋₃ alkyl, halo (—F, —Cl, —Br, or —I), —NO₂, —CN (nitrile), —CF₃ (trifluoromethyl), —SO₂R₄ where R₄ is —OH, —C₁₋₃ alkyl, —NHR₅ where R₅ is H, —C₁₋₃ alkyl, —NHR₆ where R₆ may be —H, —C₁₋₃ alkyl, —NHC(O)—R₇ where R₇ may be —H, —C₁₋₃ alkyl, —OR₈ where R₃ may be —H, —C₁₋₃ alkyl, —OC(O)—R₉ where R₉ may be —H, —C₁₋₃ alkyl, or C₁₋₃ alkoxy, —C(O)—R₁₀ where R₁₀ may be —H, —C₁₋₃ alky), or —C(O)—R₁₁—R₁₂ (R₁₁ may be O or NH; R₁₂ may be —H, —C₁₋₃ alkyl), and includes pharmaceutically acceptable salts thereof, and may include equivalent derivative compounds, such as esters or prodrugs thereof. Where an “R group” is an ester or amide, the ester or amide may form a bond linking the active moiety to another moiety, such as an inactive moiety or a carrier. As used herein, “moiety” refers to a portion of a molecule, such as a portion to which activity or functionality may be attributed.

As used herein, “alkyl” refers to straight, branched chain, or cyclic hydrocarbon (hydrocarbyl) groups including, for example, from 1 to about 24 carbon atoms, for example and without limitation C₁₋₃ groups comprising 1, 2, or 3 carbons, e.g., methyl, ethyl, or propyl. “Alkoxy” refers to alkyl groups attached via an oxygen, such as methoxy (—OCH₃), ethoxy (—O—CH₂—CH₃), or proplyoxy (e.g., —O—CH₂—CH₂—CH₃ or —O—CH(CH₃)₂), collectively referred to as C₁₋₃ alkoxy. “Halogen,” “halide,” and “halo” refers to —F, —Cl, —Br, and/or —I. “Alkylene” and “substituted alkylene” refer to divalent alkyl and divalent substituted alkyl, respectively, including, without limitation, ethylene (—CH₂—CH₂—).

Compound (1) may be used in the treatment of a patient, as described above for compounds 958 and 958ₐₘᵢ, e.g., in treating inflammation and inflammatory diseases, cardiovascular inflammation, vascular inflammation (e.g., having vascular endothelium inflammation or a disease having vascular endothelium inflammation as a symptom, such as pulmonary arterial hypertension, restenosis, essential hypertension, atherosclerosis, and stroke), a disease characterized by vascular inflammation, or a disease of innate and acquired immunity, such as autoimmune diseases, heart disease, lung disease, sepsis, cancer, and neurodegeneration, e.g., inflammation associated with, for example, heart disease, lung disease, sepsis, cancer, and neurodegeneration. The compound may be used to treat a viral infection, e.g. inflammation associated with a viral infection, such as SARS-CoV-2 or other SARS or MERS virus infections.

The therapeutically-effective amount of each compound for treatment of a disease as described herein may range from 1 pg per dose to 10 g per dose, including any amount there between, such as, without limitation, 1 ng, 1 μg, 1 mg, 10 mg, 100 mg, or 1 g per dose to a patient, for example from 10 ng/kg/day to 1 g/kg/day, or from 1 μg/kg/day to 100 mg/kg/day, or increments therebetween. The therapeutically-effective amount may range from 1 μg to 10 g, or from 1 ng to 100 mg/kg of compound 958, or an equivalent amount of another compound described herein, per day, or 20 μM±99%, 90%, 80%, 70%, 60%, 50%, 40%, 30%, 25%, 20%, 15%, 10%, 0.5%, 1%, or any increment therebetween in a patient's bodily fluid, e.g. blood, serum, plasma, etc. The therapeutic agent may be administered to a patient by any effective route, and, for example, as a single dose or bolus, at regular or irregular intervals, or continuously, in amounts and intervals as dictated by any clinical parameter of a patient.

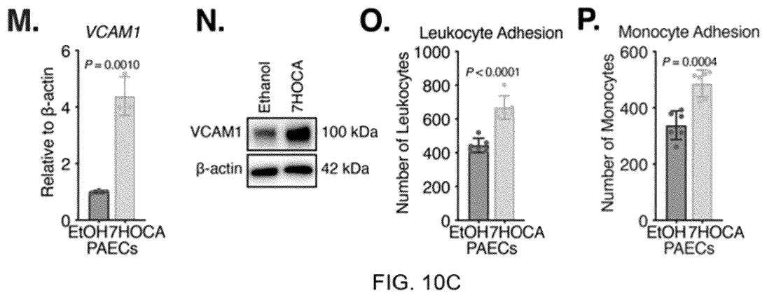

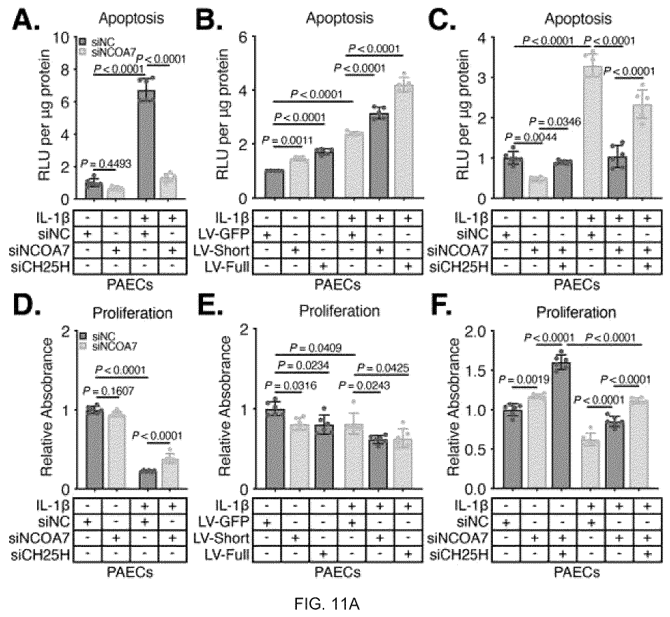

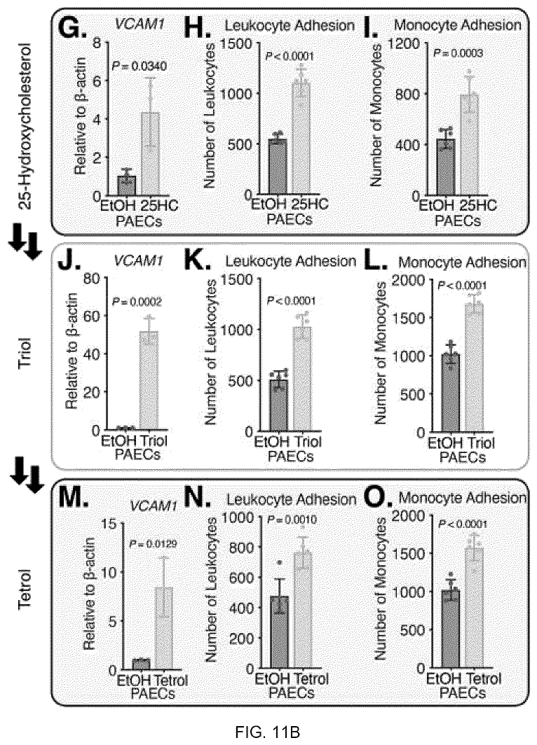

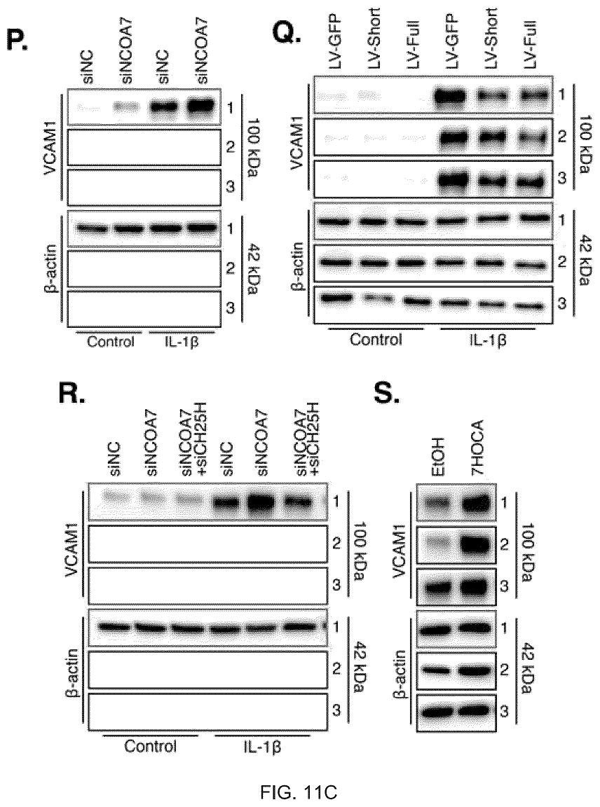

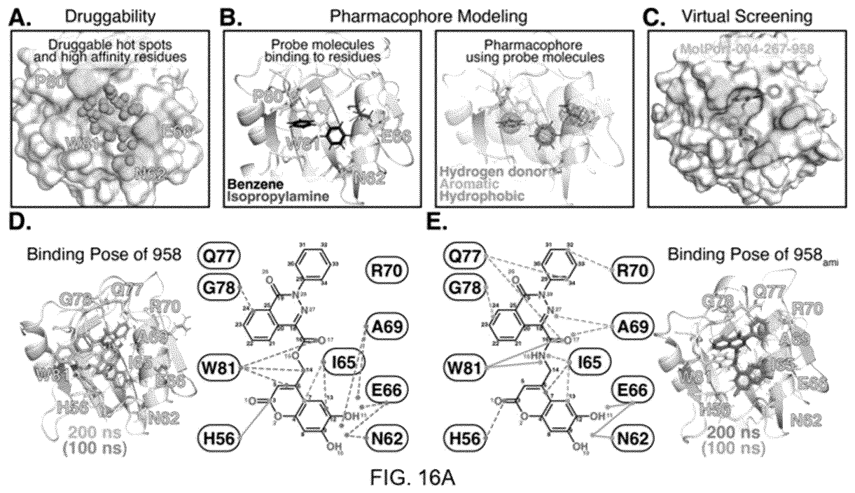

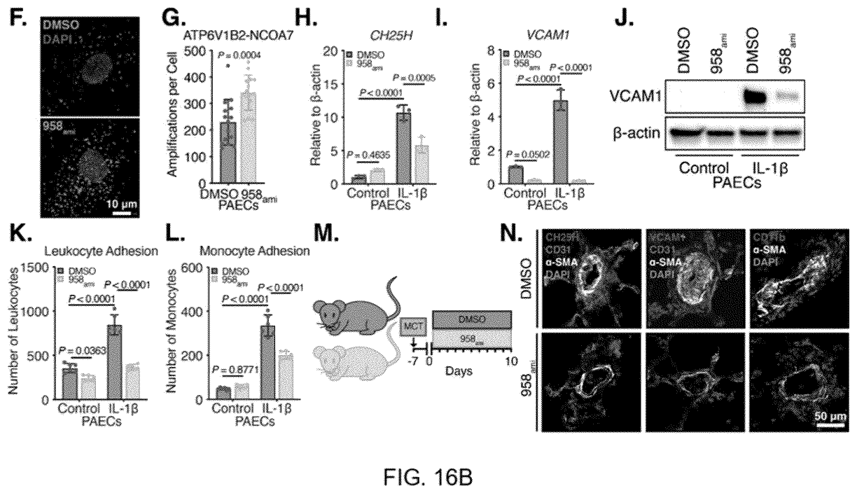

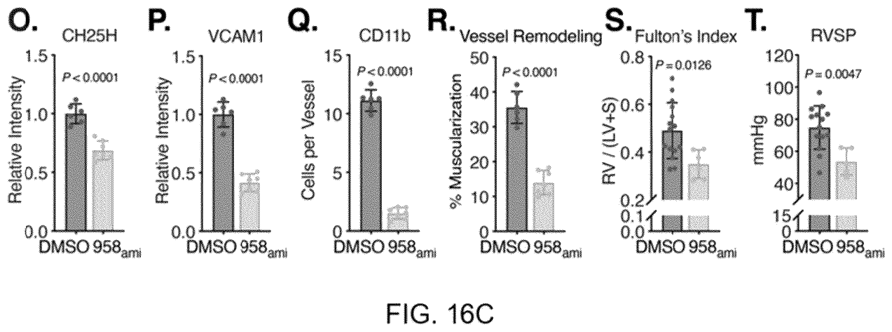

As demonstrated below, additional compounds are being evaluated as useful in treating inflammation and inflammatory diseases, cardiovascular inflammation, vascular inflammation (e.g., having vascular endothelium inflammation or a disease having vascular endothelium inflammation as a symptom, such as pulmonary arterial hypertension, restenosis, essential hypertension, atherosclerosis, and stroke), a disease characterized by vascular inflammation, or a disease of innate and acquired immunity, such as heart disease, lung disease, sepsis, cancer, and neurodegeneration, e.g., inflammation associated with, for example, heart disease, lung disease, sepsis, cancer, and neurodegeneration. The compound may be used to treat a viral infection, e.g. inflammation associated with a viral infection, such as SARS-CoV-2 or other SARS or MERS virus infections. The listed compounds in FIGS. 11, 16A-16E may be provided as pharmaceutically acceptable salts thereof, and may include equivalent derivative compounds, such as esters or prodrugs thereof.

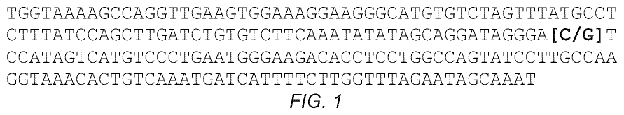

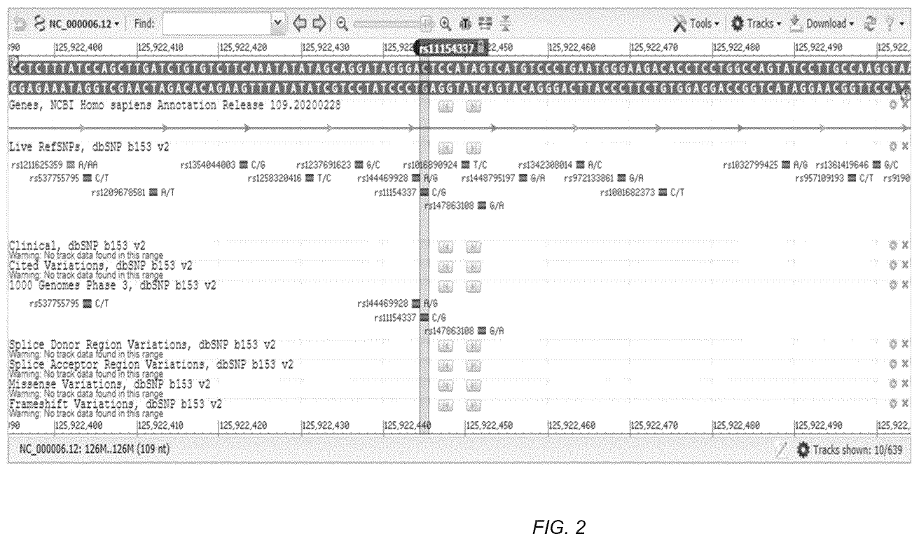

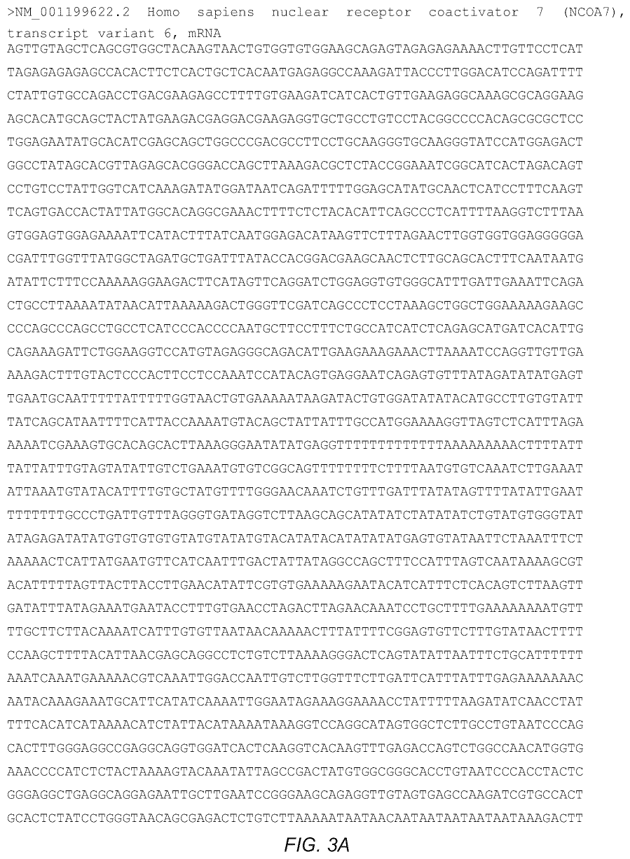

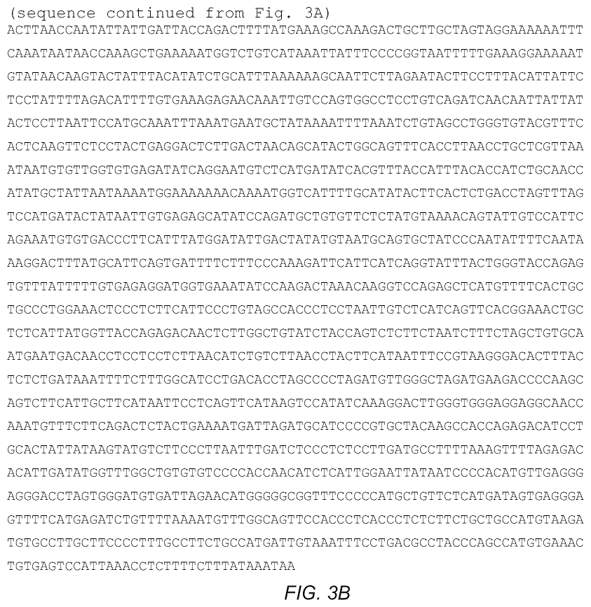

The single nucleotide polymorphism (SNP) rs11154337 is a polymorphism within the NCOA7 gene. FIGS. 1 and 2 show the sequence and location of rs11154337 in an intron of the NCOA7 gene (see, also, US Patent Publication No. 2021/0309998 A1, incorporated herein by reference in its entirety for its disclosure). FIGS. 3A and 3B provide an exemplary NCOA7 mRNA (cDNA) sequence.

NCOA7 is the protein product of the NCOA7 gene. An exemplary NCOA7 mRNA sequence (SEQ ID NO: 2) is provided in FIGS. 3A and 3B. The NCOA7 gene has a candidate SNP termed rs11154337 (SEQ ID NO: 1). An exemplary rs11154337 sequence is provided in FIGS. 1 and 2. rs11154337 is located in the promoter of an interferon-inducible isoform of NCOA7 (NCOA7short) that we first identified in an unpublished genome-wide association study (GWAS) of survival in human pulmonary arterial hypertension. Other mutations or polymorphisms located in the same intron as rs11154337, e.g., as shown in FIG. 2, or in linkage disequilibrium with rs11154337 may either be indicative of a high risk genotype and may functionally affect expression of NCOA7, and therefore may be, like rs11154337 or in combination therewith, useful in detecting persons especially susceptible to coronavirus infection, and correction of the risk polymorphism, e.g., by gene editing of a functional polymorphism that affects expression of NCOA7 may reduce infectivity of a coronavirus. SNP rs11154337 (SEQ ID NO: 1) exists at an intronic region where both the ReIA/p65 subunit of NF-kB and STAT1 are predicted to bind. From an antimicrobial defense perspective, this duality suggests a functional cooperation between two host defense pathways: (1) initial detection at the plasma membrane via Toll-like receptors and the NF-kB pathway and (2) potential endosomal pathogen escape that triggers an interferon-mediated response and STAT1/2 activation via Janus tyrosine kinases (JAK). As such, our molecular studies have defined NCOA7 as an upregulated factor in ECs in response to proinflammatory cytokines; moreover, both the inhibition of STAT1/2 signaling via the JAK inhibitor momelotinib and RNAi of ReIA/p65 abrogated the IL-1β-mediated upregulation of NCOA7. We have demonstrated that NCOA7 regulates immunoactivation of the endothelium and subsequent leukocyte adhesion and presumable infiltration. To do so, NCOA7 alters lysosomal acidification, a feature that has been independently found to affect entry of other enveloped viruses, such as influenza. Furthermore, utilizing an in vitro biochemical assay of nuclear protein binding to SNP rs11154337 and CRISPR-Cas9-edited, isogenic, inducible-pluripotent stem cell (iPSCs), we have found allele-specific binding to the NF-kB subunit ReIA/p65 that drives allele-specific expression of NCOA7.

A lysosome is a membrane-bound organelle found in many animal cells. Lysosomes are spherical vesicles that contain hydrolytic enzymes that can break down many kinds of biomolecules. A lysosome has a specific composition, of both its membrane proteins, and its lumenal proteins. The lumen's pH (˜4.5-5.0) is optimal for the enzymes involved in hydrolysis, analogous to the activity of the stomach. Besides degradation of polymers, the lysosome is involved in various cell processes, including secretion, plasma membrane repair, apoptosis, cell signaling, and energy metabolism. Lysosomes act as the waste disposal system of the cell by digesting in used materials in the cytoplasm, from both inside and outside the cell. Material from outside the cell is taken up through endocytosis, while material from the inside of the cell is digested through autophagy. The size of lysosomes varies from 0.1 μm to 1.2 μm. Lysosomes have a pH ranging from ˜4.5-5.0, accordingly, the interior of the lysosomes is acidic compared to the slightly basic cytosol (pH 7.2). The lysosome maintains its pH differential by pumping in protons (H⁺ ions) from the cytosol across the membrane via proton pumps and chloride ion channels. Vacuolar-ATPases are responsible for transport of protons, while the counter transport of chloride ions is performed by ClC-7 Cl⁻/H⁺ antiporter.

Our work establishes a paradigm that links lysosomal biology and oxysterol and bile acid metabolism with endothelial inflammation, offering broad implications on precision medicine approaches in pulmonary arterial hypertension. We also found evidence of genetic association of this NCOA7 SNP with essential hypertension, atherosclerosis, and stroke, making this target relevant for these other vascular diseases. In pilot data, we have found an association of these same oxysterols and bile acids in sepsis, suggesting that targeting NCOA7 would be beneficial in this disease of critical illness as well as in diseases of acquired and innate immunity in general.

In this process, we used a computational prediction approach to identify specific small molecules that may activate or inhibit NCOA7 based on a high predicted likelihood of binding to the enzymatic pocket of the Tre2/Bub2/Cdc16 (TBC), lysin motif (LysM), domain catalytic (TLDc) domain of NCOA7. One of the top hits, compound 958 (e.g., 6,7-dihydroxy-2-oxo-2H-chromen-4-yl)methyl 4-oxo-3-phenyl3,4-dihydrophthalazine-1-carboxylate, was found to carry predicted activity of altering lysosomal pH and down-regulating cholesterol metabolism as well as inflammatory activation in endothelial cells. They may also carry such therapeutic activity in other non-vascular diseases of acquired and innate immunity. Finally, they may serve as a new therapy for COVID-19, supported by our pilot data showing that this compound can inhibit entry of SARS-CoV-2 pseudotype virus into human cells.

In aspects and embodiments:

1. Invention is based on novel and unpublished mechanistic data describing the role of NCOA7 in controlling lysosomal activity, sterol homeostasis, and inflammation.

2. Notably, the invention targets a novel pathway and set of targets not previously attempted in cardiopulmonary vascular disease, sepsis, or COVID-19.

3. Importantly, relevance of NCOA7 and the target pathway to human pulmonary arterial hypertension, essential hypertension, stroke, and atherosclerosis is based on population-level human genetic and metabolomic data.

4. The molecule (6,7-dihydroxy-2-oxo-2H-chromen-4-yl)methyl 4-oxo-3-phenyl3,4-dihydrophthalazine-1-carboxylate has no known protein target and has not been studied in the context of any human disease or inflammation in general.

5. The computational process by which these small molecule predictions have been made has never been applied to NCOA7 or TLDc domains in the past.

In a further embodiment, a method of treating a patient having a C at SNP rs11154337 and having inflammation and/or an inflammatory disease, such as, cardiovascular inflammation, vascular inflammation (e.g., having vascular endothelium inflammation or a disease having vascular endothelium inflammation as a symptom, such as pulmonary arterial hypertension, restenosis, essential hypertension, atherosclerosis, and stroke), a disease characterized by vascular inflammation, or a disease of innate and acquired immunity, such as autoimmune diseases, heart disease, lung disease, sepsis, cancer, and neurodegeneration, e.g., inflammation associated with, for example, heart disease, lung disease, sepsis, cancer, and neurodegeneration is provided. The method comprises using gene editing, such as, for example and without limitation, a CRISPR/Cas9- or TALEN-based gene editing method to change one or more C at SNP rs11154337 to a G (see, e.g., Abdelnour SA, Xie L, Hassanin A A, Zuo E, Lu Y. The Potential of CRISPR/Cas9 Gene Editing as a Treatment Strategy for Inherited Diseases. Front Cell Dev Biol. 2021 Dec. 15; 9:699597 and Chiao-Lin Chen, Jonathan Rodiger, Verena Chung, Raghuvir Viswanatha, Stephanie E Mohr, Yanhui Hu, Norbert Perrimon, SNP-CRISPR: A Web Tool for SNP-Specific Genome Editing, G3 Genes/Genomes/Genetics, Volume 10, Issue 2, 1 Feb. 2020, Pages 489-494, for description of the CRISPR/CAS9 technology and tools useful in implementation of such technology and tools, and also commercial services for gene editing such as using technology and expertise provided by CRISPR Therapeutics of Cambridge, MA).

### EXAMPLES

By harnessing large-scale and multi-dimensional metabolomic and genomic data with mechanistic experimentation in vitro and in vivo, NCOA7 was found to control lysosomal activity and endothelial sterol homeostasis to act as a homeostatic brake, tempering oxysterol- and 7HOCA-induced inflammation, endothelial dysfunction, and PAH. Furthermore, when the G allele of NCOA7 intronic SNP rs11154337 is present, NCOA7 is increased, thus reducing inflammation in PAH and offering mechanistic proof underlying the genetic association of SNP rs11154337 to PAH disease severity, the metabolomic association of the oxysterol and bile acid signature to PAH severity, and the genetic association of SNP rs11154337 to 7HOCA plasma levels. Identification of active pharmaceutical ingredients, e.g. drugs, capable of reducing inflammation, e.g., endothelial inflammation provides useful therapeutics for treatment of a variety of diseases relating to inflammation, including cardiovascular inflammation, vascular inflammation (e.g., having vascular endothelium inflammation or a disease having vascular endothelium inflammation as a symptom, such as pulmonary hypertension, restenosis, essential hypertension, atherosclerosis, and stroke), a disease characterized by vascular inflammation, or a disease of innate and acquired immunity. The following examples are illustrative and proof of concept of the invention as claimed.

### Example 1

Vascular inflammation critically regulates endothelial cell (EC) pathophenotypes, yet causative mechanisms remain incompletely defined, particularly in pulmonary arterial hypertension (PAH). Immune dysregulation and metabolic reprogramming are recognized tenets of PAH pathogenesis, but a unifying theory connecting the two has not been established. In human pulmonary arterial ECs, induction of the nuclear receptor coactivator 7 (NCOA7), a gene previously identified as upregulated in PAH, tempered the generation of proinflammatory sterols by bolstering lysosomal acidification and constraining EC immunoactivation. Conversely, reduced NCOA7 promoted lysosomal dysfunction, generating proinflammatory sterol and bile acids that drive EC phenotypes consistent with PAH. In vivo, mice deficient for Ncoa7 or treated with exogenous 7α-hydroxy-3-oxo-4-cholestenoic acid (7HOCA)—a representative metabolite from the NCOA7-dependent sterol signature—demonstrated worsened PAH. Emphasizing the clinical importance of this mechanism in controlling disease severity, an unbiased, metabolome-wide association study from the multicenter PAH Biobank cohort (N=2,796) identified a plasma signature inclusive of the same NCOA7-dependent sterols and bile acids associated with PAH mortality (adjusted P<1.1×10⁻⁶). Indicating a potentially widespread genetic predisposition to NCOA7 deficiency, the common variant intronic SNP rs11154337 in NCOA7 was found to control NCOA7 levels, lysosome activity, sterol and bile acid production, and EC immunoactivation in isogenic, SNP-edited, iPSC-derived ECs. Correspondingly, SNP rs11154337 was associated with PAH severity as noted by six-minute walk distance (P=0.0130, β=58.15, 95% Cl [14.45-119.36]) and mortality (P=0.0250; hazard ratio=0.44, 95% Cl [0.21-0.90]) in an independent, single-center PAH cohort (N=93). In a second validation, multi-center PAH cohort (N=630), SNP rs11154337 was further associated to mortality (P=0.0002; hazard ratio=0.49, 95% Cl [0.34-0.71]). Lastly, utilizing computational modeling and simulations of NCOA7 functional domain, we predicted and synthesized a novel activator of NCOA7 that prevents endothelial immunoactivation and reverses indices of PAH in the monocrotaline rat. In sum, this work establishes a genetic and metabolic paradigm that links lysosomal biology and sterol and bile acid processes with EC inflammation and proposes a novel therapeutic. This paradigm carries broad implications not only on molecular diagnostic and therapeutic treatment of PAH but also in other vascular disorders dependent upon acquired and innate immune regulation.

Vascular inflammation critically regulates endothelial cell (EC) behaviors across vascular diseases such as atherosclerosis, hypertension, stroke, sepsis, and many others. Within the lung, inflammation of the endothelium is a prominent feature of acute lung injury, pathogen-mediated processes such as SARS-CoV-2 infection, and pulmonary arterial hypertension (PAH)—a deadly and enigmatic vascular disease characterized by complex vessel remodeling and poorly defined molecular origins. Yet, the exact causative role for inflammation in PAH has been debated with the possibility that inflammation exerts more obvious control over PAH severity rather than overall risk.

Molecular homeostatic accelerators and brakes regulating inflammation are critical in the maintenance of cellular function. However, the specific levers that control inflammation to cause EC dysfunction, such as in PAH, are incompletely described. Lysosomal activity is increasingly appreciated as a principal regulator of inflammation, and dysfunctional lysosomal activity has been observed in PAH. Central to the maintenance of lysosomal enzyme function is proper acidification of the luminal space, which is mediated by the vacuolar H⁺ ATPase (V-ATPase) family, and loss of this hydrolytic capacity results in a disease class known as lysosomal storage disorders (LSDs) that have reported pulmonary vascular phenotypes. The nuclear receptor co-activator 7 (NCOA7) directly binds and modulates V-ATPase activity to control endolysosomal function, which has documented function in controlling bacterial and viral pathogen entry, renal tubular acidification, and neuronal function. NCOA7 is upregulated in human ECs by proinflammatory stimuli and in PAH lung tissue, but any causative mechanism connecting NCOA7 to cardiopulmonary vascular disease has not been defined.

Downstream of acidification, lysosomes carry pH-sensitive, hydrolytic enzymes responsible for the breakdown of cellular waste and macromolecular trafficking. The lysosomal-mediated breakdown of cellular waste is connected to autophagy—a process that may be relevant in PAH. Notably, loss of lysosomal hydrolase activity leads to the accumulation of oxysterols and bile acids, which are bioactive molecules upregulated in the plasma and lungs of PAH patients. Oxysterols and bile acids influence cholesterol biosynthesis and cell membrane properties, driving critical cellular defenses in adaptive and innate immunity. At the level of the endothelium, these molecules are capable of endothelial immunoactivation to contribute to peripheral vascular diseases like atherosclerosis and hypertension. It also recently has been found in an unbiased plasma metabolomic analysis of 2,796 PAH patients that a metabolome-wide association (adjusted P<1.1×10⁻⁶) between a signature of glucuronidated oxysterols and downstream bile acids with both indices of PAH disease severity and mortality. As such, given the inherent connection of NCOA7 to both lysosomal biology and a metabolome-wide association of lysosome-derived oxysterol and bile acid levels with PAH mortality, we sought to determine if NCOA7 controls oxysterol and bile acid metabolism, inflammatory pulmonary EC pathophenotypes, and the development of PAH. Elucidation of these connections would provide a mechanistic explanation underlying the association of glucuronidated oxysterols and bile acids with PAH mortality and severity.

### Methods

RNA extraction and quantitative polymerase chain reaction: Cells were lysed in QIAzol Lysis Reagent (Qiagen; 79306), and RNA was extracted using the Rnaeasy Kit (Qiagen; 74004). Complementary DNA was synthesized using the High-Capacity cDNA Reverse Transcription Kit (ThermoFisher; 4368813). Quantitative real-time PCR (RT-qPCR) was performed on an Applied Biosystems QuantStudio 6 Flex Real-Time PCR instrument.

Target gene expression was normalized to a housekeeping gene (i.e., ACTB) and fold change was calculated using the 2^{ΔΔCt} method. TaqMan™ Universal PCR Master Mix (ThermoFisher; 4305719) was used with TaqMan primers (Table 1). PowerUp™ SYBR^{T}M Green Master Mix (ThermoFisher; A25742) was used with custom designed primers (Table 2).

TABLE 1
TaqMan primers.

| Gene Target | Species | TaqMan Assay ID |
|---|---|---|
| ACTB | Human | Hs99999903_m1 |
| ATP6V1B2 | Human | Hs00156037_m1 |
| CH25H | Human | Hs02379634_s1 |
| LDLR | Human | Hs01092524_m1 |
| NCOA7 | Human | Hs00906291_m1 |
| VCAM1 | Human | Hs01003372_m1 |

TABLE 2
Custom primers for gene expression.

| Gene | | Primer Sequences | SEQ ID |
|---|---|---|---|
| Target | Species | 5′ to 3′ | NO: |
| ACTB | Human | Forward: | 3 |
| | | CATGTACGTTGCTATCCAGGC | |
| | | Reverse: | 4 |
| | | CTCCTTAATGTCACGCACGAT | |
| NCOA7ₛₕₒᵣₜ | Human | Forward: | 5 |
| | | GCCACACTTCTCACTGCTCA | |
| | | Reverse: | 6 |
| | | TAGGACAGGCAGCACCTCTT | |
| NCOA7_{full} | Human | Forward: | 7 |
| | | CCCGCTGCAAGATGGAAGG | |
| | | Reverse: | 8 |
| | | CGAGTAGCCATCCTGCAACT | |
| Actb | Mouse | Forward: | 9 |
| | | ACCTTCTACAATGAGCTGCG | |
| | | Reverse: | 10 |
| | | CTGGATGGCTACGTACATGG | |
| Ncoa7ₛₕₒᵣₜ | Mouse | Forward: | 11 |
| | | GCAGGCAACCAGAGAAAGAC | |
| | | Reverse: | 12 |
| | | CGTTTTGCCTCCTCAACTGT | |
| Ncoa7_{full} | Mouse | Forward: | 13 |
| | | ATGGAAAGGGTGTGGTTGGG | |
| | | Reverse: | 14 |
| | | CTCCAGGCCTGTACAGAGGA | |

Immunofluorescent staining of lung tissue: OCT-embedded lung tissue was sliced using a cryostat at thickness of 5 to 7 microns and were subsequently mounted onto gelatin-coated histological slides. Sections were rehydrated with PBS for five minutes, fixed in 2% PEA for 30 minutes, permeabilized in 0.1% Triton X-100 for 15 minutes, and blocked in 5% donkey serum and 2% BSA in PBS for one hour at room temperature. Primary antibodies (Table 3) were diluted in 2% BSA and incubated overnight at 400. AlexaFluor conjugated secondary antibodies were used (ThermoFisher) at a dilution of 1:1000 in 2% BSA for one hour at room temperature. Sections were then counterstained with Hoescht for one minute at room temperature and then mounted. Small pulmonary vessels (30 to 100 microns in diameter) not associated with a bronchial airway were selected for imaging.

TABLE 3
Antibodies for protein detection.

| | | | |
|---|---|---|---|
| Target | Species | Concentration | Vendor |

Flow Cytometry

| | | | |
|---|---|---|---|
| CD34-FITC | Mouse | 1:200 | BD Biosciences; 555821 |
| CD144-APC | Mouse | 1:200 | BioLegend; 348508 |
| CD309-PE | Mouse | 1:200 | BD Biosciences; 560872 |

Immunoblot

| | | | |
|---|---|---|---|
| Myc | Rabbit | 1:1000 | Cell Signaling |
| | | | Technologies; 2278S |

Immunofluorescence

| | | | |
|---|---|---|---|
| αSMA | Goat | 1:100 | Sigma-Aldrich; A5228 |
| ATP6V1B2 | Rabbit | 1:100 | abcam; ab73404 |
| CD31/PECAM-1 | Goat | 1:100 | R&D Systems; AF3628 |
| CH25H | Rabbit | 1:100 | abcam; ab214295 |
| NCOA7 | Rabbit | 1:100 | abcam; ab224481 |
| VCAM1 | Rabbit | 1:100 | abcam; ab134047 |

Immunoprecipitation

| | | | |
|---|---|---|---|
| IgG | Rabbit | 1 μg/μL | abcam; ab171870 |
| NF-κB p65 | Rabbit | 5 μg/μL | abcam; ab19870 |

Proximity Ligation Assay

| | | | |
|---|---|---|---|
| ATP6V1B2 | Rabbit | 1:50 | abcam; ab73404 |
| NCOA7 | Mouse | 1:50 | Santa Cruz Biotechnology; |
| | | | sc-393427 |

Single-cell transcriptomics: Single cell RNA sequencing was performed on lungs of healthy controls and idiopathic PAH patients. Expression matrices were derived using CellRanger. Subsequent batch correction, scaling, and normalization were all performed using SCTransform in Seurat v3. Cell types were determined with SingleR using the Blueprint ENCODE reference. Cells were identified as positively expressing NCOA7if the transformed expression value was greater than 0. Cells expressing NCOA7 were identified as having a transformed expression value greater than 0.2.

Generation of iPSCs and CRISPR-Cas9 gene-editing: To generate isogenic lines, we introduced the G allele for SNP rs11154337 into control iPSCs with pSpCas9(BB)-2A-GFP (PX458; Addgene; 48138) as the vector for genome editing. For the site-specific CRISPR-Cas9 and guide RNA sequence construction, two reverse complementary guide oligos were annealed and ligated to the linearized PX458 vector (Table 4). The single-stranded oligodeoxynucleotide (ssODN) template with mutation/correction sites was designed as previously described (F. A. Ran et al., Genome engineering using the CRISPR-Cas9 system. Nat Protoc 8, 2281-2308 (2013)). Control iPSCs were dispersed as single cells the day before transfection at 40 to 50% confluency. The next day, CRISPR-Cas9 and ssODN templates were transfected into iPSCs using GeneJammer reagent according to the manufacturer's protocol (Agilent Technologies 204132). After 36 hours, GFP positive cells were sorted using FACS and were seeded at a density of 2,000 cells per well in a 6-well plate. After expansion for one-week, single cell-derived colonies were observed and picked for genotype characterization and subsequent expansion (table S4). DNA was extracted from collected cells using QuickExtract solution (Epicenter). PrimeSTAR® GXL DNA Polymerase (Clontech) was used for PCR amplification and genotyping of the edited region.

TABLE 4
Oligonucleotides for genome editing.

| Oligo- | | | SEQ |
|---|---|---|---|
| nucleo- | | | ID |
| tides | Species | Sequence 5′ to 3′ | NO: |
| NCOA7 | Human | Forward: | 15 |
| gRNAs | | CACCGTTCAAATATATAGCAGGATA | |
| | | Reverse: | 16 |
| | | AAACTATCCTGCTATATATTTGAAC | |
| NCOA7 | Human | CTAAACCAAGAAAATGATCATTTGA | 17 |
| ssODN | | CAGTGTTTACCTTGGCAAGGATACT | |
| | | GGCCAGGAGGTGTCTTCCCATTAGG | |
| | | GACATGACTATGGACTCACTATCCT | |
| | | GCTATATATTTGAAGACACAGATCA | |
| | | A | |
| NCOA7 | Human | Forward: | 18 |
| Sequencing | | CTTCATGGCCTCCTTTGGTA | |
| Primer | | Reverse: | 19 |
| Set #1 | | AAGGATACTGGCCAGGAGGT | |
| NCOA7 | Human | Forward: | 20 |
| Sequencing | | CCAGGTTGAAGTGGAAAGGA | |
| Primer | | Reverse: | 21 |
| Set #2 | | GGACAGGCCTCACCTGATTA | |

Differentiation of iPSCs into endothelial cells: The creation of iPSC-ECs was done using a chemical differentiation protocol with three major steps: mesoderm induction, endothelial specification, and iPSC-EC purification. Briefly, mesoderm induction was done through Wnt signaling activation using the glycogen synthase kinase-3β inhibitor CHIR99021 (Selleckchem; S2924) in RPMI medium (Life Technologies; 11875-093) with B-27 minus insulin (Life Technologies; A18956-01) supplementation. The use of insulin-free B27 is believed to improve differentiation efficiency, as insulin negatively affects mesoderm induction. Next, endothelial lineage specification was done using supplemented growth factors like vascular endothelial growth factor (VEGF; 50 ng/mL; Gemini; 300196P) and fibroblast growth factor (FGF; 25 ng/mL; Gemini; 300113P) in EGM™_2 Endothelial Cell Growth Medium-2 BulletKit™. To increase yield, the transforming growth factor β (TGFβ) inhibitor SB431542 (10 μM; Selleckchem; S1067) was also added, as it promotes EC generation and inhibits smooth muscle cell differentiation from endothelial progenitors. Lastly, iPSC-EC purification was done using magnetic-activated cell sorting (MACS) against the mature EC surface marker vascular endothelial (VE)-cadherin, also known as CD144. Mature iPSC-ECs were labeled with magnetic CD144 MicroBeads (Miltenyi Biotech; 130-097-857), captured by a column in a magnetic field, and then separated from the unlabeled cells. Purified iPSC-Ecs were maintained in EGM™_2 Endothelial Cell Growth Medium-2 BulletKit™.

Characterization of iPSC-Ecs by flow cytometry: Cells before and after CD144⁺ purification were analyzed using flow cytometry against endothelial surface markers. Specifically, expression of the mature endothelial progenitor marker CD34 (FITC Anti-CD34 Clone 581; BD Pharmingen™; 555821), the mature endothelial marker VE-cadherin/CD144 (APC Anti-CD144 Clone Bv9; BioLegend; 348508), and the vascular endothelial growth factor receptor 2 (VEGFR2, or CD309; PE Anti-CD309 Clone 89106; BD Pharmingen™; 560872) were assessed (Table 3). Samples were run on the BD LSRFortesssa™ Flow Cytometer (BD Biosciences) at the Unified Flow Core at the University of Pittsburgh.

Characterization of iPSC-ECs by immunofluorescent staining: After CD144⁺ purification, iPSC-ECs were further characterized by immunofluorescent staining of cell surface markers. Briefly, cells were fixed with 4% paraformaldehyde (PFA) for 15 minutes at room temperature, and then blocked in 5% bovine serum albumin (BSA) for one hour at room temperature. Cells were stained with Anti-VE-cadherin/CD144 antibody (1:100; abcam; ab33168) or Anti-CD31 (also known as platelet and endothelial cell adhesion molecule-1; PECAM-1; 1:100; abcam; ab24590) overnight at 4° C. (Table 3). After rinsing with PBS, cells were incubated with appropriate secondary antibodies (1:1000) in 5% BSA for one hour at room temperature. Cells were rinsed with PBS and mounted with ProLong™ Gold Antifade Mountant with DAPI (ThermoFisher; P36935). Images were acquired on a Nikon A1 Confocal Microscope.

Characterization of iPSC-ECs by in vitro tube formation: To confirm an endothelial phenotype, a capillary-like tube formation assay was performed using the in vitro Angiogenesis Assay (R&D Systems; 3470-096-K) (DeCicco-Skinner et al., 2014). A basement membrane extract with reduced growth factors was plated onto a 96-well plate and allowed to solidify for 30 minutes at 37° C. iPSC-ECs were then plated (20,000 cells per well) into the well with cell-type specific media deficient for growth factors and serum. After six hours, capillary-like structures were imaged with the EVOS™ XL Core Imaging System (ThermoFisher) at 10× magnification.

Transfection of cells for RNA silencing: Human PAECs were transfected at approximately 70 to 80% confluency using negative control (siNC) or target gene (siGene) silencing RNA (siRNA) at 20 nM (Table 5). Lipofectamine® 2000 (ThermoFisher; 11668019) was mixed with siRNA per manufacturer's protocol. Lipofectamine®:siRNA mixture was incubated with human PAECs in Opti-MEM™ reduced serum media for 4 to 6 hours (ThermoFisher; 31985062). After incubation, transfection media was removed and replaced with full serum, cell-specific growth media. Experiments were performed 48 hours post-transfection.

TABLE 5
Silencing RNA species.

| siRNA Gene Target | Species | Silencer ™ Assay ID |
|---|---|---|
| CH25H | Human | 289454 |
| NCOA7 | Human | 242114 |
| Negative Control No. 1 | Human | AM4611 |

Construction of lentiviral plasmids and particles: The cDNA sequences encoding full-length NCOA7 (mRNA transcript variant 1, NM_181782.5) and short-length NCOA7 (mRNA transcript 6, NM_001199622.2) were amplified by PCR with Hindlll and Nhel linkers. The PCR products were directly cloned downstream of Myc-tagged green fluorescent protein (mGFP) open reading frame in the pmGFP-ADAR1-p110 vector (Addgene; 117928). The cDNA sequences encoding mGFP fused full- and short-length NCOA7 were further subcloned into the pCDH-CMV-MCS-EF1α-Puro lentiviral expression vector (Systems Biosciences; CD510B-1). Cloned plasmids were verified by DNA sequencing at the Genomics Research Core at the University of Pittsburgh.

HEK293T cells were maintained in Dulbecco's Modified Eagle Medium (DMEM) with 10% FBS. HEK293T cells were transfected using Lipofectamine® 2000 (ThermoFisher; 11668019) with lentiviral plasmids containing the target gene (or an empty vector for control virus) and packaging plasmids from the ViraPower™ Lentiviral Packaging Mix (ThermoFisher; K497500). Viral particles were harvested 48 hours after transfection, pelleted, and then filtered.

Transduction of cells for lentiviral vector delivery: Human PAECs were transduced by direct application of media with polybrene (8 μg/mL) containing viral particles with an empty, control vector expressing GFP or with the target gene. Transduction efficiency was assessed via GFP expression and direct measures of transcript and protein expression. Experiments were performed 72 hours after transduction.

Protein extraction and immunoblotting: Cells were rinsed two times with PBS before collection in RIPA buffer containing protease and phosphatase inhibitors. Protein concentration was determined using the Pierce™ BCA Protein Assay Kit (ThermoFisher; 23225). Protein lysates were separated using 4-15% Mini-PROTEAN® TGX™ Precast Protein Gels (Bio-Rad Laboratories; 4561086) and subsequently transferred onto a PVDF membrane. Membranes were blocked with 5% BSA in Tris-buffered saline with 0.1% Tween 20 (TBST) for one hour at room temperature. Primary antibodies were subsequently added and incubated at 4° C. overnight (Table 3). The next day blots were washed three times for 10 minutes each with TBST. Blots were then incubated with the appropriate secondary antibody coupled to HRP for one hour at room temperature. After another round of TBST washing, blots were visualized using Pierce ECL reagents and images were captured using the BioRad ChemiDoc XRS+.

Chromatin immunoprecipitation and quantitative polymerase chain reaction: The MAGnify™ Chromatin Immunoprecipitation System (Invitrogen; 49-2024) per the manufacturer's protocol. Briefly, 1×10⁶ human PAECs or iPSC-derived ECs were utilized for each ChIP reaction. Dynabeads® were coupled to either rabbit IgG antibody (1 μg/μL) or rabbit NF-κB p65 antibody (5 μg/μL) for one hour at 4° C. (Table 3). 1×10⁶ cells were trypsinized, pelleted, and resuspended in 500 μL per reaction. Each reaction was crosslinked with 1% methanol-free formaldehyde for 10 minutes at room temperature. The crosslinking reaction was inhibited with 1.25 M glycine for five minutes at room temperature. From this point forward, the reaction was kept at 4° C. for all steps. Samples were pelleted and washed three times in cold PBS. Each reaction was then resuspended in 50 μL lysis buffer with protease inhibitor before proceeding to chromatin shearing. The Biorupter® UCD-200 was used to shear cells using a protocol of 20 seconds ON and 40 seconds OFF for six cycles. Samples were pelleted and supernatant containing the chromatin products was collected and confirmed via DNA gel to have appropriate fragmentation. Antibody-bound Dynabeads® were incubated with chromatin for two hours at 4° C. while rotating end-over-end. Samples were then washed with a series of immunoprecipitation buffers before crosslinking reversal with proteinase K. The DNA was then purified before proceeding to quantitative PCR. Primers utilized for ChIP-qPCR are listed in Table 6.

TABLE 6
Custom primers for ChIP qPCR.

| | | | SEQ |
|---|---|---|---|
| | | Primer Sequences | ID |
| DNA Target | Species | 5′ to 3′ | NO: |
| Non-canonical | Human | Forward: | 22 |
| Promoter | | AAAGCTAGGTTCACTGGAGGG | |
| (i.e., | | Reverse: | 23 |
| NCOA7ₛₕₒᵣₜ) | | GGCATCGCTGTGAGACTGTAA | |
| Canonical | Human | Forward: | 24 |
| Promoter | | TGGGTGGTATGCCTAGTGAA | |
| (i.e., | | Reverse: | 25 |
| NCOA7_{full}) | | TTAAGGCTGGGCTGTAAGGT | |
| SNP | Human | Forward: | 26 |
| rs11154337 | | ACCTCCTGGCCAGTATCCTT | |
| Region | | Reverse: | 27 |
| | | ACTCACATAGTGCCCCTCCT | |

Chromatin conformation capture (3C) assay: A 3C assay was performed. Briefly, 1×10⁷ human PAECs were crosslinked with 1% formaldehyde at room temperature for 10 minutes. Nuclei were isolated and genomic DNA was digested with 400 U BspHI overnight at 37° C., 950 rpm. Per prediction by putative BspHI cutting sites along the genome, the restriction enzyme digestion generates a 1,666 bp genomic DNA fragment containing SNP rs11154337, and a 9,269 bp DNA fragment containing the promoter of NCOA7. Digested DNA was diluted and ligated with or without 4000U T4 DNA ligase for four hours in a 16° C. water bath, and then crosslinked DNA was reversed with 100 μg proteinase K at 65° C. overnight. DNA was then isolated and purified. PCR was performed with primers chosen to target the potential ligated fusion sequences of the DNA fragment containing SNP rs11154337 and fragment containing the promoter of NCOA7, and close to the BspHI site: (1) 50 bp from the BspHI site at the 3′ end of NCOA7 promoter fragment, 5′-TTT GGG CAA TGT TAC AGC AA-3′ (forward primer, (SEQ ID NO: 28)) and (2) 57 bp from BspHI site at the 5′ end of the SNP rs11154337 fragment, 5′-GAA ATG CCA GGG ATT CCT TA-3′ (reverse primer, (SEQ ID NO: 29)). The amplified product resulted in a 107 bp fragment to confirm the existence of the fusion sequence. PCR products were separated by gel electrophoresis and analysed by DNA sequencing.

Transcriptomic analysis of human PAECs and data availability: Microarray data were obtained using the Affymetrix Clariom D Human Array at the Genomics Research Core at the University of Pittsburgh. The microarray chip was performed on four groups in triplicate for a total of 12 samples. PAECs were subjected to either knockdown control or of the gene NCOA7. Additionally, groups were then either left in control conditions or further challenged with the proinflammatory cytokine IL-1p for 24 hours. Raw data were processed using Bioconductor packages in the language R to produce a list of differentially expressed genes that were selected using a Benjamini-Hochberg corrected p-value less than 0.05 in order to minimize the false discovery rate (FDR). The following add-on packages were utilized: oligo, limma, affycore tools, gplots, pd.clariom.d.human for the analysis of this microarray data. Very broadly, the flow of this program serves to take raw microarray data in .CEL format into a list of differentially expressed genes. The workflow included loading the raw data into R, filtering and normalizing the raw data, plotting to demonstrate uniformity among the samples, adding annotations, setting up a comparison matrix to run statistical analyses among the groups, generating a list of differentially expressed genes, filtering said list, constructing a heat map to depict generalized trends, and to overlap the produced list of genes with our PH Network. Gene-set enrichment analyses (GSEA) of identified differentially expressed genes were then created using Gene Ontology, REACTOME, KEGG, and BioCarta.

Proximity ligation assay: Direct interaction of NCOA7 with the V-ATPase subunit ATP6V1B2 was assessed using the Duolink® Proximity Ligation Assay (Millipore Sigma; DU092102). Human PAECs were plated in a Nunc™ Lab-Tek™ II Chamber Slide™ System (20,000 per well; ThermoFisher; 154453) and then fixed with 4% paraformaldehyde for 15 minutes. After permeabilization and blocking per the manufacturer's protocol, wells were incubated with either mouse anti-NCOA7 antibody (Santa Cruz Biotechnology; sc-393427), rabbit anti-ATP6V1B2 antibody (abcam; ab73404), both antibodies, or neither antibodies overnight at 4° C. (Table 3). Cells were then incubated with the PLUS and MINUS probes for one hour, ligated for 30 minutes, and amplified for 100 minutes at 37° C. Slides were then mounted with ProLong™ Gold Antifade Mountant with DAPI (ThermoFisher; P36935). Images were acquired on a Nikon A1 Confocal Microscope at the Center for Biologic Imaging at the University of Pittsburgh.

Transmission electron microscopy: Human PAECs grown on tissue culture plasticware were fixed in 2.5% glutaraldehyde in 100 mM PBS (8 gram/L NaCl, 0.2 gram/L KCl, 1.15 gram/L Na₂HPO₄·7H₂O, 0.2 gram/L KH₂PO₄, pH 7.4) overnight at 4° C. Monolayers were washed in PBS three times and then post-fixed in aqueous 1% osmium tetroxide, 1% Fe₆CN₃ for one hour. Cells were washed three times in PBS and then dehydrated through a 30-100% ethanol series with several changes of Poly/Bed® 812 embedding resin (Polysciences). Cultures were embedded by inverting Poly/Bed® 812-filled BEEM® capsules on top of the cells. Blocks were cured overnight at 37° C., and then cured for two days at 65° C. Monolayers were pulled off the coverslips and re-embedded for cross sectioning. Ultrathin cross sections (60 nm) of the cells were obtained on a Riechert/Leica UltraCut E ultramicrotome, post-stained in 4% uranyl acetate for 10 minutes and then 1% lead citrate for seven minutes. Sections were viewed on a JEOL JEM-1400Flash transmission electron microscope at 80 kV. Images were taken using a bottom mount AMT digital camera. Acquired micrographs were analyzed manually in a blinded manner. Lysosomal area was quantified using Fiji.

Assessment of lysosomal hydrolase activity: Lysosomal activity and function were assessed using measures of enzyme activity. Human PAECs were plated on glass coverslips and stained for all lysosomal measurements. For the LysoLive Assay (Marker Gene Technologies, Inc.; M27745), the β-glucosidase specific substrate GlucGreen was incubated at 5 μM in media for 30 minutes at 37° C. Cells were washed three times with ice-cold PBS and subsequently fixed in 4% PFA for 15 minutes at room temperature. Slides were mounted with ProLong™ Gold Antifade Mountant with DAPI (ThermoFisher; P36935).

For the SiR-Lysosome Assay (Cytoskeleton, Inc.; CYSCO12), a cell-permeable peptide conjugated to a silicon rhodamine (SiR) dye was incubated in human PAECs as a measure of active cathepsin D. SiR-Lysosome was incubated with cells at 1 μM and with the calcium channel blocker verapamil at 1 μM to improve signal intensity for 30 minutes at 37° C. Cells were rinsed three times with ice-cold PBS, fixed in 4% PFA, and mounted as described above.

Assessment of lysosomal acidification: Lysosomal acidification was measured using the LysoSensor™ Yellow/Blue DND-160 (PDMPO) dye (ThermoFisher; L7545). The LysoSensor™ Yellow/Blue DND-160 (PDMPO) dye exhibits dual-excitation (i.e., 329 and 384 nm) and dual-emission (i.e., 440 and 540 nm) spectral peaks that are pH-dependent (pKₐ 4.2). In acidic organelles, the dye has predominantly yellow fluorescence. In basic organelles, the dye has predominantly blue fluorescence. The unique spectral properties of this dye allow for ratiometric quantification.

Live human PAECs were incubated with 1 μM of dye in 0.1% FBS cell-specific media for 1 minute at 370C. Cells were rinsed with PBS, trypsinized, pelleted in polystyrene tubes at 300 g for 5 minutes, and rinsed twice more with PBS. Cells were immediately analyzed on a BD LSRFortesssa™ Flow Cytometer (BD Biosciences) at the Unified Flow Core at the University of Pittsburgh. Median fluorescent intensity (MFI) ratio was calculated by using the yellow over the blue MFI fluorescent values.

Assessment of lysosomal lipid content: Neutral lipids were stained with the fluorescent dye 4,4-difluoro-1,3,5,7,8-pentamethyl-4-bora-3a,4a-diaza-s-indacene (BODIPY®; ThermoFisher; D3922). Acidic organelles (i.e., lysosomes) were stained with LysoTracker™ Red DND-99 (ThermoFisher; L7528). Live human PAECs were incubated in cell-type specific media containing 1 μM BODIPY® and 50 nM LysoTracker™ Red DND-99 for 30 minutes at 37° C. Cells were then washed with PBS three times before fixation with 4% PFA for 30 minutes at room temperature. Cells were rinsed with PBS three more times and then mounted with ProLong™ Gold Antifade Mountant with DAPI (ThermoFisher; P36935). Images were acquired on a Nikon A1 Confocal Microscope at the Center for Biologic Imaging at the University of Pittsburgh.

Lysosomal lipid content was measured by the degree of colocalization between BODIPY® (i.e., neutral lipids) and LysoTracker™ Red DND-99 (i.e., acidic organelles). Colocalization was measured using EzColocalization in Fiji and quantified as Pearson's Correlation Coefficient.

Targeted LC-MS for cholesterol intermediates and oxysterols: Human PAECs were treated and collected for cholesterol intermediates and oxysterol analyses at 1×10⁶ cells per glass 16×125 mm tube (Pyrex; 9826). Cells were subjected to a liquid-liquid extraction of sterols. Briefly, 1 mL of dichloromethane, methanol, and water were added to each sample. The sample was then vortexed and centrifuged to produce two liquid phases. The lower phase was carefully transferred to a new glass tube and dried under nitrogen. Samples were then resuspended in hexane and the resultant lipid species were analyzed by liquid chromatography-mass spectrometry (LC-MS). Samples were run on the SCIEX QTRAP 6500+ equipped with a Shimadzu LC-30AD HPLC system and a 150×2.1-mm, 5 μm Supelco Ascentis silica column. The LC-MS data was analyzed using MultiQuant (SCIEX).

Statistical analyses of the UPMC and STRIDE cohorts: For the UPMC cohort, the effect of SNP rs11154337 G minor allele on six-minute walk distance was calculated using a dominant genetic model. In addition, the effect of the minor allele on time to death or last follow-up was tested using a Cox proportional hazard model. In both models, the genetic effect was adjusted for sex, age, comorbidities, and vasodilator therapies. Stata 17.0 pymol was used for these analyses.

For the STRIDE cohort, analysis began with raw data in the plink format. Strand information was converted using update_build.sh to InfiniumOmniExpress-24v1-3_A1-b37.strand. Next, shapeit was utilized to phase and impute2 with 1000G_Phase3 b37 for imputation. Chromosome bins were merged, and redundant SNPs removed using gtool. First pass of survival analysis was performed with R package gwasurvivr performing a coxph test for each SNP versus Time to Death (FinalEvent) including the covariates: sex, age, PAH type, WHO classification, study inclusion (Encysive or Prospective), AnyDrugsBefore, UsePDE, UsePros, UseWarf, UseOxy and a maf filter of 0.005. Further analysis was performed testing SNP rs11154337 against Time to Death (FinalEvent) in patients of European descent (EthConEUR) based on self-reported ethnicity and discriminant principal component analysis.

Staining for neutral lipids: Neutral lipids were stained in cells using the fluorescent dye 4,4-difluoro-1,3,5,7,8-pentamethyl-4-bora-3a,4a-diaza-s-indacene (BODIPY®). Live cells were grown on glass coverslips in plasticware. Cells were rinsed three times with PBS to remove residual media. A staining solution of 2 μM BODIPY® in PBS was applied to cells for 15 minutes at 37° C. Cells were rinsed three times with PBS before fixing with 4% paraformaldehyde for 15 minutes at room temperature. Coverslips were mounted with ProLong™ Gold Antifade Mountant with DAPI (ThermoFisher; P36935). Images were acquired on a Nikon A1 Confocal Microscope at the Center for Biologic Imaging at the University of Pittsburgh.

Measurement of cholesterol uptake: To assess cholesterol uptake, a Cholesterol Uptake Assay Kit was utilized per the manufacturer's specifications (abcam; ab236212). Briefly, treated human PAECs were incubated in 0.1% serum, cell-type specific media with supplemented fluorescent, NBD-cholesterol at 20 μg/mL for 24 hours. Cells were rinsed with PBS, trypsinized, pelleted in polystyrene tubes at 300 g for 5 minutes, and rinsed twice more with PBS. Cells were immediately analyzed on a BD LSRFortesssa™ Flow Cytometer (BD Biosciences) at the Unified Flow Core at the University of Pittsburgh. Flow cytometric analysis was chosen over confocal microscopy due to the high rate of photobleaching observed with NBD-cholesterol.

Assessment of cholesterol content: To assess cholesterol content, the Cholesterol/Cholesterol Ester-Glo™ Assay Kit was utilized (Promega; J3190). This assay measures cholesterol using a cholesterol dehydrogenase that links the presence of cholesterol to NADH production and thus proluciferin activation. Human PAECs were plated at a density of 20,000 cells per well in a 96-well plate in replicates of six. The assay was performed as the manufacturer specifies.

Apoptosis measured via caspase-3/7 activity: Apoptosis was assessed using the Caspase-Glo® 3/7 Assay System (Promega; G8090). This assay functions by providing a luminogenic, caspase-3/7 substrate optimized for caspase activity. Cleavage of this substrate generates a luminescence-based signal through luciferase. Equal volumes of this substrate were added to wells containing human PAECs (5,000 per well) and left to incubate at room temperature for 30 minutes. Luminescence was measured via spectrophotometry. Luminescent signal was normalized to protein content per well, assessed using the Pierce™ BCA Protein Assay Kit (ThermoFisher; 23227).

Proliferation measured via BrdU incorporation: Proliferation was assessed using the BrdU Cell Proliferation Assay Kit (Cell Signaling Technology; 6813). This assay functions by measuring 5-bromo-2′-deoxyuridine (BrdU) into proliferating cells using an anti-BrdU antibody. BrdU was added to complete growth media containing 5% serum for two hours. Human PAECs (5,000 per well) were fixed and denatured before application of the mouse anti-BrdU antibody. Next, anti-mouse HRP-linked antibody was added. A development substrate was then added to detect with HRP-linked, antibody complexes to BrdU. Absorbance was measured at 450 nm using spectrophotometry.

Leukocyte and monocyte adhesion assays: Immune activation of the endothelium was assessed by measuring the adhesion of immune cells to an endothelial monolayer. Human PAECs were cultured until a complete monolayer was formed. Immune cells were stained with either CellTrace™ Blue or CFSE (ThermoFisher; Blue, C34568; CFSE, C34554) per the manufacturer's protocol. Between 2.0 to 2.5×10¹ stained immune cells were added to each well of a six-well plate and allowed to incubate for 24 hours. Wells were then rinsed two times with PBS and subsequently fixed with 4% PFA for 15 minutes at room temperature. After fixation, the cells were rinsed once more with PBS. Fluorescent images were acquired at 4× magnification for each well. Immune cell number per image was quantified using Fiji. For the leukocyte adhesion assay, HuT 78 cutaneous T lymphocytes were used (ATCC). For the monocyte adhesion assay, THP-1 peripheral blood monocytes were used (ATCC).

Application of oxysterols and bile acids: Oxysterols and bile acids were applied to human PAECs in 0.1% FBS, cell-specific media. 25-hydroxycholesterol and 7HOCA were dissolved in 100% ethanol and applied to cells at a concentration of 25 μM or 50 μM for 24 hours, respectively.

Animal studies: All animal studies were approved by the Division of Laboratory Animal Resources at the University of Pittsburgh. The Ncoa7 knockout mouse line (C57BL/6 Ncoa7tm1.1(KOMP)Vlcg) was obtained from the Knockout Mouse Project (KOMP; komp.org) and generated using sperm for rederivation at the Genome Editing, Transgenic, & Virus Core at Magee Women's Research Institute. Obtained mice were bred in-house to generate homozygous, Ncoa7 knockout mice. To elicit a model of pulmonary inflammation resulting in severe PH, Ncoa7 knockout mice were crossbred with C56BL/6 Il6 transgenic (Tg⁺) mice. The Il6 Tg⁺ mice contain a Clara cell 10-kD promoter (CC10) that drives constitutive expression of IL-6 within the lung (M. K. Steiner et al., Interleukin-6 overexpression induces pulmonary hypertension. Circ Res 104, 236-244, 228p following 244 (2009)). C57BL/6 mice were used for orotracheal delivery of either PBS or 7HOCA (10 mg/kg) serially injected every 5 days for 4 weeks under chronic hypoxia (10% O₂). Mice were taken to 15 weeks of age under normal oxygen tension before echocardiography, invasive hemodynamics measurement, and tissue harvesting. A monocrotaline rat model of PAH was utilized with a single injection of monocrotaline (80 mg/kg) at 8 to 9 weeks of age. Rats were then injected intraperitoneally with DMSO or 958ₐₘᵢ (7.5 mg/kg) for 10 days post-monocrotaline loading dose before takedown.

Hemodynamic measurements: Echocardiography was performed on 15-week-old mice using a 15-45 MHz transthoracic transducer and a VisualSonics Vevo770 system (Fujifilm). Anesthesia was administered with 2% isoflurane in 100% O₂ during animal positioning and hair removal, and subsequently decreased to 0.8% isoflurane during image acquisition. Data were analyzed in a blinded manner by a technician.

For right heart catheterization, mice were given ketamine/xylazine (9:1; Henry Schein) or subjected to isoflurane (Henry Schein). The isoflurane vaporizer was maintained at 1.5 to 2% with an oxygen gas flow rate of 1 L/min. Right ventricular systolic pressure was measured with Millar catheters (SPR-513 and SPR-671). Catheters were inserted into the jugular vein and then guided through the right atrium and into the right ventricle. Steady right ventricular systolic pressure waveforms were measured for two minutes. Analysis of waveforms were performed in a blinded manner.

Structural modelling of the catalytic domain of NCOA7ₛₕₒᵣₜ: The sequence of NCOA7ₛₕₒᵣₜ (isoform 5; Q8N108-5) was downloaded from UniProt. Its catalytic domain (P55-D219) was modeled using SWISS-MODEL and was based on the crystal structure of the TLDc domain of oxidation resistance protein 2 (OXR2) from zebrafish (PDB ID 4ACJ). The sequence identity between the catalytic domains of NCOA7 and OXR2 was calculated as 61.8% using Clustal Omega, which implies that the two domains share the same structure.

Gaussian network model (GNM) analyses: In a GNM analysis, the protein structure is represented as an elastic network, where residues serve as nodes of which the positions are identified by those of the α-carbons. As such, the GNM for NCOA7 was developed using a total of 165 residues. The overall potential was represented as the sum of the harmonic potentials between pairs of nodes within an interaction ranged defined as a C^{α}—C^{α} distance less than 7.3 Å. The resultant topology of the network was recorded in a N×N Kirchhoff matrix. All computations were performed using the ProDy API.

Druggability simulations and analyses: Druggability simulations were performed for NCOA7 in the presence of the probe molecules, using the all-atom MD simulation package NAMD with the CHARMM36 force field for proteins, the TIP3P water model, and the CGenFF force field for the probe molecules. Probe molecules were benzene, isobutane, imidazole, acetamide, isopropanol, isopropyl amine, and acetate, and were derived from the statistical evaluation of chemical/functional groups most frequently observed in FDA-approved drugs. The trajectories were analyzed using the DruGUI module of ProDy and, six independent runs of 40 ns were performed. All MD snapshots were superposed onto the reference PDB structure using C^{α}-atoms and a cubic grid-based representation of the space with the grid edge size set to 0.5 Å. Probe molecules whose non-hydrogen atoms were within 4.0 Å from protein atoms were considered to interact with the protein. For each probe type, the individual occupancy of grids was calculated using their centroids. We evaluated the occupancy of each probe for each voxel and quantified binding energies using the inverse Boltzmann relation. The resulting binding free energy map identified interaction spots with low (favorable) energy for one or more probe types, and high occupancy voxels were called druggable hot spots.

Pharmacophore modeling: Using Pharmmaker, we identified the residues involved in high affinity interactions with each molecular probe type. The residue-probe interactions were subsequently rank-ordered based on their frequency of occurrence at the druggable hot spots in multiple runs. A snapshot that simultaneously exhibited residue-probe pairings of the probability (e.g., N62-benzene, E66-isopropylamine, P80-benzene, and W81-benzene) was selected as template to construct the pharmacophore model. The pharmacophore model contained a hydrogen bond donor and a hydrophobic feature at the isopropylamine site and hydrophobic and aromatic rings at the two benzene sites. The pharmacophore model was then screened against the ZINC and MolPort libraries using Pharmit. The MolPort library contains 67,033,884 conformers corresponding to 4,848,718 compounds, and the ZINC library contains 122,276,899 conformers of 13,127,550 compounds. Among top-scoring compounds, MolPort-004-267-958 was selected for further refinement after initial experimental validation.

Molecular dynamics simulations of 958 and 958ₐₘᵢ: All atom MD systems were set-up using GHARMM-GUI Solution Builder. Simulations were performed using the MD simulation package NAMD with the CHARMM36m force field for proteins, the TIP3P water model, and the CGenFF force field for the compounds. Three independent runs of 0.2 ms (for a total 0.6 ms) were performed for 958 and 958ₐₘᵢ. The system in each case comprised the NCOA7 protein and the compound in the presence of explicit water and 0.15 M NaCl, relaxed using the equilibration steps in CHARMM-GUI. We performed NPT dynamics for 0.2 ms with 2 fs time steps, and constant pressure (1 bar) and temperature (300 K). Contact duration between target and compound and hydrogen bond formation were analyzed using VMD 1.9.4. Data visualization was performed using PyMOL 2.3.5 and GNUPlot. Binding affinities were calculated using PRODIGY-LIG.

Statistics: All in vitro data represent at least three independent experiments. The number of animals used for a given experimental model was calculated to measure at least a 20% difference between the means of the control and experimental groups with a power of 80% and a standard deviation of 10%. The number of patient samples used for molecular analyses was primarily determined by clinical availability. Shapiro-Wilks testing was used to determine normality of data distribution. For normally distributed data, paired data were analyzed with a two-tailed Student's t-test and grouped data were compared with either a one- or two-way analysis of variance with post hoc Tukey analysis to adjust for multiple comparisons. Significance was determined by a Pvalue less than 0.05. All data are presented as mean±standard deviation.

### Results

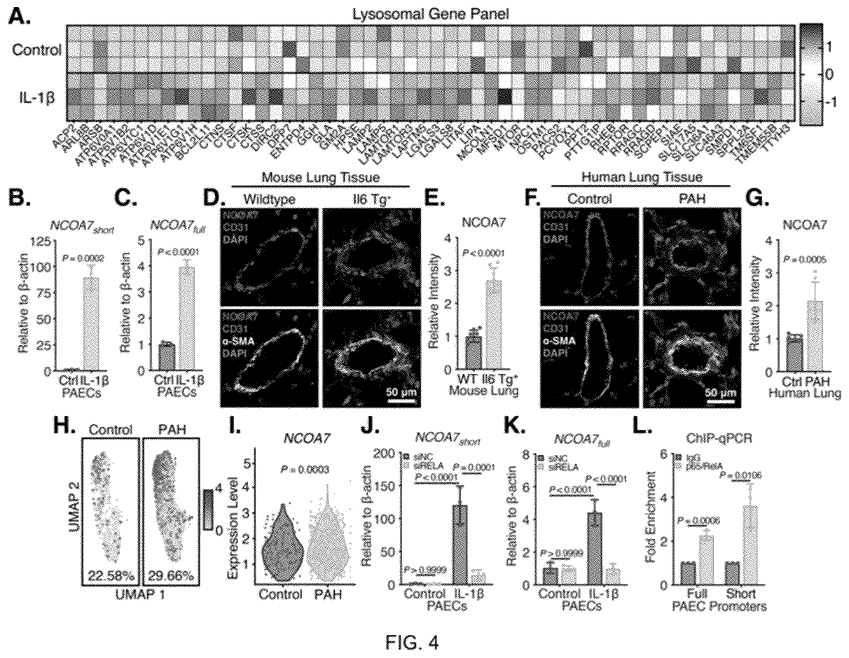

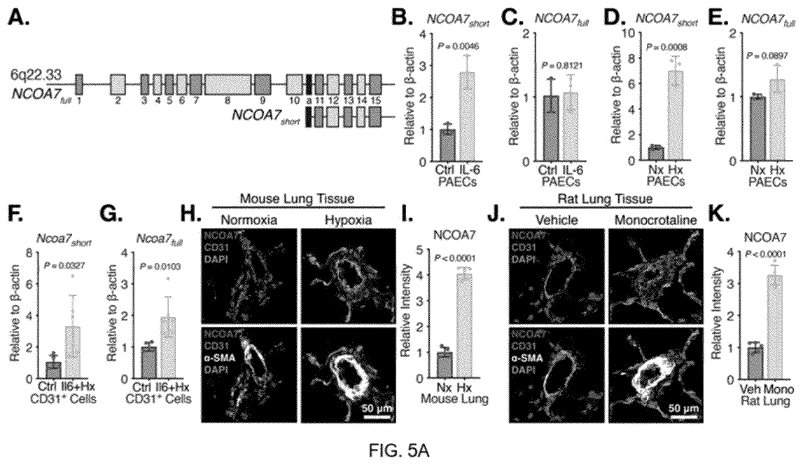

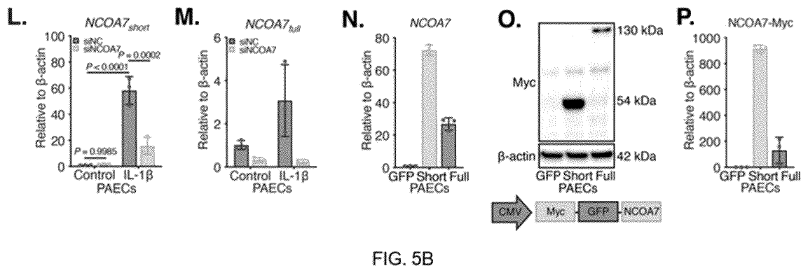

Convergent inflammatory regulation of NCOA7 across cellular, animal, and human instances of PAH: An unbiased, transcriptomic analysis was performed on primary human pulmonary artery endothelial cells (PAECs) exposed to IL-1β—a proinflammatory cytokine elevated in the plasma of PAH patients and known trigger of disease pathogenesis. Lysosomal regulatory genes were globally upregulated with a distinct subset comprising V-ATPase subunits, which are binding partners of NCOA7 that promote acidification of the lysosome (FIG. 4 (A)). Correspondingly, IL-1β upregulated NCOA7—both the canonical, full-length isoform (NCOA7_{full}) and, to a greater extent, an alternative-start, short-length version of NCOA7 (NCOA7ₛₕₒᵣₜ) (FIG. 4 (B,C), FIG. 5A (A)).

Other triggers of EC dysfunction in PAH similarly upregulated NCOA7. Specifically, exposure to the proinflammatory cytokine IL6 and its soluble receptor (IL6Rα), which has been linked to PAH, induced both short- and full-length isoforms (FIG. 5A (B,C)). Hypoxia, a well-established driver of PH, also increased both isoforms (FIG. 5A (D,E)). Taken together, these data indicate a potential role for NCOA7, especially its unique, alternative-start isoform, across multiple triggers of PH.

Using a severe inflammatory rodent model of PAH, transgenic mice under constitutive IL-6 overexpression and chronic hypoxia demonstrated elevated Ncoa7 expression in CD31⁺ ECs isolated from lung tissue (FIG. 5A (F,G)). This was also observed in the pulmonary vessels of Il6 transgenic mice without hypoxia—a milder form of experimental PAH (FIG. 4 (D,E)). Similarly, examining the in situ localization of NCOA7, there was marked and transmural upregulation in pulmonary vessels with a heightened expression localized to the endothelium—in both the chronic hypoxia mouse model and the monocrotaline-exposed PAH rat model (FIG. 5A (H-K).

Moreover, human lung tissue from patients with Group I PAH revealed elevated NCOA7 expression within pulmonary vessels when compared to healthy controls (FIG. 4 (F,G)). Single-cell RNA sequencing performed on lungs from idiopathic Group I PAH patients revealed an increased number of ECs expressing NCOA7 when compared to healthy controls (22.58% versus 29.66%) (FIG. 4 (H). Moreover, in NCOA7-positive ECs, NCOA7 expression was upregulated in PAH patients (FIG. 4 (I)). Thus, in proinflammatory models of PH using primary PAECs, rodent models, and human patients, we found that NCOA7 was upregulated within the pulmonary vessel and, most importantly, the endothelium. However, since NCOA7 deficiency and consequent loss of lysosomal acidification should increase inflammation and worsen disease in other contexts, NCOA7 is believed to act as a homeostatic brake under proinflammatory stress to reduce disease pathogenesis through attenuation of EC immunoactivation.

To investigate upstream inflammatory mechanisms that modulate NCOA7, binding sites for the well-established inflammatory transcription factor complex—the RelA/p65 (RELA) subunit of NF-κB—were predicted in the canonical (i.e., full-length) and non-canonical (i.e., short-length) NCOA7 promoter regions. Correspondingly, RELA knockdown in PAECs abrogated the IL-1p-mediated upregulation of both isoforms (FIG. 4 (J,K)). A chromatin immunoprecipitation coupled with quantitative PCR (ChIP-qPCR) revealed significant enrichment of ReIA/p65 at DNA sequences of the canonical and non-canonical promoters (FIG. 4 (L)), indicating direct promoter-transcription factor binding.

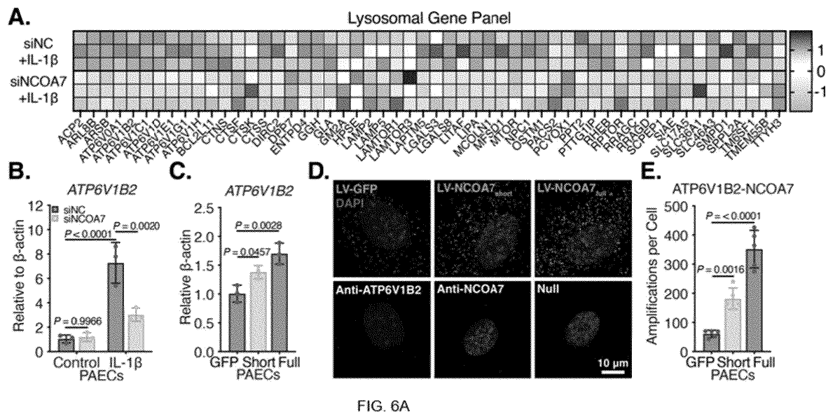

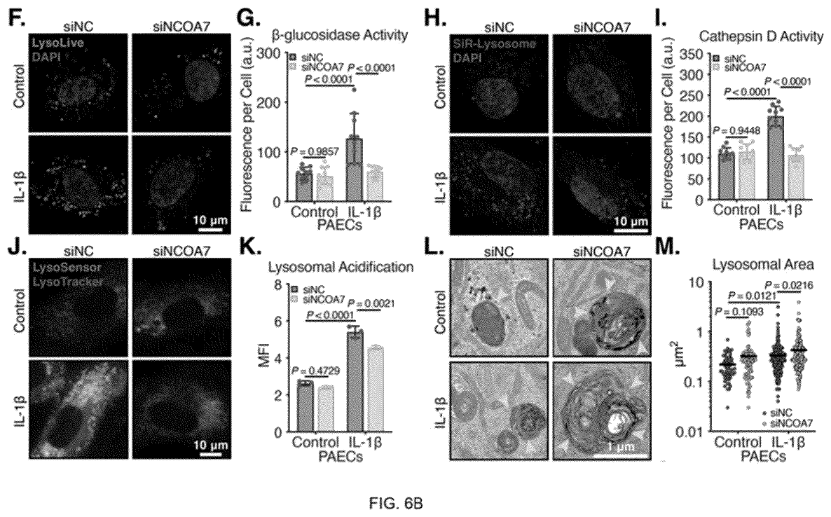

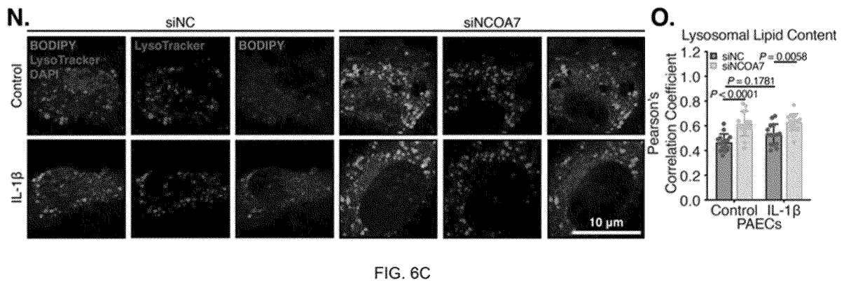

Loss of NCOA7 promotes lysosomal dysfunction and lipid accumulation: To investigate a putative link between NCOA7 and oxysterol production in the presence of proinflammatory conditions, the NCOA7-mediated control of lysosomal acidification was characterized, given the known function of lysosomes in sterol trafficking. In human PAECs exposed to IL-1β, knockdown of NCOA7 reversed the interleukin-specific alteration of network of genes governing lysosomal function (FIG. 6A (A)). Notably, a number of these genes (e.g., ATP6V0A1, ATP6V1B2, ATP6V1C1, ATP6V1D, ATP6V1E1, ATP6V1G1, and ATP6V1H) encode for subunits of V-ATPases-machinery necessary for lysosomal acidification and thus the function of pH-sensitive enzymes.

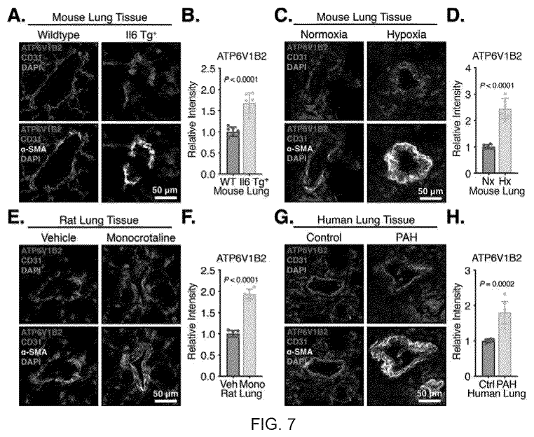

Proteomics-based studies established that NCOA7 interacts with ATP6V1B1—a renal specific paralog of ATP6V1B2. Correspondingly, in PAECs, NCOA7 knockdown abrogated the IL-1β-mediated upregulation of ATP6V1B2, and the forced overexpression of either the short- or full-length isoforms upregulated ATP6V1B2 (FIG. 6A (B,C)). In addition, ATP6V1B2 was upregulated in the pulmonary endothelium of rodent and human models of PH (FIGS. 7A and 7B (A-H)). To assess for a direct interaction between these proteins, a proximity ligation assay demonstrated perinuclear staining indicative of ATP6V1B2-NCOA7 interactions and consistent with a perinuclear distribution of lysosomes. Moreover, forced overexpression of NCOA7ₛₕₒᵣₜ or NCOA7_{full} upregulated the number of ATP6V1B2-NCOA7 interactions in the lysosome (FIG. 6A (D,E)). These data demonstrated a role for both short- and full-length NCOA7 as regulatory components of the V-ATPase complex with putative downstream lysosomal function.

To assess NCOA7 activity in modulating lysosomal acidification, two quantitative measures of lysosomal enzyme activity were utilized. First, cleavage of the fluorescent LysoLive tracer was increased by IL-1β and subsequently blocked with loss of NCOA7 (FIG. 6B (F,G)). Second, lysosome-dependent cathepsin D activity was assessed utilizing SiR-Lysosome. In line with the IL-1β-mediated increase of ATP6V1B2, cathepsin D activity was significantly upregulated by IL-1β, as noted by SiR-Lysosome fluorescence, and was abrogated by NCOA7 knockdown (FIG. 6B (H,I)).

To directly evaluate lysosomal acidification, the acidotropic probe LysoSensor Green DND-189 was utilized for its accumulation in acidic compartments and enhanced fluorescence under acidic conditions. Consistent with the IL-1β-mediated increase in lysosomal enzyme activity, IL-1β increased the LysoSensor fluorescent signal, which was reversed with NCOA7 knockdown (FIG. 6B (J)). Additionally, IL-1β drove a shift to yellow fluorescence in PAECs when using the acidotropic probe LysoSensor Yellow/Blue DND-160, indicating enhanced acidification of the lysosomal lumen (FIG. 6B (K)). The addition of NCOA7 deficiency reversed the IL-1β-mediated yellow fluorescent shift. These observations mimic findings in LSDs, which are notable for the accumulation of undigested cellular components in the lysosomal compartment.

Failure of such V-ATPase complex formation or lysosomal acidification is a known driver of lysosomal dysmorphology. Accordingly, morphologic analysis of lysosomes using transmission electron microscopy of human PAECs deficient for NCOA7 revealed marked hypertrophy of lysosomes as quantified by lysosomal area (FIG. 6B (L,M)), denoting an inability of the lysosomal compartment to breakdown and process cellular components. Moreover, the enlarged lysosomes in NCOA7-deficient cells carried lamellar-like inclusions (FIG. 6B (L,M); yellow arrows), indicative of lipid buildup-again phenocopying LSDs that present with abnormal lysosomal lipid accumulation.

To address whether the lamellar-like structures observed in the electron micrographs were lipids, PAECs were co-stained using a dye against neutral lipids (i.e., BODIPY®) and a dye that specifically localizes to acidic compartments (i.e., LysoTracker). With loss of NCOA7, there was an increase in lipid punctae throughout the cell, which specified vesicular accumulation. Correspondingly, the hyperintense lipid punctae were identified within acidic vesicles, supporting the idea that loss of NCOA7 caused such lysosomal accumulation of lipids (FIG. 6C (N,O)). Taken together, these findings establish NCOA7 as a binding partner to the V-ATPase complex to promote lysosomal acidification and sterol trafficking in PAECs.

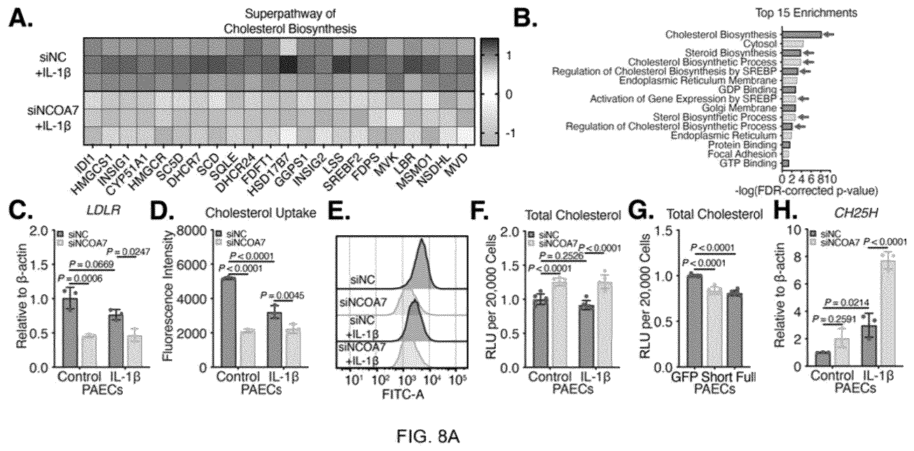

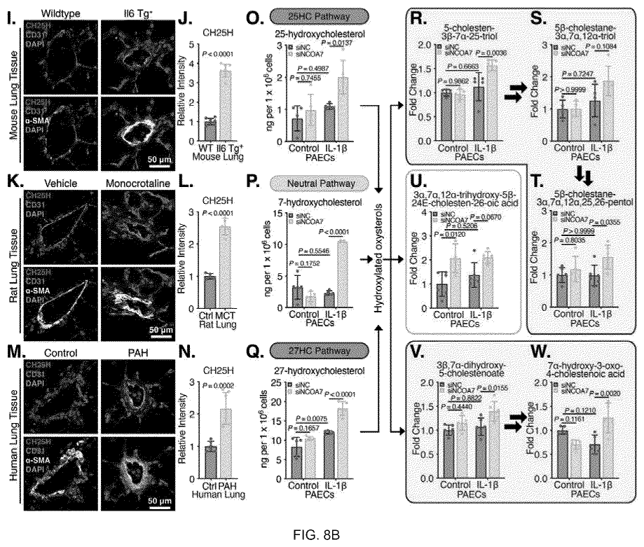

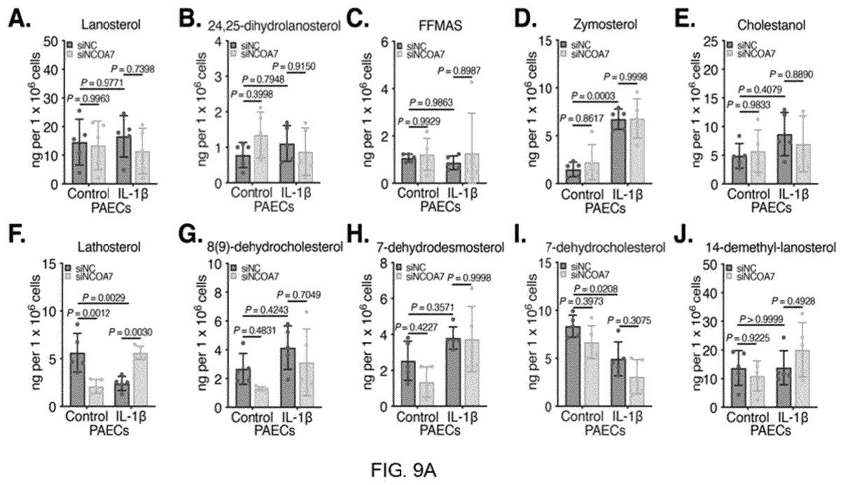

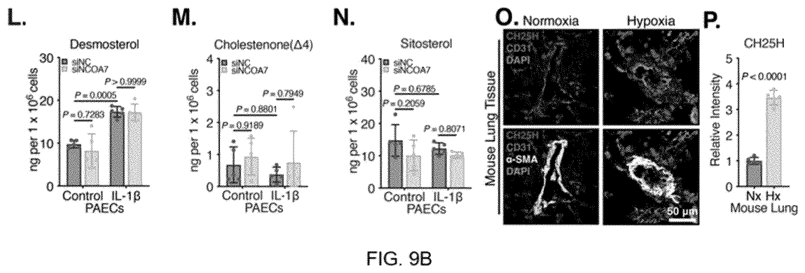

Deficiency of NCOA7 reprograms sterol metabolism via abnormal lipid accumulation: Alterations of lysosomal lipid trafficking affect sterol homeostasis. Correspondingly, transcriptomic analysis of NCOA7-deficient human PAECs revealed marked enrichment and downregulation of biosynthetic processes related to sterol metabolism (FIG. 8A (A,B); red arrows). In a sterol saturated cell, uptake pathways of extracellular cholesterol are inhibited, specifically through reduction of the low-density lipoprotein receptor (LDLR) density on the cell membrane. NCOA7 deficiency in PAECs reduced LDLR expression (FIG. 8A (C)), accompanied by a functional attenuation in the uptake of fluorescently labeled cholesterol (FIG. 8A (D,E)). Total cholesterol content in NCOA7-deficient PAECs was also upregulated, while forced overexpression of NCOA7 reduced total cholesterol content (FIG. 8A (F,G)). No appreciable differences were detected in post-squalene intermediates in NCOA7-deficient versus NCOA7-replete PAECs (FIGS. 9A and 9B (A-N)), indicating that NCOA7-dependent modulation of sterol intermediate flux does not rely upon de novo cholesterol synthesis. Thus, the downregulation of sterol metabolism by NCOA7 deficiency is driven primarily by lysosomal alterations of sterol handling rather than by de novo synthesis.

To protect against cholesterol accumulation, the cell can engage either in its direct export through transporters or by increasing its solubility through a series of oxidative steps. Accordingly, deficiency of NCOA7 significantly upregulated cholesterol 25-hydroxylase (CH25H)—an oxysterol-generating enzyme that increases cholesterol solubility (FIG. 8A (H)). Revealing the in vivo relevance of these processes, CH25H was upregulated in the pulmonary vessels of proinflammatory rodent models of PAH and Group I PAH patients with localization to the endothelium (FIG. 8B (I-N) and FIG. 9B (O,P)). Overall, these data established a central role for NCOA7 in the maintenance of EC sterol homeostasis through the generation of oxidized sterol species in cultured PAECs and in diseased endothelium in vivo.

NCOA7 deficiency induces endothelial generation of oxysterols and downstream bile acid derivatives: To corroborate whether the observed upregulation of CH25 Henhanced oxysterol production, targeted lipidomic analysis was performed using liquid chromatography-mass spectrometry (LC-MS). NCOA7 knockdown in human PAECs exposed to IL-1β significantly upregulated 25-hydroxycholesterol (25HC), 27-hydroxycholesterol (27HC), and autoxidation-generated 7α-hydroxycholesterol (FIG. 8B (O-Q)). These oxysterols are known to be metabolized into downstream bile acid derivatives through incompletely understood mechanisms. Accordingly, NCOA7 knockdown upregulated several downstream bile acid derivatives in sequential pathways, such as 5-cholesten-3β-7α,25-triol, 5β-cholestane-3α,7α,12α-triol, and 5β-cholestane-3α,7α,12α,25,26-pentol (FIG. 8B (R-T)). Moreover, the upstream metabolites 3β,7α-dihydroxy-5-cholestenoate and 7α-hydroxy-3-oxo-4-cholestenoic acid (7HOCA) were also upregulated (FIG. 8B (V,W)). Thus, consistent with the upregulation of the oxysterol-generating enzyme CH25H (FIG. 8A (H)), NCOA7 deficiency induced production of the numerous oxidized cholesterol metabolites and downstream bile acids in ECs.

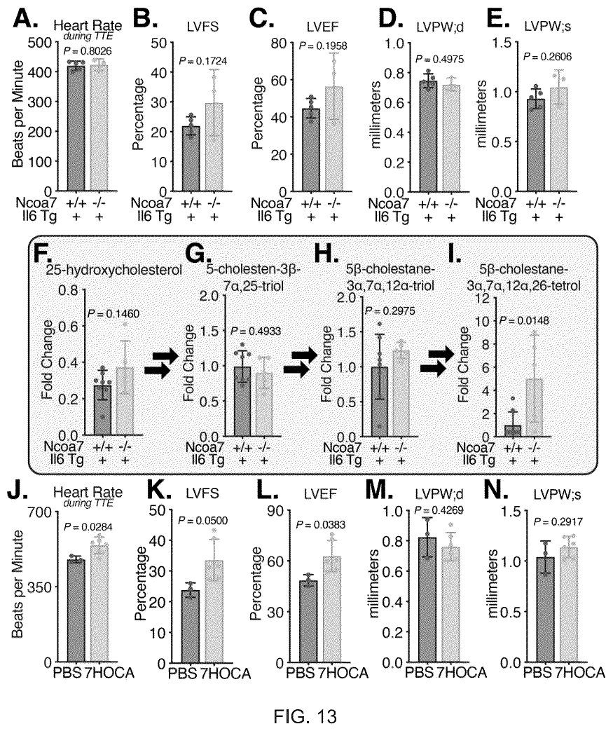

Oxysterols and bile acids as markers of morbidity and mortality in PAH: Emphasizing the clinical importance of this mechanism in controlling disease severity, a plasma signature was identified inclusive of the same NCOA7-dependent sterols and bile acids associated with PAH mortality (adjusted P<1.1×10⁻⁶). In a companion study, via an unbiased, metabolome-wide association study from the multicenter PAH Biobank cohort (N=2,796), 13 distinct plasma oxysterols and bile acids were identified that best predicted four-year mortality in PAH. Notably, of these top 13 oxysterols and bile acids, four were the same metabolites upregulated in ECs deficient for NCOA7 (FIG. 8B (Q,R,S,W) and in the serum of Ncoa7-deficient mice (FIG. 13 (I)). These findings emphasize the clinical importance of NCOA7-dependent oxysterols and bile acids in controlling PAH severity, thereby establishing a framework that links lysosome-dependent inflammation with both population-level and metabolome-wide signals in PAH.

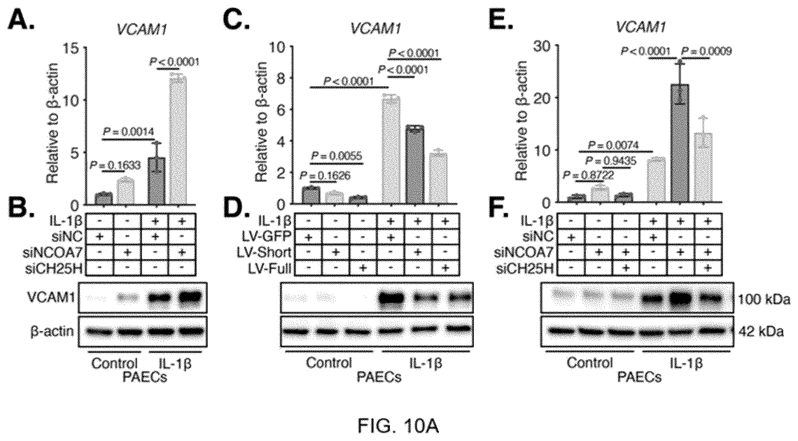

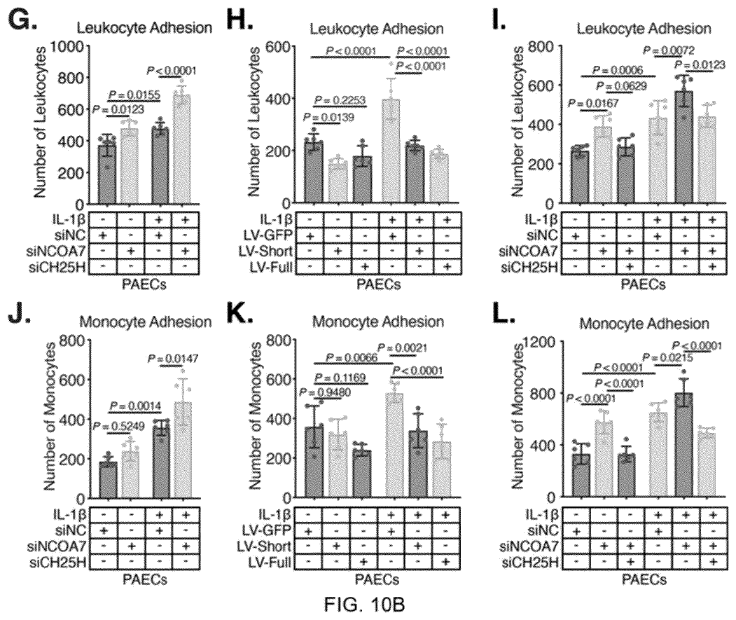

NCOA7 deficiency promotes endothelial dysfunction through oxysterol generation: Given the immunomodulatory functions of oxysterols in diseased endothelium, we sought to determine if NCOA7 deficiency relied upon oxysterols to promote EC dysfunction. NCOA7 deficiency upregulated the vascular cellular adhesion molecule 1 (VCAM1)—a surrogate of endothelium immunoactivation (FIG. 10A (A,B)). Conversely, forced overexpression of NCOA7 isoforms reversed VCAM1 expression (FIG. 10A (C,E)). To determine if NCOA7 deficiency was dependent upon downstream, oxidized forms of cholesterol to induce PAEC pathophenotypes, concomitant knockdown experiments against the oxysterol generating enzyme CH25H, which is upregulated with NCOA7 deficiency, were performed. Notably, inhibition of CH25H induction under NCOA7 deficiency prevented VCAM1 expression (FIG. 10A (E,F)).

In line with observed VCAM1 changes under various states of NCOA7 and CH25H expression, we observed increased attachment of both leukocytes and monocytes to an EC monolayer deficient for NCOA7 (FIG. 10B (G,J)), which could be reversed with NCOA7 overexpression (FIG. 10B (H,K)), and a significant attenuation of immune cell attachment with inhibition of CH25H under conditions of NCOA7 deficiency (FIG. 10B (I,L)).

In addition and consistent with the concept of an immunoactivated, apoptosis-resistant, and hyperproliferative endothelium in pulmonary vascular disease, NCOA7 deficiency in PAECs abrogated IL-1β-mediated apoptosis while simultaneously enhancing proliferative capacity (FIG. 11A (A,D)). NCOA7 facilitated PAEC apoptosis under proinflammatory conditions (FIG. 11A (B)) and, in parallel, attenuated proliferation with inhibition more pronounced under IL-1β (FIG. 11A (E)). Inhibition of CH25H upregulation, however, reversed the attenuation of apoptosis, while enhancing the proliferative capacity of the cells (FIG. 11A (C,F)). In sum, the presence of NCOA7 prevented immunoactivation of the endothelium with induction of apoptosis and inhibition of proliferation.

To determine if bile acids are sufficient to immunoactivate the endothelium, 7HOCA was directly applied to PAECs in culture. Displaying its proinflammatory nature, 7HOCA significantly upregulated VCAM1 (FIG. 10C (M,N)), thus promoting adhesion of both leukocytes and monocytes to a PAEC monolayer (FIG. 10C (O,P)). Notably, the direct application of 25HC and downstream derivatives, such as a triol and tetrol, similarly upregulated VCAM1 and enhanced immune cell adhesion to a PAEC monolayer (FIG. 11B (G-O)). In sum, these findings demonstrate that oxysterol-generating enzymes and their downstream oxidized sterol species are necessary and sufficient in mediating immune activation of the NCOA7-deficient pulmonary endothelium.

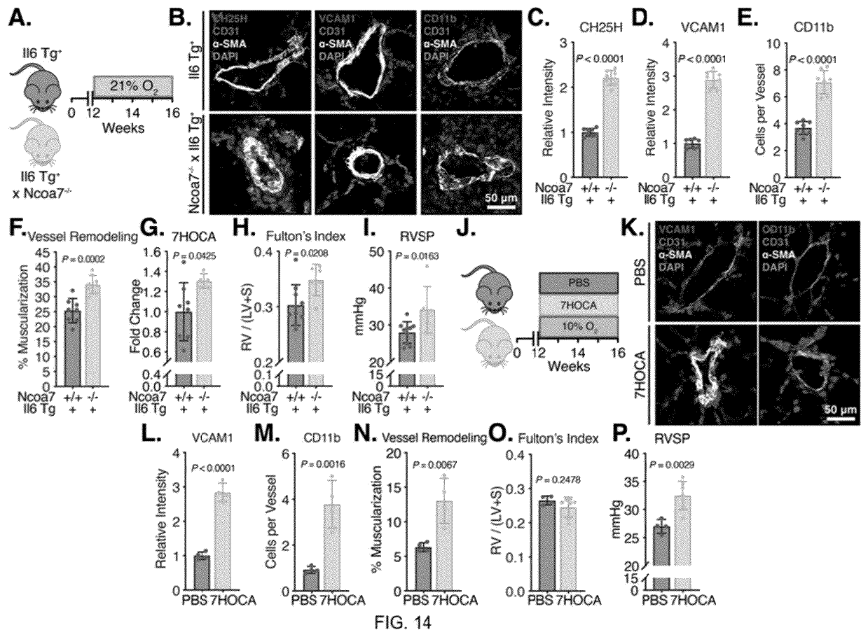

Loss of NCOA7 and the orotracheal delivery of 7HOCA worsens PAH in vivo: To determine if the presence of NCOA7 protects against EC immunoactivation in PAH severity, an 116 transgenic (Tg⁺) mouse was used to elicit severe pulmonary inflammation as a model of angioproliferative PAH (M. K. Steiner et al., Interleukin-6 overexpression induces pulmonary hypertension. Circ Res 104, 236-244, 228p following 244 (2009)). A whole-body knockout mouse for Ncoa7 was crossed onto Il6Tg⁺ mice to determine if loss of NCOA7 would worsen indices of PH in vivo (FIG. 14 (A)). Echocardiographic assessment excluded any gross alterations in left ventricular function, as noted by left ventricular fractional shortening (LVFS), left ventricular ejection fraction (LVEF), and left ventricular posterior wall distance during diastole and systole (LVPW;d and LVPW;s) (FIG. 13A (A-E)).

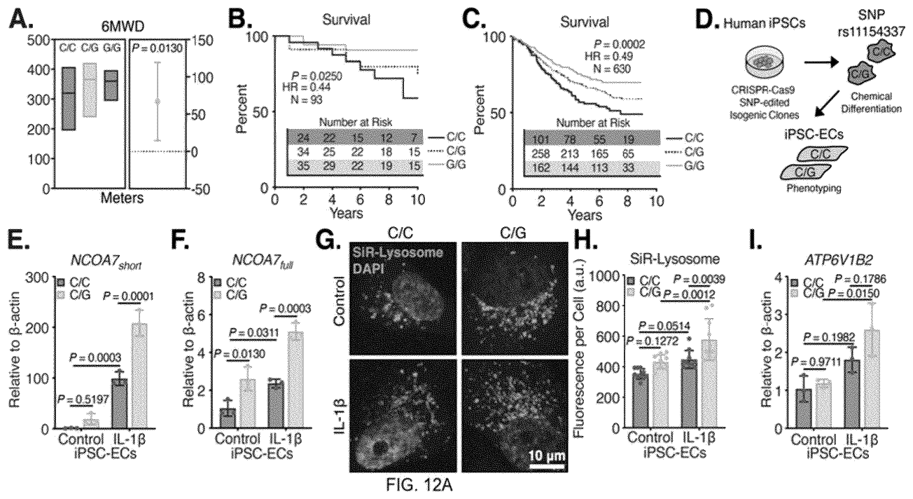

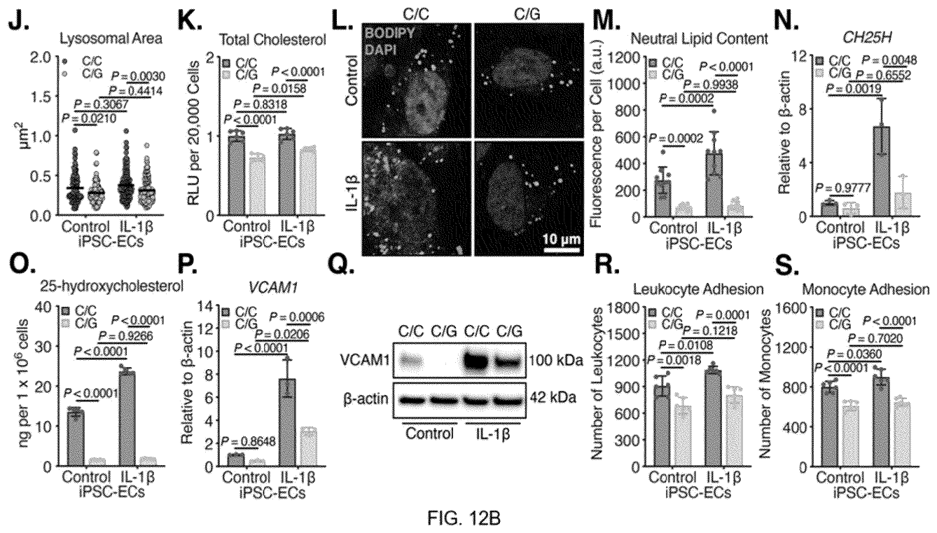

Ncoa7-null mice displayed elevated CH25H expression in pulmonary arterioles, accompanied by elevated plasma levels of 7HOCA and tetrol species (FIG. 14 (B,C,G) and FIG. 13 (F-I)). These findings corresponded with the oxysterol and bile acid plasma signatures associated with PAH severity in humans and our studies of cultured PAECs. The elevation of 7HOCA in Ncoa7-deficient mice resulted in immunoactivation of the endothelium as noted by enhanced VCAM1 expression and CD11b⁺ monocyte infiltration (FIG. 14 (B-E)). Moreover, Ncoa7-null mice displayed increased pulmonary arteriole muscularization (FIG. 14 (F)), accompanied by worsened hemodynamic manifestations of PAH with increased right ventricular systolic pressure (RVSP) and Fulton index—a measure of right ventricular remodeling (FIG. 12A (B,I)).

Finally, to assess the pathogenicity of 7HOCA directly, we utilized a chronically hypoxic mouse model with serial, orotracheal deliveries of either normal saline or 7HOCA (FIG. 14 (J)) and confirmed via echocardiography no alterations in left ventricular function (FIG. 13 (J-N)). As expected, the orotracheal delivery of 7HOCA upregulated immunoactivation of the endothelium, as noted by enhanced VCAM1 expression and CD11b⁺ monocyte infiltration into the vascular bed (FIG. 14 (K-M)). Consistent with the genetic knockout of Ncoa7, 7HOCA worsened PAH, as reflected by increased pulmonary arteriolar remodeling and increased RVSP (FIG. 12B (N,P)). Taken together, our data demonstrated that either a genetic loss of Ncoa7 or direct delivery of the proinflammatory sterol 7HOCA were sufficient to promote PH in vivo.

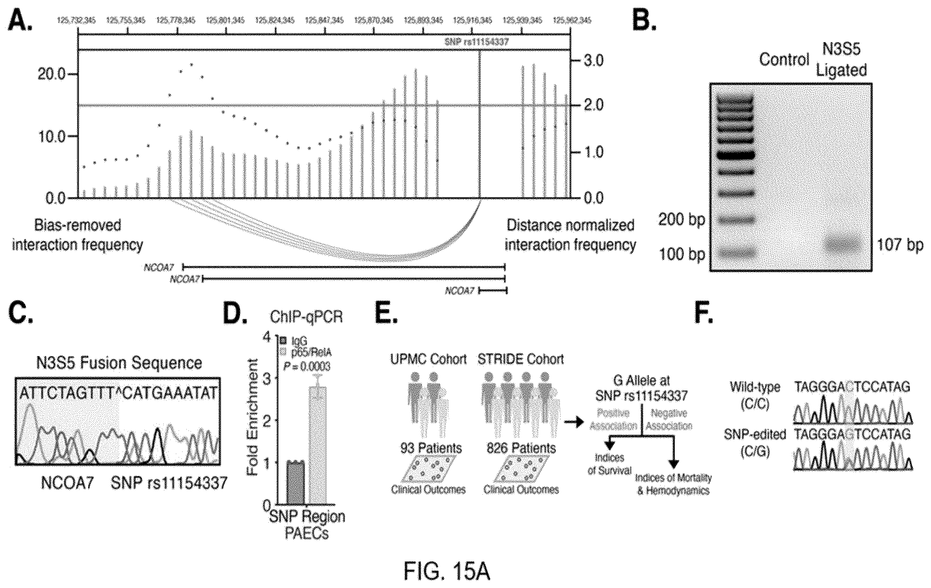

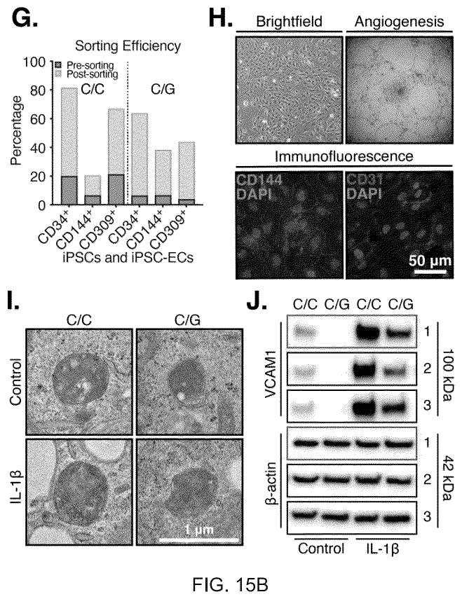

Intronic SNP rs11154337 controls ReIA/p65 binding to the non-canonical promoter of NCOA7 and is associated with PAH disease severity and mortality: These data thus far define NCOA7 as a homeostatic brake in proinflammatory conditions that preserves lysosomal activity and sterol trafficking to attenuate EC immunoactivation in PAH. Based on the role of single nucleotide polymorphisms (SNPs) in regulating gene promoter activity, it was sought to determine if pathogenic NCOA7 deficiencies could result from genetic, SNP-dependent control of NCOA7 expression and its downstream function. First, annotated SNPs were surveyed based on their proximity to the canonical and non-canonical promoters and high levels of epigenetic marks indicating elevated transcriptional activity. In doing so, we found a candidate SNP—rs11154337—located near an intronic region proximal to the non-canonical promoter of NCOA7 and carrying a substantial burden of histone modifications (W. J. Kent et al., The human genome browser at UCSC. Genome Res 12, 996-1006 (2002). Second, given the tandem regulation of both short and long isoforms of NCOA7 in PAH (FIG. 4), it was sought to discern any potential regulatory function of this SNP via positional backfolding onto the canonical promoter. To do so, publicly available high-throughput chromatin conformation capture (3C) was utilized on human umbilical vein endothelial cells (GEO IDs GSM3438650 and GSM3438651) from the 3D-genome Interaction Viewer & database (3 DIV)(D. Yang etal., 3 DIV: A 3D-genome Interaction Viewer and database. Nucleic Acids Res 46, D52-D57 (2018)). Such maps revealed that SNP rs11154337 carries long-range interactions greater than 120 kilobases with the canonical promoter region of NCOA7 (FIG. 15A (A)). To establish this interaction, we performed 3C on human PAECs and found that the 3′ end of the restriction enzyme-digested DNA segment containing the NCOA7 transcription start site (N3) ligates to the 5′ end of the digested segment containing SNP rs11154337 (S5) to produce a nucleotide fusion PCR product confirmed by sequencing (N3S5) (FIG. 15A (B,C)). Furthermore, ChIP against RelA/p65 revealed a significant enrichment for the SNP containing region, denoting the presence of a p65 protein-SNP complex (FIG. 15A (D)).

Given SNP rs11154337 control of NCOA7 gene architecture coupled with the findings that NCOA7 controls the oxysterol signature associated with PAH severity and mortality, it was sought to determine if this SNP was also associated with disease severity and mortality using two independent PAH cohorts. (FIG. 12A (A-C)). First, a single-center PAH cohort of European-descent subjects from the University of Pittsburgh Medical Center (UPMC, N=93) was analyzed. It was found that the G allele was associated with a significant improvement of six-minute walk distance (P=0.0130; β=66.90, 95% Cl [14.45-119.36]), FIG. 12A (A)). Importantly, after adjusting for age, sex, and vasodilator use, survival was significantly increased in patients who carried the homozygous G alleles (P=0.0250; hazard ratio=0.44, 95% Cl [0.21-0.90], FIG. 12A (B)). In a second, multicenter PAH cohort of European-descent from the Sitaxsentan To Relieve Impaired Exercise (STRIDE) trial comprising 45 United States and Canadian pulmonary hypertension centers (STRIDE, N=63 (R. L. Benza etaL., Endothelin-1 Pathway Polymorphisms and Outcomes in Pulmonary Arterial Hypertension. Am J Respir Crit Care Med 192, 1345-1354 (2015)), it was validated that the presence of the G allele confers survival benefit (P=0.0002, hazard ratio=0.49, 95% Cl [0.34-0.71], FIG. 12B (C)). Thus, analysis of genomic, metabolomic, and clinical datasets across cohorts of PAH patients suggested the presence of interconnected activities between NCOA7 and SNP rs11154337 with glucuronidated oxysterols and clinical outcomes of PAH.

SNP rs11154337 modulates NCOA7 and its downstream pathogenic functions: Using this concept—and guided by the negative association of the G allele of the NCOA7 intronic SNP rs11154337 to both the oxysterol signature predictive of mortality and clinical indices of PAH—it was sought to determine if this SNP controls NCOA7 expression, lysosomal activity, and the production of oxysterol and bile acid metabolites to modulate EC behavior. To study the cellular and biological activity of SNP rs11154337 embedded near the non-canonical NCOA7 promoter, a set of genetically matched, isogenic inducible pluripotent stem cell (iPSC) lines were generated with the allelic variants of SNP rs11154337 via CRISPR-Cas9-gene editing (C/C versus C/G genotypes, FIG. 12A (D) and FIG. 15A (F)). The iPSCs were then differentiated into ECs (iPSC-ECs) and purified through a vascular endothelial cadherin (VE-Cadherin; also known as CD144)-based magnetic separation (M. Gu, Efficient Differentiation of Human Pluripotent Stem Cells to Endothelial Cells. Curr Protoc Hum Genet, e64 (2018)). Purified iPSC-ECs exhibited marked enrichment of the EC markers CD34, CD144, and CD309, and immunofluorescent staining of iPSC-ECs against CD144 and CD31 revealed a patterning consistent with the endothelium (FIG. 15B (G,H)). Moreover, iPSC-ECs displayed angiogenic potential, as noted by vessel formation in growth factor-depleted Matrigel (FIG. 15B (H)).

C/G iPSC-ECs displayed higher expression of both short and long NCOA7 isoforms when compared to the C/C line, confirming that the G allele increases NCOA7 transcription (FIG. 12A (E,F)). Providing a putative explanation for the long-range regulation of SNP rs11154337 and the canonical promoter of NCOA7, prior chromatin capture data demonstrated a SNP rs11154337 interaction with the canonical promoter in human umbilical vein endothelial cells (FIG. 15A (A-D)). Consistent with the observed differential in NCOA7 expression and our prior findings with NCOA7 knockdown, lysosomal activity, sterol homeostasis, and immunoactivation in iPSC-ECs were allele-dependent. iPSC-ECs carrying the G allele—and thus higher NCOA7 expression—displayed a concomitant increase in its binding partner ATP6V1B2 and subsequently lower lysosomal pH, as demonstrated by attenuated cleavage of SiR-Lysosome (FIG. 12A (G-I)). Moreover, the presence of the G allele prevented lysosomal hypertrophy in comparison to the iPSC-EC line homozygous for the C allele, indicating maintenance of proper lysosomal acidification and resultant sterol homeostasis (FIG. 12B (J) and FIG. 15B (I,)).

Similar to RNAi against NCOA7, less NCOA7 expression in the homozygous C allele line resulted in higher sterol content and thus drove higher expression of CH25H and its downstream metabolite 25HC (FIG. 12B (K-O))—the enzyme responsible for the generation of downstream oxidized species like 7HOCA. With greater production of 7HOCA, the homozygous C allele iPSC-line displayed elevated immunoactivation of the endothelium, reflected by enhanced VCAM1 expression and immune-cell adhesion (FIG. 12B (P-S)). Thus, consistent with the association of the G allele of SNP rs11154337 as protective against oxysterol production and PAH severity, the G allele increased NCOA7 expression, its downstream modulation of lysosomal acidification, oxysterol generation, and consequent EC immunoactivation.

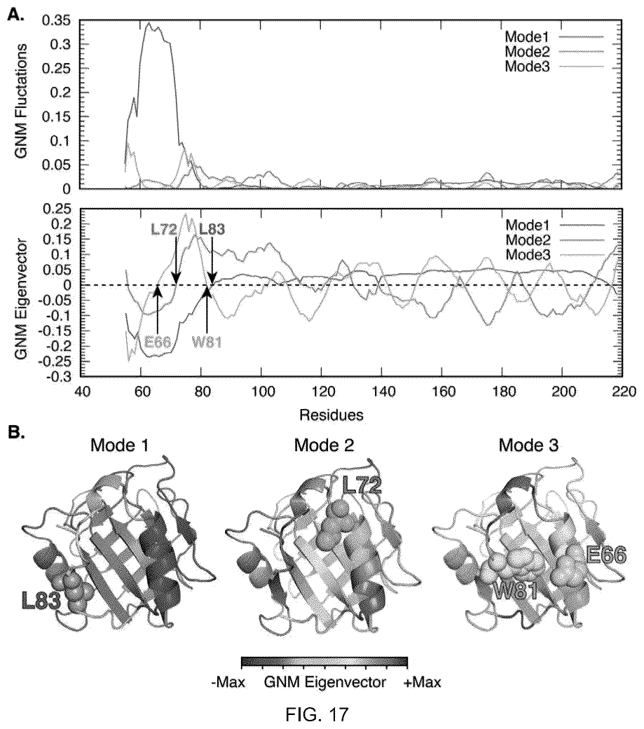

Structural modeling and molecular simulations identify a novel therapeutic activator of NCOA7: Toward identifying a small molecule activator of NCOA7, we performed structure-based computations composed of three parts: druggability simulations, pharmacophore modeling, and virtual screening (FIG. 16A (A-C). Druggability simulations were carried out using the model structure of NCOA7 in the presence of explicit water and probe molecules representative of drug-like fragments. We used the probe molecules acetamide, acetate, benzene, imidazole, isobutane, isopropanol, and isopropylamine in six independent runs of 40 ns each. A molecular pocket was distinguished in three of the runs through its high affinity to bind the probe molecules (FIG. 16A (A), cyan spheres). This site also demonstrates hinge residues from the Gaussian Network Model (GNM) analysis of NCOA7 (FIG. 17 (A)). The hinge residues at or near the binding pocket are L83 (mode 1), L72 (mode 2), and E66 and W81 (mode 3) (FIG. 17 (B)). Notably, hinge sites have been shown in previous work to have a critical role in mediating the functional dynamics of proteins, and, as such, are used as target sites for binding small molecule modulators of protein function. For this reason, the identified molecular pocket was selected for further analysis using pharmacophore modeling boosted by both druggability simulations and GNM analysis.

Using our tool Pharmmaker, we performed pharmacophore modeling, and subsequently selected the high affinity residues (i.e., N62, E66, P80, and W81; FIG. 16A (B)) at the identified molecular pocket (J. Y. Lee, et al., Pharmmaker: Pharmacophore modeling and hit identification based on druggability simulations. Protein Sci 29, 76-86 (2020)). The interactions of these residues with the probe molecules were ranked based on their occurrence frequency during simulations. Notably, among the probe molecules, two benzenes demonstrated a high propensity to interact with N62, P80, and W81, while one isopropylamine interacted with E66 (FIG. 16A (B), benzenes in black and isopropylamine in blue). Molecular dynamics (MD) snapshots that simultaneously displayed multiple frequently observed (i.e., entropically favored) interactions were used to construct the pharmacophore model, which was composed of one hydrogen bond donor, three hydrophobic rings, and two aromatic rings (FIG. 16A (C)).

The pharmacophore model was then screened against the ZINC (T. Sterling, J. J. Irwin, ZINC 15—Ligand Discovery for Everyone. J Chem Inf Model 55, 2324-2337 (2015)) and MolPort small molecule libraries via the Pharmit (T. Sterling, J. J. Irwin, ZINC 15—Ligand Discovery for Everyone. J Chem Inf Model 55, 2324-2337 (2015)) server to obtain an ensemble of compounds. The top scoring compounds were selected as hits for further experimental validation. The compound MolPort-004-267-958 (herein called 958) yielded preliminary data, suggesting it as an activator of NCOA7 relative to the other predicted compounds. We then sought to further examine the binding behavior of 958 through MD simulations.

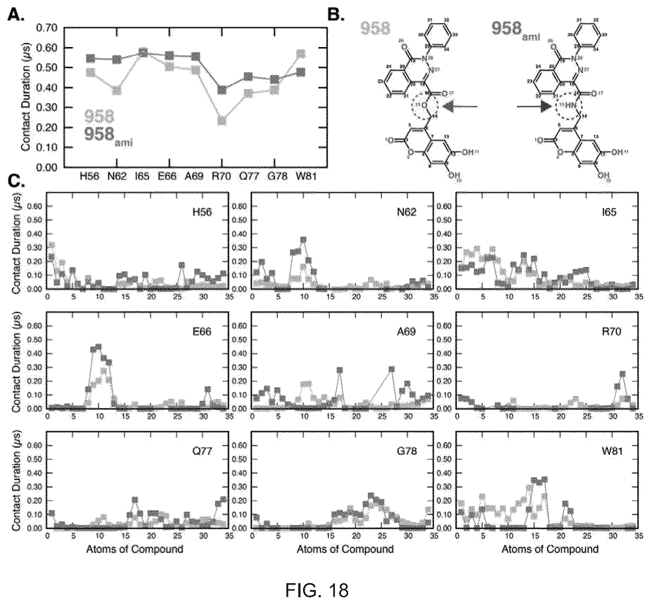

Refinement of the activator 958 led to its analogue 958ₐₘᵢ that has stronger binding affinity: To create a molecule that more avidly binds to the molecular pocket of NCOA7, we performed MD simulations. As such, all-atom 0.6 μs MD simulations (or three independent, 0.2 μs runs) of 958 were performed to characterize the most critical functional groups and interactions. Based on these simulations, we designed an analogue of 958, where the O₁₅ atom of the ester functional group is replaced with N₁₅-H to create an amide functional group with the resultant compound herein called 958ₐₘᵢ (FIG. 16A (D,E)). Subsequent MD simulations using 958ₐₘᵢ further clarified its enhanced activity. To compare the parental and analogue structures, we selected nine residues with high interaction affinities to the compounds: H56, N62, I65, E66, A69, R70, Q77, G78, and W81. Overall contact duration for these residues was greater than 0.35 μs out of 0.6 μs with 958 or 958ₐₘᵢ. (FIG. 18 (A-C)). Strong interactions, defined as a contact duration greater than 0.3 μs, were formed by H56-O₁ in 958 versus N62-O₁₀, E66-O₁₀/O₁₁, and W81-N₅/O₇ in 958ₐₘᵢ (FIG. 16A (D); orange solid lines). Notably, atoms 16 through 34 do not undergo any significant interactions with the NCOA7 binding pocket; however, the substitution of O₁₅ with N₁₅—H resulted in multiple strong interactions with many residues within the pocket. Additionally, the substitution with N₁₅ exhibited strong interactions with 165 and W81, caused new interactions of atoms 16 through 34 with A69, R70, and Q77, and strengthened the C₂₃/C₂₄-G78 and O₁₀/O₁₁-N62/E66 interactions. Lastly, using hydrogen bond analysis with a cutoff distance of 3.0 Å between donor and acceptor atoms with a 160° angle, E66 was identified with a significantly higher propensity to form hydrogen bonds with 958ₐₘᵢ.

Using PRODIGY-LIG (T. Sterling, J. J. Irwin, ZINC 15—Ligand Discovery for Everyone. J Chem Inf Model 55, 2324-2337 (2015)), calculated binding affinities revealed that 958ₐₘᵢ was more stably bound to NCOA7 than the parental compound 958 (FIG. 19 (A)). The corresponding binding pose at 100 to 200 ns had a binding affinity of −8.16±0.16 kcal/mol (FIG. 16A (E), run 1). 958ₐₘᵢ also had two additional stable poses: one where W81 lost its interaction with N₁₅ (FIG. 19 (B), run 2, −7.71±0.16 kcal/mol) and the other where the compound rotated upside down within the pocket (FIG. 19 (B), run 3, −8.83±0.19 kcal/mol).

Administration of the NCOA7 activator 958ₐₘᵢ reverses disease in a PAH model: To assess the downstream molecular functions of NCOA7 activation with 958ₐₘᵢ, we performed a proximity ligation assay to assess ATP6V1B2-NCOA7 interactions. As expected, application of 958ₐₘᵢ significantly induced the number of amplifications per cell, suggesting a molecular enhancement at the level of lysosome (FIG. 16B (F,G)). The maintenance of lysosomal acidification with 958ₐₘᵢ similarly prevented induction of CH25H under IL-1β, which further corresponded to decreased EC immunoactivation as noted by VCAM1 expression and immune cell adhesion to a monolayer (FIG. 16B (H-L)).

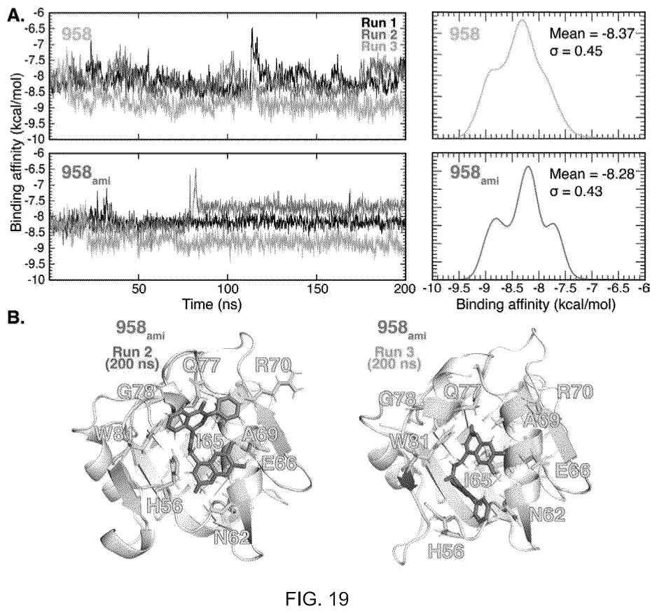

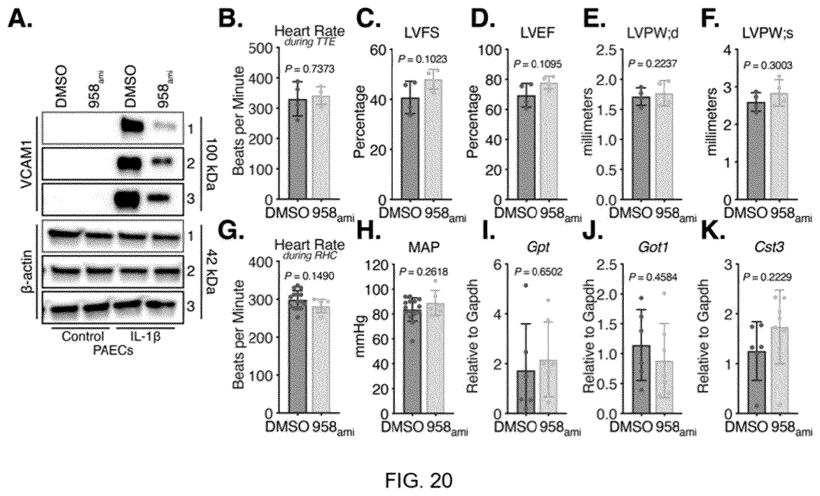

Next, we sought to confirm if 958ₐₘᵢ would protect against endothelial immunoactivation in the proinflammatory monocrotaline PAH rat model. Rats were injected intraperitoneally with DMSO or 958ₐₘᵢ (7.5 mg/kg) for 10 days post-monocrotaline loading (FIG. 16B (M)). Rodents treated with 958ₐₘᵢ neither had appreciable hepatic or renal toxicity nor alterations in left ventricular function as compared to vehicle controls (FIG. 20 (B-K)). Rats treated with the NCOA7 activator 958ₐₘᵢ exhibited decreased CH25H expression with a corresponding attenuation in VCAM1 expression at the endothelium and CD11b⁺ monocyte infiltration at the pulmonary vessel (FIGS. 16B and 16C (N-Q)). As a result, pulmonary vessels demonstrated decreased muscularization, which corresponded to a significant reduction in both right ventricular hypertrophy and RVSP (FIG. 16C (R-T)). Overall, these data identified 958ₐₘᵢ as a novel therapeutic agent that reverses PAH pathogenesis with potential implications related to diseases of immune dysregulation.

By harnessing large-scale, multidimensional genomic and metabolomic analytics with concomitant mechanistic experimentation, it was found that NCOA7 regulates lysosomal activity and EC sterol metabolism to function as a homeostatic brake and prevents oxysterol-induced inflammation, EC dysfunction, and PAH. Most notably, the presence of the G allele at SNP rs11154337 results in enhanced NCOA7 expression, thereby reducing inflammation in PAH and establishing mechanistic proof of the underlying genetic association between SNP rs11154337 and PAH mortality and the metabolomic association between the oxysterol signature and PAH severity. Ultimately, this work establishes a new paradigm that links fundamental lysosomal biology and oxysterol metabolism to EC behavior with broad implications in the development of molecular diagnostics and therapeutics in PAH, as well as other inflammatory vascular disorders.

The identification of NCOA7 as a primary controller of PAH has broad implications for human disease. The role of NCOA7 in immunomodulation described herein is believed to point to much broader actions of NCOA7 isoforms and related proteins. While recent studies have reported unique activity of the short-length isoform of NCOA7, the findings described herein point toward an additive or synergistic behavior of both isoforms in modulating oxysterol-mediated EC inflammation. The NCOA7 isoforms carry a Tre2/Bub2/Cdc16 (TBC), lysin motif (LysM), domain catalytic (TLDc) domain. All TLDc-containing proteins physically interact with V-ATPases, thereby defining a new class of V-ATPase regulatory proteins.

At the cellular level, this work highlights a broad lysosomal role in EC function and PAH. Prior clinical observations have suggested a relationship between rare, recessive, loss-of-function lysosomal storage disorders and pulmonary vascular diseases. For example, human mutations in various V-ATPase subunits (e.g., ATP6V1A and ATP6V1E1) can present with pulmonary arterial stenosis or hypoplasia and right ventricular hypertrophy (T. Van Damme et al., Mutations in ATP6V1E1 or ATP6V1A Cause Autosomal-Recessive Cutis Laxa. Am J Hum Genet 100, 216-227 (2017)). PAH has been seen in mucolipidosis—a disease driven by dysfunctional lysosomal enzyme processing. High pulmonary arterial pressures were reported in patients with Gaucher's disease—a condition resulting from deficiency of lysosomal β-glucosidase (FIG. 6B (F,G) and carrying a known association to Group V PH. Moreover, Niemann-Pick disease and Fabry disease manifest with severe pulmonary dysfunction, which often coexist with Group III PH. Supported by these rare genetic diseases, the association of the homozygous C/C genotype to worsened survival in PAH offers broader and more definitive proof of a causative link between lysosomal dysfunction and PAH. In fact, guided by the C mean allele frequency (˜0.48 to 0.52) of SNP rs1115447 in the global population, approximately a quarter of PAH patients harboring the C/C genotype would be expected to suffer from worsened mortality. Furthermore, based on the principle of synergistic heterozygosity previously reported in the context of BMPR2 mutations in familial PAH, it remains to be seen whether worsened PAH or other lysosomal storage disorders may manifest to an even greater extent in monoallelic carriers of known familial PAH mutations or lysosomal enzyme mutations if accompanied by the SNP rs1115447 C/C genotype.

In summary, via multi-dimensional analyses of genomic and metabolomic datasets in combination with in vitro and in vivo mechanistic validation, we defined the fundamental and SNP-dependent role of NCOA7 and its control of lysosomal activity and sterol homeostasis to temper inflammation, EC dysfunction, and PAH. In the computational modeling of the molecular pocket of NCOA7, the compound 958ₐₘᵢ, and derivatives thereof, as described herein, were developed. In vitro and in vivo studies reveal the prevention of oxysterol-mediated endothelial immunoactivation and reversal of disease in the PAH monocrotaline rat by compound 958ₐₘᵢ, and therapies like 958ₐₘᵢ represent a compelling complement to existing vasodilator therapies and other disease-modifying agents in development, which mainly target cellular proliferative and survival pathways.

### Example 2: Viral In Vitro Assays

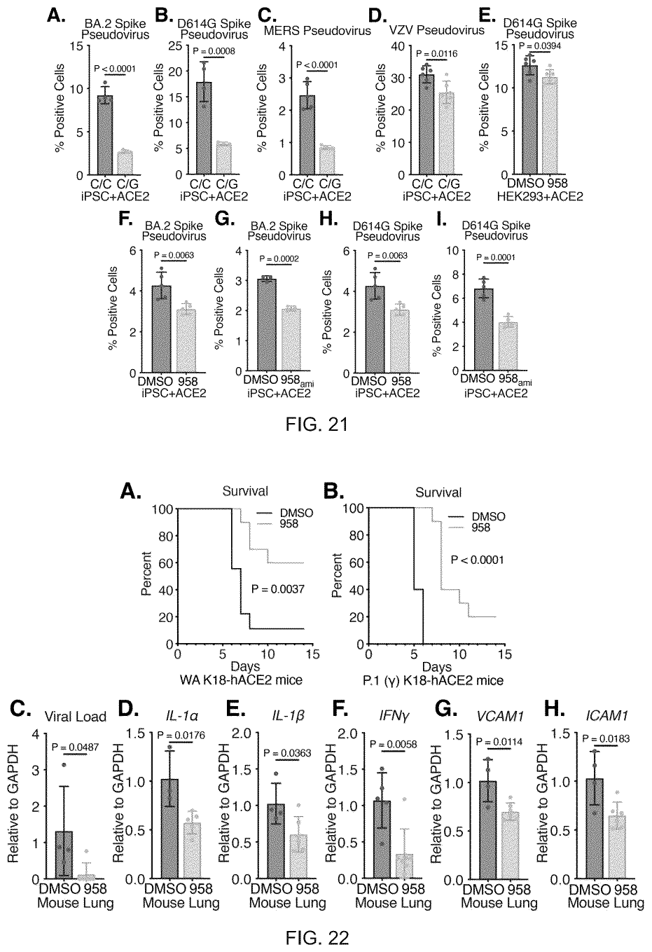

SNP rs11154337 and 958 modulate the entry of various pseudotyped envelope viruses. As shown in FIG. 21, iPSCs transfected with human ACE2 receptor demonstrate decreased entry of multiple pseudotyped coronaviruses and a herpesvirus with the presence of the G allele at SNP rs11154337 iPSCs transfected with human ACE2 receptor demonstrate decreased entry of multiple pseudotyped coronaviruses and a herpesvirus with the presence of the G allele at SNP rs11154337, which confers increased NCOA7 expression. Further, application of the NCOA7 activator 958 enhances the activity of NCOA7 to prevent pseudotyped SARS-CoV-2 infection (D614G Spike) in HEK293 cells transfected with human ACE2 receptor. 958 parental (2 μM) and 958ₐₘᵢ (5 μM) decrease BA.2 spike pseudotyped virus infection in iPSCs transfected with the human ACE2 receptor. Also, 958 parental (2 μM) and 958ₐₘᵢ (5 μM) decreased D614G spike pseudotyped virus infection in iPSCs transfected with the human ACE2 receptor.

### Example 3: SARS-CoV2 In Vivo Assay

Referring to FIG. 22, Coronavirus-infected human ACE2 transgenic mice treated with the NCOA7 activator 958 have decreased lung inflammation and mortality. Human ACE2 transgenic mice infected with two different coronavirus strains have significantly improved mortality when treated with 958. Further, mouse lung tissue demonstrates decreased viral load and attenuation of various proinflammatory markers (IL-1α, IL-1β, IFN-γ, VCAM1, and ICAM1) when treated with 958 via RT-qPCR.

### Example 4: In Vivo Reduction of Lung Inflammation in Bacterial Infection

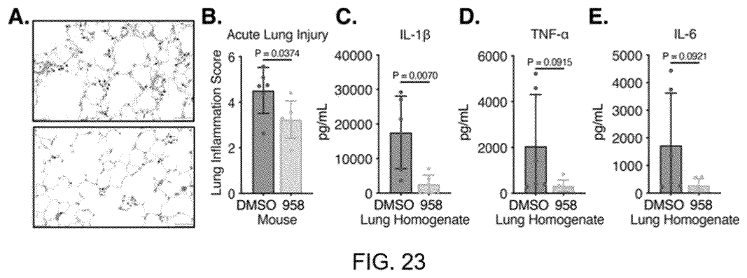

Mice were infected with Klebsiella pneumoniae. Referring to FIG. 23, those mice demonstrated improvement of acute lung injury when treated with the NCOA7 activator 958. (FIG. 23 (A, B)) Representative images were prepared of lung tissue sections stained with H&E after 48 hours of intratracheal infection with K. pneumoniae in mice treated with DMSO compared to 958 (5 mg/kg IP dosing daily for 3 days). Mice treated with 958 have decreased inflammation (FIG. 23 (C to E)) as shown by ELISA on lung homogenate of proinflammatory cytokines IL-1β, TNF-α, and IL-6.

### Example 5—Stroke

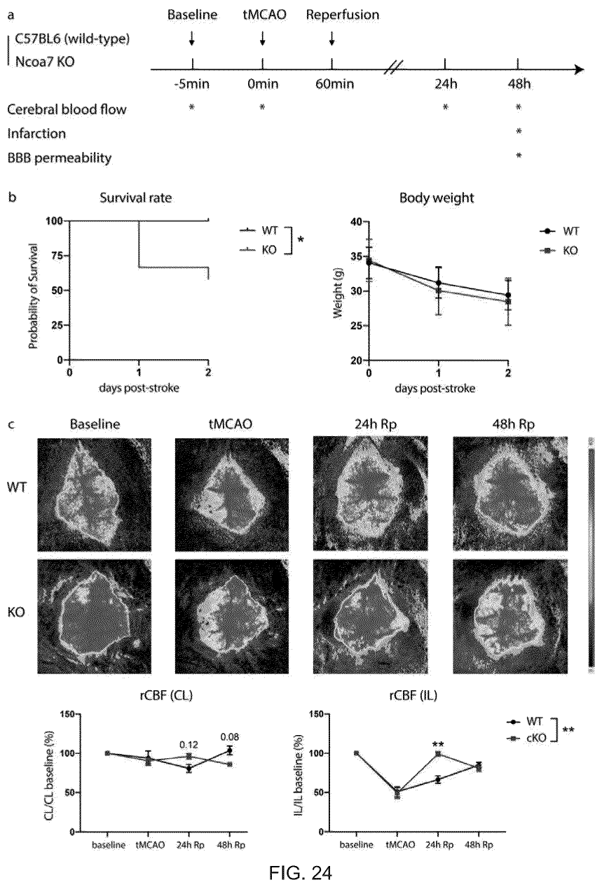

Loss of Ncoa7 worsens survival in a mouse model of ischemic stroke. Referring to FIG. 24, wildtype versus Ncoa7-deficient mice were subjected to an experimental model of ischemic stroke. Mice received either a sham surgery or transient middle cerebral artery occlusion (tMCAO) for 60 minutes before restoration of cerebral blood flow. Mice were then collected at 24 and 48 hours for further study. Ncoa7 knockout mice were seen to have significantly decreased survival at 24 and 48 hours post-tMCAO compared to wildtype controls. There was no appreciable change in body weight at 24 or 48 hours post-tMCAO. Using Laser Doppler Flowmetry, regional cerebral blood flow (rCBF) was quantified in the contralateral (CL) and ipsilateral (IL) cerebral cortices of mice. Mice deficient for Ncoa7 had increased rCBF 24 hours post-tMCAO in the IL cerebral cortex compared to wildtype controls.

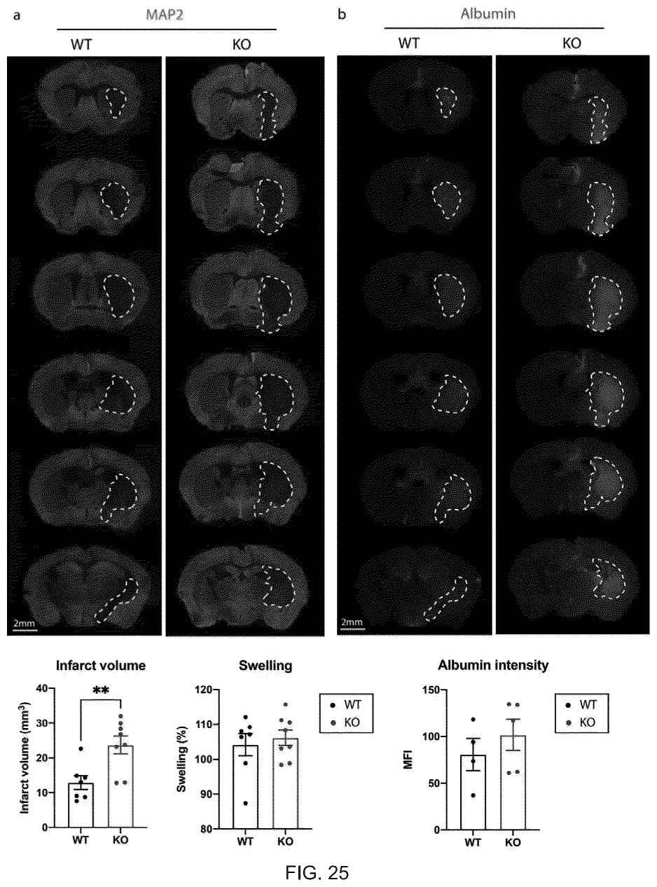

Loss of NCOA7 increases infarct volume and capillary leak in mice post-ischemic stroke. As shown in FIG. 25, mouse brain tissue was sectioned in 30 microns before immunofluorescent staining. Brain sections were stained with the neuronal marker microtubule-associated protein 2 (MAP2) to assess for infarct volume post-tMCAO. In mice deficient for NCOA7, there is significant increase in infarct volume after ischemic stroke, indicating greater neuronal cell death. There was no appreciable change in tissue swelling compared to wildtype controls. To assess capillary leakage, brain sections were stained for the plasma protein albumin. Notably, mice deficient for NCOA7 had a trend toward greater leakage of albumin into brain tissue post-tMCAO, indicating greater damage to the brain microvasculature as compared to wildtype controls.

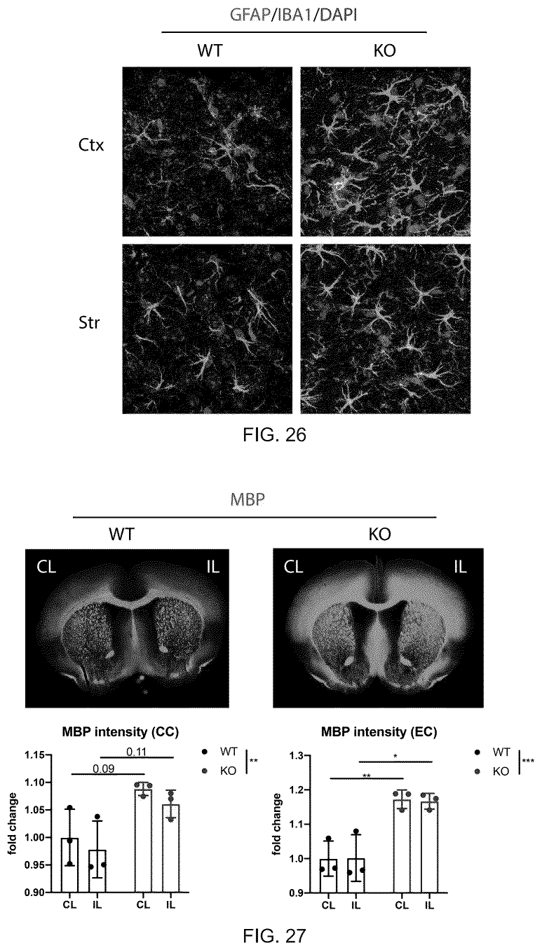

Loss of NCOA7 results in worsened neuroinflammation after ischemic stroke. As shown in FIG. 26, mouse brain sections were stained using an immunofluorescent protocol. Brains were stained with the astrocytic marker glial fibrillary acidic protein (GFAP), the microglial marker ionized calcium binding adaptor molecule 1 (IBA1), and a nuclear marker (DAPI). Images were obtained using confocal microscopy in both the cortex (Ctx) and striatum (Str) of wildtype and Ncoa7-knockout mice after tMCAO. Mice deficient for NCOA7 have markedly worse neuroinflammation as noted by swelling of the astrocytic processes in both cortical and striatal tissue. In addition, microglia are hypertrophic and elongated in knockout mice as compared to wildtype controls. These data indicate substantial reactive gliosis and neuroinflammation in brains deficient for NCOA7 post-tMCAO.

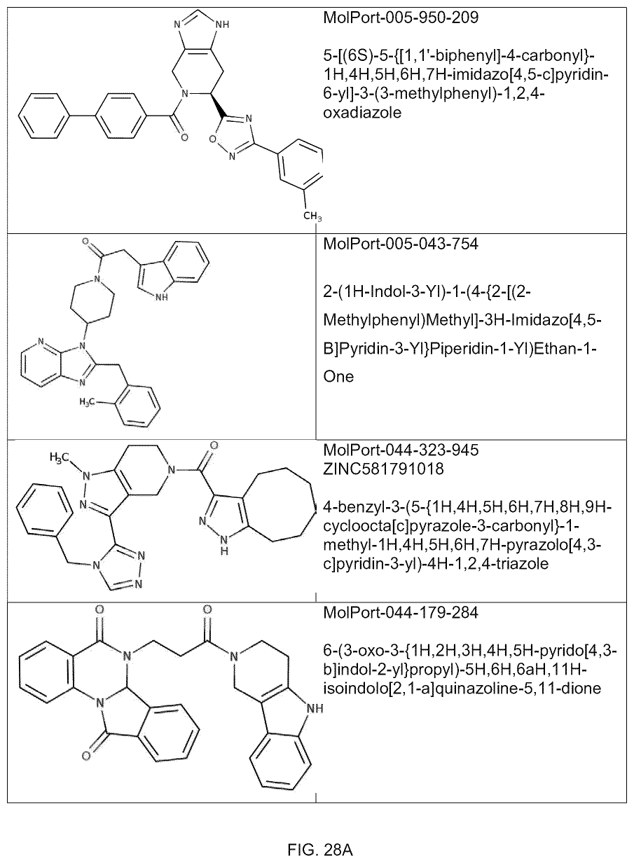

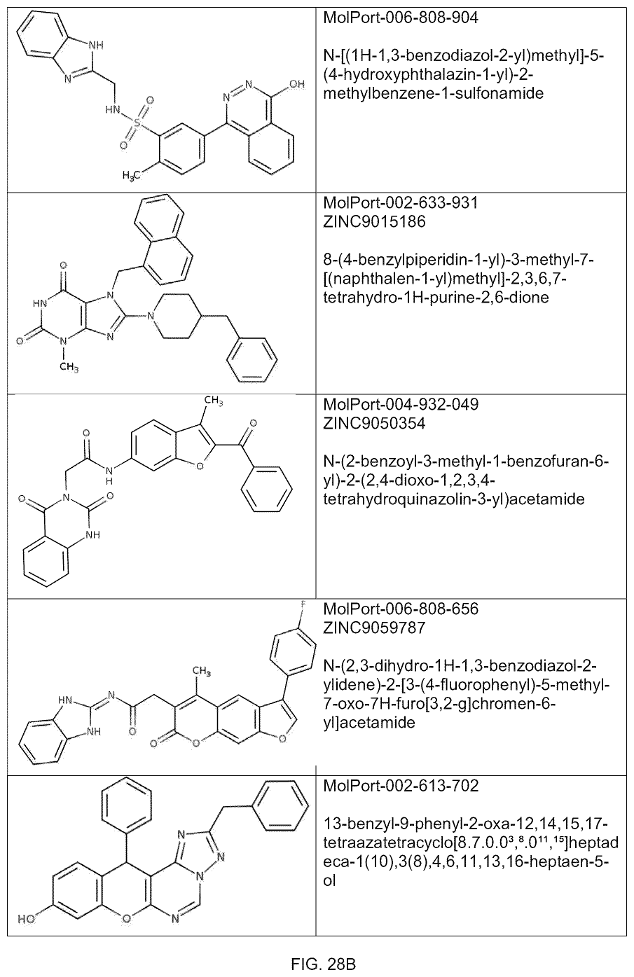

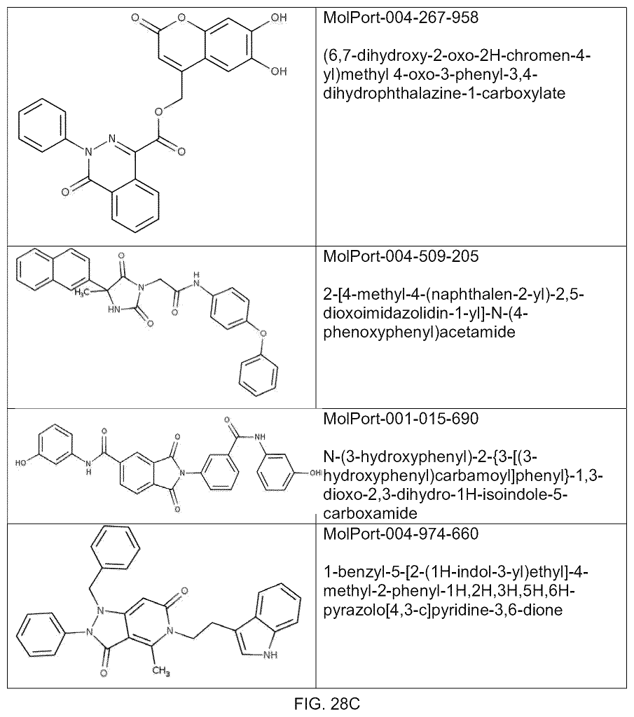

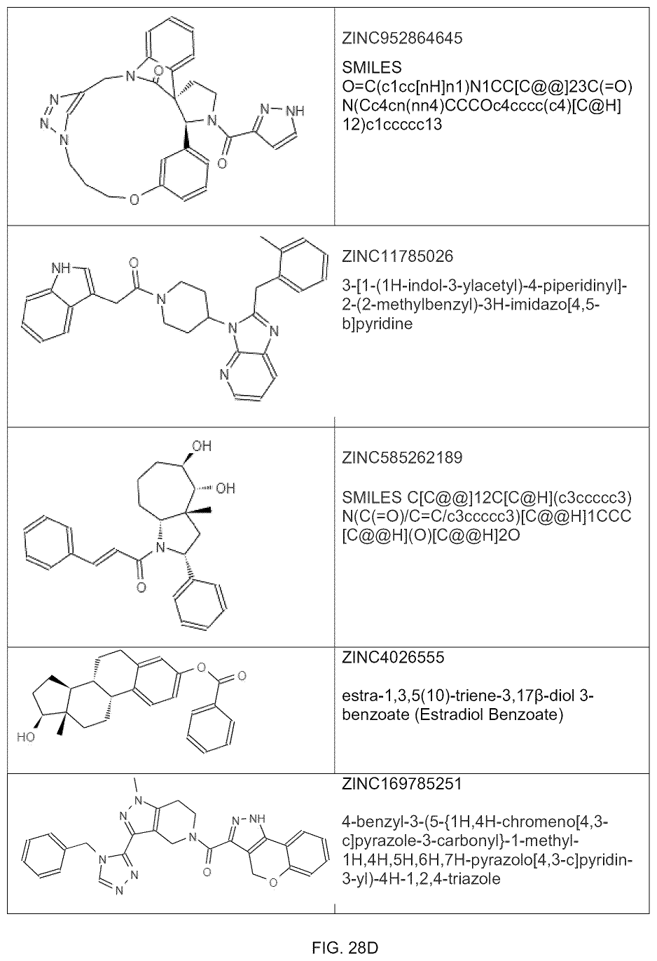

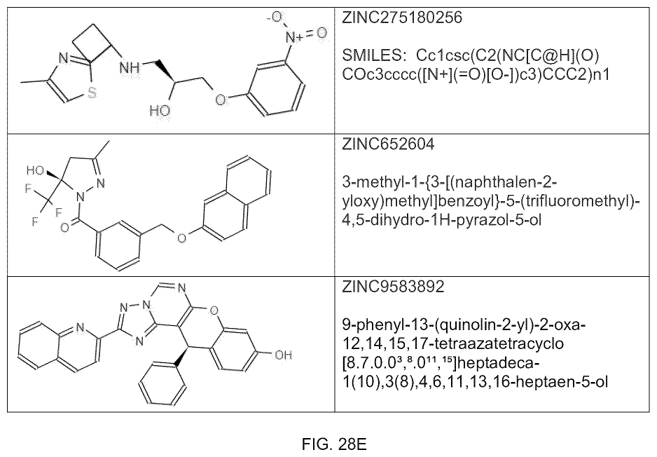

Loss of NCOA7 results in significant hypermyelination in a mouse model of ischemic stroke. Referring to FIG. 27, brain sections were stained with myelin basic protein (MBP) using an immunofluorescent protocol. NCOA7-deficient mice had significantly enhanced myelination globally, especially at the levels of the corpus callosum (CC) and the external capsule (EC). Notably, there was no appreciable difference in comparison of contralateral (CL) versus ipsilateral (IL) hemispheres post-tMCAO.

The present invention has been described with reference to certain exemplary embodiments. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of treating inflammation in a patient, such as a human patient, comprising:
administering to the patient an amount of a compound, having the structure: 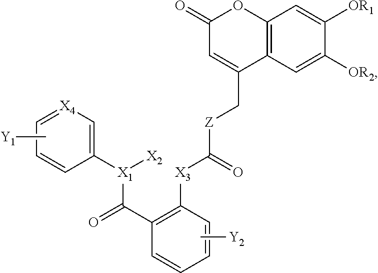
wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl, —NHR₆ where R₆ is —H or —C₁₋₃ alkyl, —NHC(O)—R₇ where R₇ is —H or —C₁₋₃ alkyl, —OR₈ where R₈ is —H or —C₁₋₃ alkyl, —OC(O)—R₉ where R₉ is —H or —C₁₋₃ alkyl, —C(O)—R₁₀ where R₁₀ is —H or —C₁₋₃ alkyl, or —C(O)—R₁₁-R₁₂ where R₁₁ is O or NH and R₁₂ is —H or —C₁₋₃ alkyl; or
one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, or; ZINC9583892,
or a pharmaceutically acceptable salt thereof, in an amount effective to reduce inflammation in a patient.

2. The method of claim 1, wherein the inflammation is cardiovascular inflammation or vascular inflammation, or is associated with a disease such as pulmonary hypertension, restenosis, essential hypertension, atherosclerosis, viral infection, bacterial infection, fungal infection, parasite infection, COVID (coronavirus disease), ARDS (acute respiratory distress syndrome), acute lung injury, stroke, neurodegeneration, cancer, an autoimmune disease, or a disease innate and acquired immunity.

3. The method of claim 1, wherein the patient has vascular inflammation.

4. The method of claim 1, wherein the inflammation is associated with a viral or bacterial infection.

5. The method of claim 2, wherein the inflammation is associated with a coronavirus or herpesvirus infection in the patient, optionally a severe acute respiratory syndrome from a coronavirus infection in the patient.

6. The method of claim 2, wherein the inflammation is associated with a bacterial infection in the patient; such as a Klebsiella pneumoniae infection in the patient, and optionally further comprising reducing or preventing lung damage in the patient.

7. (canceled)

8. The method of claim 1, wherein the inflammation is associated with one of more of: pulmonary arterial hypertension (PAH), pulmonary hypertension due to left heart disease, pulmonary hypertension due to lung disease, pulmonary hypertension due to chronic blood clots in the lungs, and pulmonary hypertension due to unknown causes.

9. (canceled)

10. The method of claim 1, wherein Z is NH, R₁ and R₂ are, independently, Me or H, or R₁ and R₂ are H.

11-12. (canceled)

13. The method of claim 1, wherein the compound is compound 958 (MolPort-004-267-958), or a pharmaceutically acceptable salt thereof, or compound 958ₐₘᵢ, having the exemplary structure: 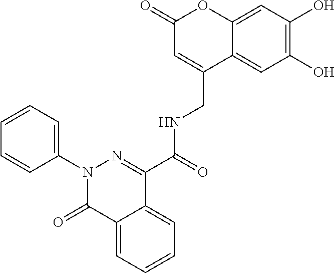 [SMILES: O=C(NCc1cc(=O)oc2cc(O)c(O)cc12)c1nn(-c2ccccc2)c(=O)c2ccccc12]
or a pharmaceutically acceptable salt, thereof.

14. (canceled)

15. The method of claim 1, wherein the patient is administered an amount of the compound, or a pharmaceutically-acceptable salt thereof, effective to reduce inflammation in the patient or to treat pulmonary hypertension in the patient.

16. The method of claim 1, comprising administering to the patient from 1 μg to 10 g, or from 1 ng to 100 mg/kg of the compound per day, or to a concentration ranging from 1 to 40 μM in a patient's bodily fluid, e.g. blood, serum, plasma, etc.

17. The method of claim 1, wherein the patient is heterozygous or homozygous for C at rs11154337.

18. (canceled)

19. The method of claim 17, further comprising obtaining genetic data for the patient and determining if the patient has one or two alleles for C at rs11154337.

20-23. (canceled)

24. A compound, comprising the structure: 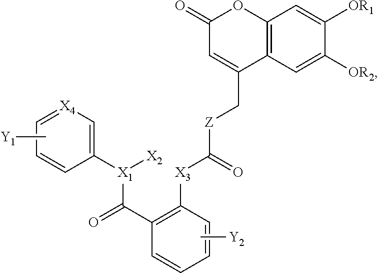
wherein
R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo, or —NO₂; Y₁ is —H, —C₁₋₃ alkyl, halo, —NO₂, —CN, —CF₃, —SO₂R₄ where R₄ is —OH, —C₁₋₃ alkyl, —NHR₅ where R₅ is H or —C₁₋₃ alkyl, —NHR₆ where R₆ is —H or C₁₋₃ alkyl, —NHC(O)—R₇ where R₇ is —H or —C₁₋₃ alkyl, —OR₈ where R₈ is —H or —C₁₋₃ alkyl), —OC(O)—R₉ where R₉ is —H or —C₁₋₃ alkyl), —C(O)—R₁₀ where R₁₀ is —H or —C₁₋₃ alkyl), or —C(O)—R₁₁—R₁₂ where R₁₁ is O or NH and R₁₂ is —H, —C₁₋₃ alkyl), or a pharmaceutically acceptable salt thereof, excluding MolPort-004-267-958.

25. The compound of claim 24, wherein Z is NH, R₁ and R₂ are, independently, Me or H, or R₁ and R₂ are H.

26-27. (canceled)

28. The compound of claim 24, having the structure: 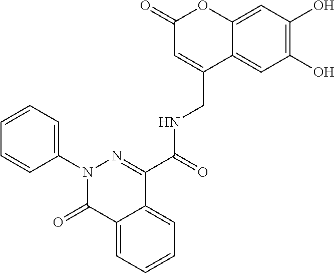 [SMILES: O=C(NCc1cc(=O)oc2cc(O)c(O)cc12)c1nn(-c2ccccc2)c(=O)c2ccccc12]
or a pharmaceutically acceptable salt thereof.

29. A pharmaceutical composition comprising:
a compound either:
having the structure: 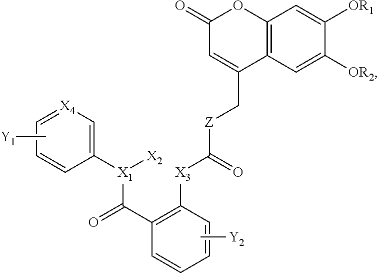
wherein R₁ and R₂ are, independently, —H or —C₁₋₃ alkyl; Z is O or NH; X₁,X₂,X₃ are, independently, N or C; X₄ is ortho, meta or para to X₁ and is N or C; Y₂ is —H, —C₁₋₃ alkyl, halo, or —NO₂; Y₁ is —H, —C₁₋₃ alkyl, halo, —NO₂, —CN, —CF₃, —SO₂R₄ where R₄ is —OH or —C₁₋₃ alkyl, —NHR₅ where R₅ is H or —C₁₋₃ alkyl, —NHR₆ where R₆ is —H or —C₁₋₃ alkyl, —NHC(O)—R₇ where R₇ is —H or —C₁₋₃ alkyl, —OR₈ where R₈ is —H or —C₁₋₃ alkyl, —OC(O)—R₉ where R₉ is —H or —C₁₋₃ alkyl, —C(O)—R₁₀ where R₁₀ is —H or —C₁₋₃ alkyl, or —C(O)—R₁₁-R₁₂ where R₁₁ is O or NH and R₁₂ is —H or —C₁₋₃ alkyl, or a pharmaceutically acceptable salt thereof; or
is chosen from one or more of MolPort-005-950-209; MolPort-005-043-754; MolPort-044-323-945 (ZINC581791018); MolPort-044-179-284; MolPort-006-808-904; MolPort-002-633-931 (ZINC9015186); MolPort-004-932-049 (ZINC9050354); MolPort-006-808-656 (ZINC9059787); MolPort-002-613-702; MolPort-004-267-958; MolPort-004-509-205; MolPort-001-015-690; MolPort-004-974-660; ZINC952864645; ZINC11785026; ZINC585262189; ZINC4026555; ZINC169785251; ZINC275180256; ZINC652604, and ZINC9583892, or a pharmaceutically acceptable salt thereof; and
a pharmaceutically-effective excipient,
wherein the composition comprises an amount of the compound effective to treat or reduce inflammation, cardiovascular inflammation, vascular inflammation (e.g., having vascular endothelium inflammation or a disease having vascular endothelium inflammation as a symptom, such as pulmonary hypertension, restenosis, essential hypertension, atherosclerosis, and stroke), a disease characterized by vascular inflammation, or a disease of innate and acquired immunity, or for treating a coronavirus infection, such as a SARS-CoV-2 infection in a patient, such as a human patient.

30. The composition of claim 29, wherein Z is NH, R₁ and R₂ are, independently, Me or H, or R₁ and R₂ are H.

31-32. (canceled)

33. The composition of claim 29, comprising a compound having the structure: 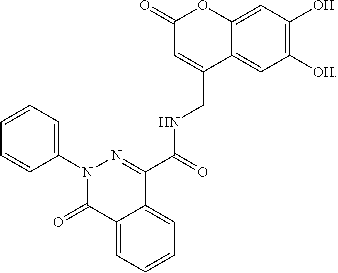 [SMILES: O=C(NCc1cc(=O)oc2cc(O)c(O)cc12)c1nn(-c2ccccc2)c(=O)c2ccccc12]
or a pharmaceutically acceptable salt thereof.

34. The composition of claim 29, comprising MolPort-004-267-958.

35. (canceled)


SEQUENCE LISTING (30 sequences)

SEQ ID NO: 1 - DNA, 201 bp, Homo sapiens
  source 1..201: mol_type = other DNA
  variation 101: note = C or G
tggtaaaagccaggttgaagtggaaaggaagggcatgtgtctagtttatgcctctttatccagcttgatctgtgtcttcaaatatatagcaggatagggantccatagtcatgtccctgaatgggaagacacctcctggccagtatccttgccaaggtaaacactgtcaaatgatcattttcttggtttagaatagcaaat

SEQ ID NO: 2 - RNA, 4025 nt, Homo sapiens
  source 1..4025: mol_type = mRNA
agttgtagctcagcgtggctacaagtaactgtggtgtggaagcagagtagagagaaaacttgttcctcattagagagagagccacacttctcactgctcacaatgagaggccaaagattacccttggacatccagattttctattgtgccagacctgacgaagagccttttgtgaagatcatcactgttgaagaggcaaagcgcaggaagagcacatgcagctactatgaagacgaggacgaagaggtgctgcctgtcctacggccccacagcgcgctcctggagaatatgcacatcgagcagctggcccgacgccttcctgcaagggtgcaagggtatccatggagactggcctatagcacgttagagcacgggaccagcttaaagacgctctaccggaaatcggcatcactagacagtcctgtcctattggtcatcaaagatatggataatcagatttttggagcatatgcaactcatcctttcaagttcagtgaccactattatggcacaggcgaaacttttctctacacattcagccctcattttaaggtctttaagtggagtggagaaaattcatactttatcaatggagacataagttctttagaacttggtggtggagggggacgatttggtttatggctagatgctgatttataccacggacgaagcaactcttgcagcactttcaataatgatattctttccaaaaaggaagacttcatagttcaggatctggaggtgtgggcatttgattgaaattcagactgccttaaaatataacattaaaaagactgggttcgatcagccctcctaaagctggctggaaaaagaagccccagcccagcctgcctcatcccaccccaatgcttcctttctgccatcatctcagagcatgatcacattgcagaaagattctggaaggtccatgtagagggcagacattgaagaaagaaacttaaaatccaggttgttgaaaagactttgtactcccacttcctccaaatccatacagtgaggaatcagagtgtttatagatatatgagttgaatgcaatttttatttttggtaactgtgaaaaataagatactgtggatatatacatgccttgtgtatttatcagcataattttcattaccaaaatgtacagctattatttgccatggaaaaggttagtctcatttagaaaaatcgaaagtgcacagcacttaaagggaatatatgaggtttttttttttttaaaaaaaaacttttatttattatttgtagtatattgtctgaaatgtgtcggcagttttttttcttttaatgtgtcaaatcttgaaatattaaatgtatacattttgtgctatgttttgggaacaaatctgtttgatttatatagttttatattgaattttttttgccctgattgtttagggtgataggtcttaagcagcatatatctatatatctgtatgtgggtatatagagatatatgtgtgtgtgtatgtatatgtacatatacatatatatgagtgtataattctaaatttctaaaaactcattatgaatgttcatcaatttgactattataggccagctttccatttagtcaataaaagcgtacatttttagttacttaccttgaacatattcgtgtgaaaaagaatacatcatttctcacagtcttaagttgatatttatagaaatgaatacctttgtgaacctagacttagaacaaatcctgcttttgaaaaaaaatgttttgcttcttacaaaatcatttgtgttaataacaaaaactttattttcggagtgttctttgtataacttttccaagcttttacattaacgagcaggcctctgtcttaaaagggactcagtatattaatttctgcattttttaaatcaaatgaaaaacgtcaaattggaccaattgtcttggtttcttgattcatttatttgagaaaaaaacaatacaaagaaatgcattcatatcaaaattggaatagaaaggaaaacctatttttaagatatcaacctattttcacatcataaaacatctattacataaaataaaggtccaggcatagtggctcttgcctgtaatcccagcactttgggaggccgaggcaggtggatcactcaaggtcacaagtttgagaccagtctggccaacatggtgaaaccccatctctactaaaagtacaaatattagccgactatgtggcgggcacctgtaatcccacctactcgggaggctgaggcaggagaattgcttgaatccgggaagcagaggttgtagtgagccaagatcgtgccactgcactctatcctgggtaacagcgagactctgtcttaaaaataataacaataataataataataaagacttacttaaccaatattattgattaccagacttttatgaaagccaaagactgcttgctagtaggaaaaaatttcaaataataaccaaagctgaaaaatggtctgtcataaattatttccccggtaatttttgaaaggaaaaatgtataacaagtactatttacatatctgcatttaaaaaagcaattcttagaatacttcctttacattattctcctattttagacattttgtgaaagagaacaaattgtccagtggcctcctgtcagatcaacaattattatactccttaattccatgcaaatttaaatgaatgctataaaattttaaatctgtagcctgggtgtacgtttcactcaagttctcctactgaggactcttgactaacagcatactggcagtttcaccttaacctgctcgttaaataatgtgttggtgtgagatatcaggaatgtctcatgatatcacgtttaccatttacaccatctgcaaccatatgctattaataaaatggaaaaaaacaaaatggtcattttgcatatacttcactctgacctagtttagtccatgatactataattgtgagagcatatccagatgctgtgttctctatgtaaaacagtattgtccattcagaaatgtgtgacccttcatttatggatattgactatatgtaatgcagtgctatcccaatattttcaataaaggactttatgcattcagtgattttctttcccaaagattcattcatcaggtatttactgggtaccagagtgtttatttttgtgagaggatggtgaaatatccaagactaaacaaggtccagagctcatgttttcactgctgccctggaaactccctcttcattccctgtagccaccctcctaattgtctcatcagttcacggaaactgctctcattatggttaccagagacaactcttggctgtatctaccagtctcttctaatctttctagctgtgcaatgaatgacaacctcctcctcttaacatctgtcttaacctacttcataatttccgtaagggacactttactctctgataaattttctttggcatcctgacacctagcccctagatgttgggctagatgaagaccccaagcagtcttcattgcttcataattcctcagttcataagtccatatcaaaggacttgggtgggaggaggcaaccaaatgtttcttcagactctactgaaaatgattagatgcatccccgtgctacaagccaccagagacatcctgcactattataagtatgtcttcccttaatttgatctccctctccttgatgccttttaaagttttagagacacattgatatggtttggctgtgtgtccccaccaacatctcattggaattataatccccacatgttgagggagggacctagtgggatgtgattagaacatgggggcggtttcccccatgctgttctcatgatagtgagggagttttcatgagatctgttttaaaatgtttggcagttccaccctcaccctctcttctgctgccatgtaagatgtgccttgcttcccctttgccttctgccatgattgtaaatttcctgacgcctacccagccatgtgaaactgtgagtccattaaacctcttttctttataaataa

SEQ ID NO: 3 - DNA, 21 bp, Homo sapiens
  source 1..21: mol_type = other DNA
catgtacgttgctatccaggc

SEQ ID NO: 4 - DNA, 21 bp, Homo sapiens
  source 1..21: mol_type = other DNA
ctccttaatgtcacgcacgat

SEQ ID NO: 5 - DNA, 20 bp, Homo sapiens
  source 1..20: mol_type = other DNA
gccacacttctcactgctca

SEQ ID NO: 6 - DNA, 20 bp, Homo sapiens
  source 1..20: mol_type = other DNA
taggacaggcagcacctctt

SEQ ID NO: 7 - DNA, 19 bp, Homo sapiens
  source 1..19: mol_type = other DNA
cccgctgcaagatggaagg

SEQ ID NO: 8 - DNA, 20 bp, Homo sapiens
  source 1..20: mol_type = other DNA
cgagtagccatcctgcaact

SEQ ID NO: 9 - DNA, 20 bp, Mus musculus
  source 1..20: mol_type = other DNA
accttctacaatgagctgcg

SEQ ID NO: 10 - DNA, 20 bp, Mus musculus
  source 1..20: mol_type = other DNA
ctggatggctacgtacatgg

SEQ ID NO: 11 - DNA, 20 bp, Mus musculus
  source 1..20: mol_type = other DNA
gcaggcaaccagagaaagac

SEQ ID NO: 12 - DNA, 20 bp, Mus musculus
  source 1..20: mol_type = other DNA
cgttttgcctcctcaactgt

SEQ ID NO: 13 - DNA, 20 bp, Mus musculus
  source 1..20: mol_type = other DNA
atggaaagggtgtggttggg

SEQ ID NO: 14 - DNA, 20 bp, Mus musculus
  source 1..20: mol_type = other DNA
ctccaggcctgtacagagga

SEQ ID NO: 15 - DNA, 25 bp, Homo sapiens
  source 1..25: mol_type = other DNA
caccgttcaaatatatagcaggata

SEQ ID NO: 16 - DNA, 25 bp, Homo sapiens
  source 1..25: mol_type = other DNA
aaactatcctgctatatatttgaac

SEQ ID NO: 17 - DNA, 126 bp, Homo sapiens
  source 1..126: mol_type = other DNA
ctaaaccaagaaaatgatcatttgacagtgtttaccttggcaaggatactggccaggaggtgtcttcccattagggacatgactatggactcactatcctgctatatatttgaagacacagatcaa

SEQ ID NO: 18 - DNA, 20 bp, Homo sapiens
  source 1..20: mol_type = other DNA
cttcatggcctcctttggta

SEQ ID NO: 19 - DNA, 20 bp, Homo sapiens
  source 1..20: mol_type = other DNA
aaggatactggccaggaggt

SEQ ID NO: 20 - DNA, 20 bp, Homo sapiens
  source 1..20: mol_type = other DNA
ccaggttgaagtggaaagga

SEQ ID NO: 21 - DNA, 20 bp, Homo sapiens
  source 1..20: mol_type = other DNA
ggacaggcctcacctgatta

SEQ ID NO: 22 - DNA, 21 bp, Homo sapiens
  source 1..21: mol_type = other DNA
aaagctaggttcactggaggg

SEQ ID NO: 23 - DNA, 21 bp, Homo sapiens
  source 1..21: mol_type = other DNA
ggcatcgctgtgagactgtaa

SEQ ID NO: 24 - DNA, 20 bp, Homo sapiens
  source 1..20: mol_type = other DNA
tgggtggtatgcctagtgaa

SEQ ID NO: 25 - DNA, 20 bp, Homo sapiens
  source 1..20: mol_type = other DNA
ttaaggctgggctgtaaggt

SEQ ID NO: 26 - DNA, 20 bp, Homo sapiens
  source 1..20: mol_type = other DNA
acctcctggccagtatcctt

SEQ ID NO: 27 - DNA, 20 bp, Homo sapiens
  source 1..20: mol_type = other DNA
actcacatagtgcccctcct

SEQ ID NO: 28 - DNA, 20 bp, synthetic construct
  source 1..20: mol_type = other DNA
tttgggcaatgttacagcaa

SEQ ID NO: 29 - DNA, 20 bp, synthetic construct
  source 1..20: mol_type = other DNA
gaaatgccagggattcctta

SEQ ID NO: 30 - DNA, 109 bp, Homo sapiens
  source 1..109: mol_type = other DNA
cctctttatccagcttgatctgtgtcttcaaatatatagcaggatagggactccatagtcatgtccctgaatgggaagacacctcctggccagtatccttgccaaggta